(19) United States
(12) Patent Application Publication
(10) Pub. No.: US 2024/0336946 A1
(43) Pub. Date: Oct. 10, 2024

(54) SOLID-PHASE GLYCAN REMODELING OF GLYCOPROTEINS

(71) Applicant: MERCK SHARP & DOHME LLC, RAHWAY, NJ (US)

(72) Inventors: YEN-PANG HSU, Cambridge, MA (US); BENJAMIN F. MANN, Westfield, NJ (US); SHUWEN SUN, Clark, NJ (US); DEEPTAK VERMA, Feasterville-Trevose, PA (US)

(73) Assignee: MERCK SHARP & DOHME LLC, RAHWAY, NJ (US)

(21) Appl. No.: 18/554,242

(22) PCT Filed: Apr. 11, 2022
(86) PCT No.: PCT/US2022/024194

§ 371 Date: Oct. 6, 2023

Related U.S. Application Data
(60) Provisional application No. 63/175,602, filed on Apr. 16, 2021.

(51) Int. Cl. C12P 21/00

(52) U.S. Cl. CPC C12P 21/005

(57) ABSTRACT

A solid-phase glycan remodeling (SPGR) system for the glycoengineering of glycoproteins to provide glycoprotein compositions comprising particular predominant glycoforms is described.

### BACKGROUND OF THE INVENTION

### (1) Field of the Invention

The present invention relates to a solid-phase glycan remodeling (SPGR) system for the glycoengineering of glycoproteins to provide glycoprotein compositions comprising particular predominant glycoforms.

### (2) Description of Related Art

Protein glycosylation is a key determinant of the physical and biochemical properties of proteins in eukaryotic systems (Varki et al. in Essentials of Glycobiology, Cold Spring Harbor Laboratory Press, The Consortium of Glycobiology Editors, La Jolla, C A 2015); Spiro, Glycobiology 12, 43R-56R, doi:10.1093/glycob/12.4.43R (2002); Varki, A. in Glycobiology Vol. 27 3-49 (2017)). According to glycoproteomic analyses, over 1% of the human genome encodes glycosylation-related enzymes and more than 50% of human proteins are glycosylated (Apweiler et al., Biochim. Biophys. Acta 1473, 4-8, doi:10.1016/s0304-4165(99)00165-8 (1999)). Glycoproteins carry structurally diverse oligosaccharides, called glycans, that are involved at the interface of protein-biomolecular interactions and thus determine protein stability, selectivity, and activity. The significance of protein glycosylation to biological systems has been exemplified by several diseases associated with various cancers and the immune system (Reily et al., Nature Reviews Nephrology 15, 346-366, doi:10.1038/s41581-019-0129-4 (2019); Maverakis et al., J. Autoimmun. 57, 1-13, doi:10.1016/j.jaut.2014.12.002 (2015); Reiding et al., Front Med (Lausanne) 4, doi:10.3389/fmed.2017.00241 (2017); van de Bovenkamp et al., Journal of Immunology 196, 1435-1441, doi:10.4049/jimmunol.1502136 (2016); Munkley, Oncol. Lett. 17, 2569-2575, doi:10.3892/ol.2019.9885 (2019); Pinho & Reis, Nat. Rev. Cancer 15, 540-555, doi:10.1038/nrc3982 (2015); Schultz et al. Cancer Res. 76, 3978-3988, doi:10.1158/0008-5472.can-15-2834 (2016); Nairn et al., J. Biol. Chem. 287, 37835-37856, doi:10.1074/jbc.M112.405233 (2012)). For example, patients with rheumatoid arthritis were found to have an increased galactosylation level in their serum immunoglobulin G (IgG), though the mechanism remains elusive (Reiding et al., Front Med (Lausanne) 4, doi:10.3389/fmed.2017.00241 (2017)). Unsurprisingly, it follows that insights into the structure and function of glycans have yielded a profound impact on the development of therapeutic glycoproteins (Mimura et al., Protein Cell 9, 47-62, doi:10.1007/s13238-017-0433-3 (2018)). Manipulating glycan structures presents an effective strategy to improve their efficacy and safety by modulating immunological responses, circulatory half-life, and effector functions (Goetze et al., Glycobiology 21, 949-959, doi:10.1093/glycob/cwr027 (2011); Higel et al., Eur. J. Pharma. Biopharma. 100, 94-100, doi:https://doi.org/10.1016/j.ejpb.2016.01.005 (2016); Chiang et al., Curr. Opin. Struct. Biol. 40, 104-111, doi:10.1016/j.sbi.2016.08.008 (2016)). Thus, glycoengineering represents a versatile tool and a great opportunity to create better medicines through glycan remodeling. To achieve this goal, technologies that confer control of protein glycosylation profiles are essential.

To date, however, tools to access the diverse array of glycan structures displayed in nature remain scarce, and methods that produce a high yield of the desired glycoforms have proven to be a still greater challenge to develop despite decades of study (Wong, J. Organic Chemistry 70, 4219-4225, doi:10.1021/jo050278f (2005); Zhang et al. Drug Discovery Today 21, 740-765, doi:https://doi.org/10.1016/j.drudis.2016.01.006 (2016)). Through traditional synthetic approaches, several common glycoforms have been accessed (Fairbanks, Beilstein J. Organic Chemistry 14, 416-429, doi:10.3762/bjoc.14.30 (2018); Nagasaki et al., J. Organic Chemistry 81, 10600-10616, doi:10.1021/acs.joc.6b02106 (2016); Tang et al., J. Organic Chemistry 80, 10482-10489, doi:10.1021/acs.joc.5b01428 (2015)). These structurally-defined glycans can be installed onto glycoproteins through endoglycosidase and glycosynthase activities (Heidecke et al., Chembiochem 9, 2045-2051, doi:10.1002/cbic.200800214 (2008); Umekawa et al., J. Biol. Chem. 285, 511-521, doi:10.1074/jbc.M109.059832 (2010); Lin et al., Proc Natl Acad Sci USA 112, 10611-10616, doi:10.1073/pnas.1513456112 (2015)). While this approach has advanced our ability to control protein glycosylation, the preparation of synthetic glycans becomes increasingly difficult as the number of saccharide units increases. As a result, the installation of synthetic glycans is not practical for many applications. On the other hand, genetic engineering has been applied for controlled glycan biosynthesis by either knocking out or introducing certain glycosylation enzymes in the host cells (Malphettes et al., Biotechnol. Bioeng. 106, 774-783, doi:10.1002/bit.22751 (2010); Yang et al. Nat. Biotechnol. 33, 842-844, doi:10.1038/nbt.3280 (2015)). This strategy enables in vivo glycan remodeling and has been demonstrated to produce humanized glycoproteins in non-human cell lines (Liu et al., Proc Natl Acad Sci USA 115, 720-725, doi:10.1073/pnas.1718172115 (2018)). However, the optimization of this strategy has been impeded by the complexity of engineering glycosylation pathways. Also, micro-heterogeneity is often generated during in vivo glycan formation, which, although it is comparable to the natural phenomenon, does not provide exquisite control over the molecular structure (Higel et al., Eur. J. Pharma. Biopharma. 100, 94-100, doi:https://doi.org/10.1016/j.ejpb.2016.01.005 (2016); Kolarich et al., Nature Protocols 7, 1285-1298, doi:10.1038/nprot.2012.062 (2012)).

In recent decades, our understanding of the in vitro activity of glycosylation enzymes is growing rapidly (Peng et al., Chembiochem: a European journal of chemical biology 18, 2306-2311, doi:10.1002/cbic.201700292 (2017); Lairson et al., Annual Rev. Biochemistry 77, 521-555, doi:10.1146/annurev.biochem.76.061005.092322 (2008); Sjögren et al., Glycobiology 30, 254-267, doi:10.1093/glycob/cwz085 (2019)). Some of the enzymes can even function on intact glycoproteins, which opens a new window for glycan remodeling (Hamilton et al., Scientific Reports 7, 15907, doi:10.1038/s41598-017-15891-8 (2017); Huang et al., J. Am. Chem. Soc. 134, 12308-12318, doi:10.1021/ja3051266 (2012); Rich & Withers, Nat. Chem. Biol. 5, 206-215, doi:10.1038/nchembio.148 (2009); Ochiai et al., J. Am. Chem. Soc. 130, 13790-13803, doi:10.1021/ja805044x (2008); McArthur et al., Biochem.Society Transactions 44, 129-142, doi:10.1042/bst20150200 (2016); Tang et al., Organic & Biomolec. Chem. 14, 9501-9518, doi:10.1039/C60B01751G (2016); Li, T. et al. Nature Chemistry 11, 229-236, doi:10.1038/s41557-019-0219-8 (2019); Barb, & Prestegard, Nat Chem Biol 7, 147-153, doi:10.1038/nchembio.511 (2011)).

To further leverage the use of these enzymes, three primary challenges need to be addressed. First, most of the enzymes applied to glycan engineering have been studied based on the use of synthetic oligosaccharides and/or denatured glycoproteins as substrates. Whether or not a suite of enzymes can remodel glycosylation on intact glycoproteins is a question that persists. Second, preserving the integrity and functions of the substrates after the enzymatic reactions is advantageous, especially for therapeutic glycoproteins. Protocols with high biocompatibility are thus required. Third, to construct complex glycan structures, successive reactions using different enzymes are needed. These enzymes might require very different working conditions, such as pH and temperature. Therefore, one would need to repeat the buffer swapping and product purification processes between the enzymatic reactions, which is highly labor-intensive and time-consuming. Together, to address these issues, novel platforms that enable efficient, successive enzymatic glycan remodeling with high biocompatibility to the substrates are in great demand.

### BRIEF SUMMARY OF THE INVENTION

The present invention provides a solid-phase glycan remodeling (SPGR) system wherein glycosylation enzymatic reactions are carried out on glycoprotein substrates immobilized on resins. The SPGR system of the present invention enables efficient reaction swapping, substrate purification, and the recovery of both products and engineering enzymes. The present invention is particularly useful for glycoengineering immunoglobulins (e.g., antibodies or IgG) to provide antibodies that have predominantly particular glycoform profiles. The SPGR system of the present invention enables harmonization of antibody glycans into one or more of ten different glycoforms, including non-canonical structures, in 48 hours with an average conversion ratio greater than 95%. Physical and biochemical analyses indicate that the SPGR-engineered antibodies preserved integrity and functionality, suggesting that the SPGR system of the present invention has high biocompatibility to the substrates.

The present invention provides a method for remodeling the N-glycans of a composition of N-glycosylated glycoproteins, the steps comprising: (a) providing an aqueous composition of N-glycosylated glycoproteins; (b) immobilizing the N-glycosylated glycoproteins on a solid support; (c) reacting the immobilized N-glycosylated glycoproteins with a glycosylation enzyme or sequentially with two or more glycosylation enzymes to produce immobilized N-glycosylated glycoproteins comprising remodeled N-glycans; (d) eluting the N-glycosylated glycoproteins comprising the remodeled N-glycans from the solid support to provide a composition of the N-glycosylated glycoproteins comprising remodeled N-glycans.

In a further embodiment, the solid support comprises irreversibly affixed thereon a multiplicity of capture moieties that specifically bind the N-glycosylated glycoprotein.

In a further embodiment, the N-glycosylated glycoprotein comprises an antibody or antigen-binding fragment.

In a further embodiment, the multiplicity of capture moieties comprises protein A and the N-glycosylated glycoprotein comprises an antibody.

In a further embodiment, the one or more glycosylation enzymes are selected from the group consisting of exoglycosidases, endoglycosidases, and glycosyltransferases.

In a further embodiment, the exoglycosidases are selected from the group consisting of neuraminidases, galactosidases, fucosidases, and N-acetyl-glucosaminidases; and, the glycosyltransferases are selected from the group consisting of N-acetylglucosaminidyltransferases (GnTs), galactosyltransferases (GalTs), fucosyltransferases (FuTs), and sialyltransferases (SiaTs).

In a further embodiment, the endoglycosidases are selected from the group consisting of endoglycosylase A, D, F2, F3, M, and S.

In a further embodiment, the SiaTs are selected from the group consisting of a2-6 SiaT and α2-3 SiaT: the GalTs comprise β1-4GalT: the FuTs comprise α1-6 FuT; and, the GnTs are selected from the group consisting of GnTI, GnTII, GnTIII, GnTIV, and GnTV.

In a further embodiment, the immobilized N-glycosylated glycoproteins are reacted with an exoglycosidase or sequentially with two or more exoglycosidases to produce first immobilized N-glycosylated glycoproteins wherein the remodeled N-glycans thereof comprise two terminal mannose residues at the non-reducing end of the N-glycan, one terminal mannose linked to a central mannose residue in an α1-3 linkage and the other terminal mannose linked to the same central mannose in an α1-6 linkage, and the central mannose is linked to a chitobiose core in a 1-4 linkage wherein the GlcNAc residue at the reducing end of the chitobiose core is linked to an asparagine residue of an N-glycosylation site in the N-glycosylated glycoprotein.

In a further embodiment, the first immobilized N-glycosylated glycoproteins are reacted with a GnTI in the presence of UDP-GlcNAc to produce second immobilized N-glycosylated glycoproteins wherein the remodeled N-glycans thereof further comprise a GlcNAc residue linked in a β1-2 linkage to the non-reducing end of the α1-3-linked mannose residue.

In a further embodiment, the second immobilized N-glycosylated glycoproteins are reacted with a GalT in the presence of UDP-galactose to produce third immobilized N-glycosylated glycoproteins wherein the remodeled N-glycans thereof further comprise a galactose residue linked in a β1-4 linkage to the non-reducing end of the GlcNAc residue linked to the non-reducing end of the α1-3-linked mannose residue.

In a further embodiment, the third immobilized N-glycosylated glycoproteins are reacted with a SiaT in the presence of CMP-sialic acid to produce fourth immobilized N-glycosylated glycoproteins wherein the remodeled N-glycans thereof further comprise a sialic acid residue linked to the non-reducing end of the galactose residue linked to the non-reducing end of the GlcNAc residue linked in an α2-3 or α2-6 linkage to the non-reducing end of the α1-3-linked mannose residue.

In a further embodiment, the second immobilized N-glycosylated glycoproteins are reacted with a GnTII in the presence of UDP-GlcNAc to produce third immobilized N-glycosylated glycoproteins wherein the remodeled N-glycans thereof further comprise a GlcNAc residue linked in a β1-2 linkage to the non-reducing end of the α1-6-linked mannose residue.

In a further embodiment, the third immobilized N-glycosylated glycoproteins are reacted with a GalT in the presence of UDP-galactose to produce fourth immobilized N-glycosylated glycoproteins wherein the remodeled N-glycans thereof further comprise (a) a galactose residue linked in a β1-4 linkage to the non-reducing end of the GlcNAc residue linked in a β1-2 linkage to the non-reducing end of the α1-3-linked mannose residue, (b) a galactose residue linked in a β1-4 linkage to the non-reducing end of the GlcNAc residue linked in a β1-2 linkage to the non-reducing end of the α1-6-linked mannose residue, or (c) a galactose residue linked in a β1-4 linkage to the non-reducing end of the GlcNAc residue linked in a β1-2 linkage to the non-reducing end of the α1-3-linked mannose residue and a galactose residue linked in a β1-4 linkage to the non-reducing end of the GlcNAc residue linked in a β1-2 linkage to the non-reducing end of the α1-6-linked mannose residue.

In a further embodiment, the fourth immobilized N-glycosylated glycoproteins are reacted with a SiaT in the presence of CMP-sialic acid to produce fifth immobilized N-glycosylated glycoproteins wherein the remodeled N-glycans thereof further comprise (i) a sialic acid residue linked in an α2-3 or α2-6 linkage to the non-reducing end of the galactose residue linked in a β1-4 linkage to the non-reducing end of the GlcNAc residue linked in a β1-2 linkage to the non-reducing end of the α1-3-linked mannose residue, (ii) a sialic acid residue linked in an α2-3 or α2-6 linkage to the non-reducing end of the galactose residue linked in a β1-4 linkage to the non-reducing end of the GlcNAc residue linked in a β1-2 linkage to the non-reducing end of the α1-6-linked mannose residue, or (iii) a sialic acid residue linked in an α2-3 or α2-6 linkage to the non-reducing end of the galactose residue linked in a 1-4 linkage to the non-reducing end of the GlcNAc residue linked in a β1-2 linkage to the non-reducing end of the α1-3-linked mannose residue and a sialic acid residue linked in an α2-3 or α2-6 linkage to the non-reducing end of the galactose residue linked in a β1-4 linkage to the non-reducing end of the GlcNAc residue linked in a β1-2 linkage to the non-reducing end of the α1-6-linked mannose residue.

In a further embodiment, the third immobilized N-glycosylated glycoproteins are reacted with a GnTIII in the presence of UDP-GlcNAc to produce fourth immobilized N-glycosylated glycoproteins wherein the remodeled N-glycans thereof further comprise a GlcNAc residue linked in a 1-4 linkage to the non-reducing end of the central mannose residue.

In a further embodiment, the fourth immobilized N-glycosylated glycoproteins are reacted with a GalT in the presence of UDP-galactose to produce fifth immobilized N-glycosylated glycoproteins wherein the remodeled N-glycans thereof further comprise (i) a galactose residue linked in a β1-4 linkage to the non-reducing end of the GlcNAc residue linked in a β1-2 linkage to the non-reducing end of the α1-3-linked mannose residue, (ii) a galactose residue linked in a β1-4 linkage to the non-reducing end of the GlcNAc residue linked in a β1-2 linkage to the non-reducing end of the α1-6-linked mannose residue, or (iii) a galactose residue linked in a β1-4 linkage to the non-reducing end of the GlcNAc residue linked to the non-reducing end of the α1-3-linked mannose residue and a galactose residue linked in a β1-4 linkage to the non-reducing end of the GlcNAc residue linked in a 1-2 linkage to the non-reducing end of the α1-6-linked mannose residue.

In a further embodiment, the fifth immobilized N-glycosylated glycoproteins are reacted with a SiaT in the presence of CMP-sialic acid to produce sixth immobilized N-glycosylated glycoproteins wherein the remodeled N-glycans thereof further comprise (i) a sialic acid residue linked in an α2-3 or α2-6 linkage to the non-reducing end of the galactose residue linked in a β1-4 linkage to the non-reducing end of the GlcNAc residue linked in a β1-2 linkage to the non-reducing end of the α1-3-linked mannose residue, (ii) a sialic acid residue linked in an α2-3 or α2-6 linkage to the non-reducing end of the galactose residue linked in a β1-4 linkage to the non-reducing end of the GlcNAc residue linked in a β1-2 linkage to the non-reducing end of the α1-6-linked mannose residue, or (iii) a sialic acid residue linked in an α2-3 or α2-6 linkage to the non-reducing end of the galactose residue linked in a β1-2 linkage to the non-reducing end of the GlcNAc residue linked in a β1-2 linkage to the non-reducing end of the α1-3-linked mannose residue and a sialic acid residue linked in an α2-3 or α2-6 linkage to the non-reducing end of the galactose residue linked in a β1-2 linkage to the non-reducing end of the GlcNAc residue linked in a β1-2 linkage to the non-reducing end of the α1-6-linked mannose residue.

In a further embodiment, the third immobilized N-glycosylated glycoproteins are reacted with a GnTIV in the presence of UDP-GlcNAc to produce fourth immobilized N-glycosylated glycoproteins wherein the remodeled N-glycans thereof further comprise a GlcNAc residue linked in a β1-4 linkage to the non-reducing end of the α1-3-linked mannose residue.

In a further embodiment, the fourth immobilized N-glycosylated glycoproteins are reacted with a GalT in the presence of UDP-galactose to produce fifth immobilized N-glycosylated glycoproteins wherein the remodeled N-glycans thereof further comprise (i) a galactose residue linked in a β1-4 linkage to the non-reducing end of the GlcNAc residue linked in a β1-2 linkage to the non-reducing end of the α1-3-linked mannose residue;

(ii) a galactose residue linked in a β1-4 linkage to the non-reducing end of the GlcNAc residue linked in a β1-2 linkage to the non-reducing end of the α1-6-linked mannose residue; (iii) a galactose residue linked in a β1-4 linkage to the non-reducing end of the GlcNAc residue linked in a β1-4 linkage to the non-reducing end of the α1-3-linked mannose residue; or (iv) a combination of two or more selected from the group consisting of (i), (ii), and (iii).

In a further embodiment, the fifth immobilized N-glycosylated glycoproteins are reacted with a SiaT in the presence of CMP-sialic acid to produce sixth immobilized N-glycosylated glycoproteins wherein the remodeled N-glycans thereof further comprise (i) a sialic acid residue linked in an α2-3 or α2-6 linkage to the non-reducing end of the galactose residue linked in a β1-4 linkage to the non-reducing end of the GlcNAc residue linked in a β1-2 linkage to the non-reducing end of the α1-3-linked mannose residue; (ii) a sialic acid residue linked in an α2-3 or α2-6 linkage to the non-reducing end of the galactose residue linked in a β1-4 linkage to the non-reducing end of the GlcNAc residue linked in a β1-2 linkage to the non-reducing end of the α1-6-linked mannose residue; (iii) a sialic acid residue linked in an α2-3 or α2-6 linkage to the non-reducing end of the galactose residue linked in a β1-4 linkage to the non-reducing end of the GlcNAc residue linked in a β1-4 linkage to the non-reducing end of the α1-3-linked mannose residue; or (iv) a combination of two or more selected from the group consisting of (i), (ii), and (iii).

In a further embodiment, the fourth immobilized N-glycosylated glycoproteins are reacted with a GnTV in the presence of UDP-GlcNAc to produce fifth immobilized N-glycosylated glycoproteins wherein the remodeled N-glycans thereof further comprise a GlcNAc residue linked in a β1-6 linkage to the non-reducing end of the α1-6-linked mannose residue.

In a further embodiment, the fifth immobilized N-glycosylated glycoproteins are reacted with a GalT in the presence of UDP-galactose to produce sixth immobilized N-glycosylated glycoproteins wherein the remodeled N-glycans thereof further comprise (i) a galactose residue linked in a β1-4 linkage to the non-reducing end of the GlcNAc residue linked in a β1-2 linkage to the non-reducing end of the α1-3-linked mannose residue;

(ii) a galactose residue linked in a β1-4 linkage to the non-reducing end of the GlcNAc residue linked in a β1-2 linkage to the non-reducing end of the α1-6-linked mannose residue; (iii) a galactose residue linked in a β1-4 linkage to the non-reducing end of the GlcNAc residue linked in a β1-4 linkage to the non-reducing end of the α1-3-linked mannose residue; (iv) a galactose residue linked in a β1-4 linkage to the non-reducing end of the GlcNAc residue linked in a β1-6 linkage to the non-reducing end of the α1-6-linked mannose residue; or (v) a combination of two or more selected from the group consisting of (i), (ii), (iii), and (iv).

In a further embodiment, the sixth immobilized N-glycosylated glycoproteins are reacted with a SiaT in the presence of CMP-sialic acid to produce seventh immobilized N-glycosylated glycoproteins wherein the remodeled N-glycans thereof further comprise

(i) a sialic acid residue linked in an α2-3 or α2-6 linkage to the non-reducing end of the galactose residue linked in a β1-4 linkage to the non-reducing end of the GlcNAc residue linked in a β1-2 linkage to the non-reducing end of the α1-3-linked mannose residue; (ii) a sialic acid residue linked in an α2-3 or α2-6 linkage to the non-reducing end of the galactose residue linked in a β1-4 linkage to the non-reducing end of the GlcNAc residue linked in a β1-2 linkage to the non-reducing end of the α1-6-linked mannose residue; (iii) a sialic acid residue linked in an α2-3 or α2-6 linkage to the non-reducing end of the galactose residue linked in a β1-4 linkage to the non-reducing end of the GlcNAc residue linked in a β1-4 linkage to the non-reducing end of the α1-3-linked mannose residue; (iv) a sialic acid residue linked in an α2-3 or α2-6 linkage to the non-reducing end of the galactose residue linked in a 1-4 linkage to the non-reducing end of the GlcNAc residue linked in a β1-6 linkage to the non-reducing end of the α1-6-linked mannose residue; or (v) a combination of two or more selected from the group consisting of (i), (ii), (iii), and (iv).

The present invention further provides a method for remodeling the N-glycans of a composition of N-glycosylated glycoproteins, the steps comprising: (a) providing an aqueous composition of N-glycosylated glycoproteins and two or more solid supports, each solid support having immobilized thereon a multiplicity of glycosylation enzymes and wherein no solid support has immobilized thereon the same multiplicity of glycosylation enzymes; and (b) applying the composition of N-glycosylated glycoproteins sequentially to each solid support for a time sufficient to remodel the N-glycans of the glycoproteins to produce the composition of N-glycosylated glycoproteins comprising remodeled N-glycans. A further step may include separating the composition and purifying the composition.

In a further embodiment, the multiplicity of glycosylation enzymes are irreversibly affixed to the solid support or bound to capture moieties that specifically bind the glycosylation enzymes.

In a further embodiment, the N-glycosylated glycoprotein comprises an antibody or antigen binding fragment.

In a further embodiment, the one or more glycosylation enzymes are selected from the group consisting of exoglycosidases, endoglycosidases, and glycosyltransferases.

In a further embodiment, the exoglycosidases are selected from the group consisting of neuraminidases, galactosidases, fucosidases, and N-acetyl-glucosaminidases; and, the glycosyltransferases are selected from the group consisting of N-acetylglucosaminidyltransferases (GnTs), galactosyltransferases (GalTs), fucosyltransferases (FuTs), and sialyltransferases (SiaTs).

In a further embodiment, the SiaTs are selected from the group consisting of α2-6 SiaT and α2-3 SiaT: the GalTs comprise β1-4GalT: the FuTs comprise α1-6 FuT; and, the GnTs are selected from the group consisting of GnTI, GnTII, GnTIII, GnTIV, and GnTV.

The present invention may use any combination of solid supports comprising a multiplicity of N-glycans immobilized thereon, the combination taking into account the particular characteristics of the glycosylation enzymes.

In a further embodiment, the composition of N-glycosylated glycoproteins are sequentially applied to a solid support comprising a multiplicity of neuraminidases, a solid support comprising a multiplicity of galactosidases, and a solid support comprising a multiplicity of N-acetyl-glucosaminidases to produce a first composition of N-glycosylated glycoproteins wherein the remodeled N-glycans thereof comprise two terminal mannose residues at the non-reducing end of the N-glycan, one terminal mannose linked to a central mannose residue in an α1-3 linkage and the other terminal mannose linked to the same central mannose in an α1-6 linkage, and the central mannose is linked to a chitobiose core in a β1-4 linkage wherein the GlcNAc residue at the reducing end of the chitobiose core is linked to an asparagine residue of an N-glycosylation site in the N-glycosylated glycoprotein.

In a further embodiment, the first composition of N-glycosylated glycoproteins are applied to a solid support comprising a multiplicity of GnTIs in the presence of UDP-GlcNAc to produce a second composition of N-glycosylated glycoproteins wherein the remodeled N-glycans thereof further comprise a GlcNAc residue linked in a β1-2 linkage to the non-reducing end of the α1-3-linked mannose residue.

In a further embodiment, the second composition of N-glycosylated glycoproteins are applied to a solid support comprising a multiplicity of GalTs in the presence of UDP-galactose to produce a third composition of N-glycosylated glycoproteins wherein the remodeled N-glycans thereof further comprise a galactose residue linked in a β1-4 linkage to the non-reducing end of the GlcNAc residue linked to the non-reducing end of the α1-3-linked mannose residue.

In a further embodiment, the third composition of N-glycosylated glycoproteins are applied to a solid support comprising a multiplicity of SiaT in the presence of CMP-sialic acid to produce a fourth composition of N-glycosylated glycoproteins wherein the remodeled N-glycans thereof further comprise a sialic acid residue linked to the non-reducing end of the galactose residue linked to the non-reducing end of the GlcNAc residue linked in an α2-3 or α2-6 linkage to the non-reducing end of the α1-3-linked mannose residue.

In a further embodiment, the second composition of N-glycosylated glycoproteins are applied to a solid support comprising a multiplicity of GnTIIs in the presence of UDP-GlcNAc to produce a third composition of N-glycosylated glycoproteins wherein the remodeled N-glycans thereof further comprise a GlcNAc residue linked in a β1-2 linkage to the non-reducing end of the α1-6-linked mannose residue.

In a further embodiment, the third composition of N-glycosylated glycoproteins are applied to a solid support comprising a multiplicity of GalT in the presence of UDP-galactose to produce a fourth composition of N-glycosylated glycoproteins wherein the remodeled N-glycans thereof further comprise (a) a galactose residue linked in a β1-4 linkage to the non-reducing end of the GlcNAc residue linked in a 1-2 linkage to the non-reducing end of the α1-3-linked mannose residue, (b) a galactose residue linked in a β1-4 linkage to the non-reducing end of the GlcNAc residue linked in a β1-2 linkage to the non-reducing end of the α1-6-linked mannose residue, or (c) a galactose residue linked in a β1-4 linkage to the non-reducing end of the GlcNAc residue linked in a β1-2 linkage to the non-reducing end of the α1-3-linked mannose residue and a galactose residue linked in a β1-4 linkage to the non-reducing end of the GlcNAc residue linked in a β1-2 linkage to the non-reducing end of the α1-6-linked mannose residue.

In a further embodiment, the fourth composition of N-glycosylated glycoproteins are applied to a solid support comprising a multiplicity of SiaTs in the presence of CMP-sialic acid to produce a fifth composition of N-glycosylated glycoproteins wherein the remodeled N-glycans thereof further comprise (i) a sialic acid residue linked in an α2-3 or α2-6 linkage to the non-reducing end of the galactose residue linked in a β1-4 linkage to the non-reducing end of the GlcNAc residue linked in a β1-2 linkage to the non-reducing end of the α1-3-linked mannose residue, (ii) a sialic acid residue linked in an α2-3 or α2-6 linkage to the non-reducing end of the galactose residue linked in a β1-4 linkage to the non-reducing end of the GlcNAc residue linked in a β1-2 linkage to the non-reducing end of the α1-6-linked mannose residue, or (iii) a sialic acid residue linked in an α2-3 or α2-6 linkage to the non-reducing end of the galactose residue linked in a β1-4 linkage to the non-reducing end of the GlcNAc residue linked in a β1-2 linkage to the non-reducing end of the α1-3-linked mannose residue and a sialic acid residue linked in an α2-3 or α2-6 linkage to the non-reducing end of the galactose residue linked in a β1-4 linkage to the non-reducing end of the GlcNAc residue linked in a β1-2 linkage to the non-reducing end of the α1-6-linked mannose residue.

In a further embodiment, the third composition of N-glycosylated glycoproteins are applied to a solid support comprising a multiplicity of GnTIIIs in the presence of UDP-GlcNAc to produce a fourth composition of N-glycosylated glycoproteins wherein the remodeled N-glycans thereof further comprise a GlcNAc residue linked in a β1-4 linkage to the non-reducing end of the central mannose residue.

In a further embodiment, the fourth composition of N-glycosylated glycoproteins are applied to a solid support comprising a multiplicity of GalTs in the presence of UDP-galactose to produce a fifth composition of N-glycosylated glycoproteins wherein the remodeled N-glycans thereof further comprise (i) a galactose residue linked in a β1-4 linkage to the non-reducing end of the GlcNAc residue linked in a β1-2 linkage to the non-reducing end of the α1-3-linked mannose residue, (ii) a galactose residue linked in a β1-4 linkage to the non-reducing end of the GlcNAc residue linked in a 1-2 linkage to the non-reducing end of the α1-6-linked mannose residue, or (iii) a galactose residue linked in a β1-4 linkage to the non-reducing end of the GlcNAc residue linked to the non-reducing end of the α1-3-linked mannose residue and a galactose residue linked in a β1-4 linkage to the non-reducing end of the GlcNAc residue linked in a β1-2 linkage to the non-reducing end of the α1-6-linked mannose residue.

In a further embodiment, the fifth composition of N-glycosylated glycoproteins are applied to a solid support comprising a multiplicity of SiaTs in the presence of CMP-sialic acid to produce a sixth composition of N-glycosylated glycoproteins wherein the remodeled N-glycans thereof further comprise (i) a sialic acid residue linked in an α2-3 or α2-6 linkage to the non-reducing end of the galactose residue linked in a β1-4 linkage to the non-reducing end of the GlcNAc residue linked in a β1-2 linkage to the non-reducing end of the α1-3-linked mannose residue, (ii) a sialic acid residue linked in an α2-3 or α2-6 linkage to the non-reducing end of the galactose residue linked in a β1-4 linkage to the non-reducing end of the GlcNAc residue linked in a β1-2 linkage to the non-reducing end of the α1-6-linked mannose residue, or (iii) a sialic acid residue linked in an α2-3 or α2-6 linkage to the non-reducing end of the galactose residue linked in a β1-2 linkage to the non-reducing end of the GlcNAc residue linked in a β1-2 linkage to the non-reducing end of the α1-3-linked mannose residue and a sialic acid residue linked in an α2-3 or α2-6 linkage to the non-reducing end of the galactose residue linked in a β1-2 linkage to the non-reducing end of the GlcNAc residue linked in a β1-2 linkage to the non-reducing end of the α1-6-linked mannose residue.

In a further embodiment, the third composition of N-glycosylated glycoproteins are applied to a solid support comprising a multiplicity of GnTIVs in the presence of UDP-GlcNAc to produce a fourth composition of N-glycosylated glycoproteins wherein the remodeled N-glycans thereof further comprise a GlcNAc residue linked in a β1-4 linkage to the non-reducing end of the α1-3-linked mannose residue.

In a further embodiment, the fourth composition of N-glycosylated glycoproteins are applied to a solid support comprising a multiplicity of GalTs in the presence of UDP-galactose to produce a fifth composition of N-glycosylated glycoproteins wherein the remodeled N-glycans thereof further comprise (i) a galactose residue linked in a β1-4 linkage to the non-reducing end of the GlcNAc residue linked in a 1-2 linkage to the non-reducing end of the α1-3-linked mannose residue; (ii) a galactose residue linked in a β1-4 linkage to the non-reducing end of the GlcNAc residue linked in a β1-2 linkage to the non-reducing end of the α1-6-linked mannose residue; (iii) a galactose residue linked in a β1-4 linkage to the non-reducing end of the GlcNAc residue linked in a β1-4 linkage to the non-reducing end of the α1-3-linked mannose residue; or (iv) a combination of two or more selected from the group consisting of (i), (ii), and (iii).

In a further embodiment, the fifth composition of N-glycosylated glycoproteins are applied to a solid support comprising a multiplicity of SiaTs in the presence of CMP-sialic acid to produce a sixth composition of N-glycosylated glycoproteins wherein the remodeled N-glycans thereof further comprise (i) a sialic acid residue linked in an α2-3 or α2-6 linkage to the non-reducing end of the galactose residue linked in a β1-4 linkage to the non-reducing end of the GlcNAc residue linked in a 1-2 linkage to the non-reducing end of the α1-3-linked mannose residue; (ii) a sialic acid residue linked in an α2-3 or α2-6 linkage to the non-reducing end of the galactose residue linked in a β1-4 linkage to the non-reducing end of the GlcNAc residue linked in a β1-2 linkage to the non-reducing end of the α1-6-linked mannose residue; (iii) a sialic acid residue linked in an α2-3 or α2-6 linkage to the non-reducing end of the galactose residue linked in a β1-4 linkage to the non-reducing end of the GlcNAc residue linked in a β1-4 linkage to the non-reducing end of the α1-3-linked mannose residue; or (iv) a combination of two or more selected from the group consisting of (i), (ii), and (iii).

In a further embodiment, the fourth composition of N-glycosylated glycoproteins are applied to a solid support comprising a multiplicity of GnTVs in the presence of UDP-GlcNAc to produce a fifth composition of N-glycosylated glycoproteins wherein the remodeled N-glycans thereof further comprise a GlcNAc residue linked in a β1-6 linkage to the non-reducing end of the α1-6-linked mannose residue.

In a further embodiment, the fifth composition of N-glycosylated glycoproteins are applied to a solid support comprising a multiplicity of GalTs in the presence of UDP-galactose to produce a sixth composition of N-glycosylated glycoproteins wherein the remodeled N-glycans thereof further comprise (i) a galactose residue linked in a β1-4 linkage to the non-reducing end of the GlcNAc residue linked in a β1-2 linkage to the non-reducing end of the α1-3-linked mannose residue; (ii) a galactose residue linked in a β1-4 linkage to the non-reducing end of the GlcNAc residue linked in a β1-2 linkage to the non-reducing end of the α1-6-linked mannose residue; (iii) a galactose residue linked in a β1-4 linkage to the non-reducing end of the GlcNAc residue linked in a β1-4 linkage to the non-reducing end of the α1-3-linked mannose residue; (iv) a galactose residue linked in a β1-4 linkage to the non-reducing end of the GlcNAc residue linked in a β1-6 linkage to the non-reducing end of the α1-6-linked mannose residue; or (v) a combination of two or more selected from the group consisting of (i), (ii), (iii), and (iv).

In a further embodiment, the sixth composition of N-glycosylated glycoproteins are applied to a solid support comprising a multiplicity of SiaTs in the presence of CMP-sialic acid to produce a seventh composition of N-glycosylated glycoproteins wherein the remodeled N-glycans thereof further comprise (i) a sialic acid residue linked in an α2-3 or α2-6 linkage to the non-reducing end of the galactose residue linked in a β1-4 linkage to the non-reducing end of the GlcNAc residue linked in a β1-2 linkage to the non-reducing end of the α1-3-linked mannose residue; (ii) a sialic acid residue linked in an α2-3 or α2-6 linkage to the non-reducing end of the galactose residue linked in a β1-4 linkage to the non-reducing end of the GlcNAc residue linked in a β1-2 linkage to the non-reducing end of the α1-6-linked mannose residue; (iii) a sialic acid residue linked in an α2-3 or α2-6 linkage to the non-reducing end of the galactose residue linked in a β1-4 linkage to the non-reducing end of the GlcNAc residue linked in a β1-4 linkage to the non-reducing end of the α1-3-linked mannose residue; (v) a sialic acid residue linked in an α2-3 or α2-6 linkage to the non-reducing end of the galactose residue linked in a β1-4 linkage to the non-reducing end of the GlcNAc residue linked in a β1-6 linkage to the non-reducing end of the α1-6-linked mannose residue; or (v) a combination of two or more selected from the group consisting of (i), (ii), (iii), and (iv).

The present invention further provides a method for remodeling the N-glycans of a composition of N-glycosylated glycoproteins, the steps comprising: (a) providing an aqueous composition of N-glycosylated glycoproteins: (b) immobilizing the N-glycosylated glycoproteins on a solid support: (c) reacting the immobilized N-glycosylated glycoproteins with a glycosylation enzyme or sequentially with two or more glycosylation enzymes to produce immobilized N-glycosylated glycoproteins comprising remodeled N-glycans; and (d) eluting the N-glycosylated glycoproteins comprising the remodeled N-glycans from the solid support to provide a composition of the N-glycosylated glycoproteins comprising remodeled N-glycans.

In a further embodiment, the solid support comprises irreversibly affixed thereon a multiplicity of capture moieties that specifically bind the N-glycosylated glycoprotein.

In a further embodiment, the N-glycosylated glycoprotein comprises an antibody or antigen binding fragment.

In a further embodiment, the multiplicity of capture moieties comprises protein A and the N-glycosylated glycoprotein comprises an antibody.

In a further embodiment, the one or more glycosylation enzymes are selected from the group consisting of exoglycosidases, endoglycosidases, and glycosyltransferases.

In a further embodiment, the immobilized N-glycosylated glycoproteins are reacted sequentially with two or more exoglycosidases selected from the group consisting neuraminidases, galactosidases, and an N-acetylglucosaminidases (GlcNAcases) to produce a population of N-glycosylated glycoproteins comprising remodeled N-glycans comprising Man₃GlcNAc₂ N-glycans having the structure

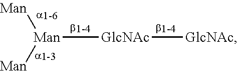

wherein the GlcNAc residue at the reducing end of the Man₃GlcNAc₂ N-glycans is linked to an asparagine residue forming an N-glycosylation site in the N-glycosylated glycoprotein, wherein Man is mannose and GlcNAc is N-acetylglucosamine.

In a further embodiment, the immobilized N-glycosylated glycoproteins are reacted sequentially with a neuraminidase, a galactosidase, and a GlcNAcase to produce a population of N-glycosylated glycoproteins comprising remodeled N-glycans comprising Man₃GlcNAc₂ N-glycans, wherein the GlcNAc residue at the reducing end of the Man₃GlcNAc₂ N-glycans is linked to an asparagine residue forming an N-glycosylation site in the N-glycosylated glycoprotein.

In a further embodiment, wherein the immobilized N-glycosylated glycoproteins comprising the Man₃GlcNAc₂ N-glycans are reacted with an N-acetylglucosaminyltransferase (GnT) I in the presence of UDP-GlcNAc to produce a population of N-glycosylated glycoproteins comprising remodeled N-glycans in which the terminal mannose residue of the α1-3 arm is further linked to a GlcNAc residue to produce hybrid GlcNAcMan₃GlcNAc₂ N-glycans having the structure

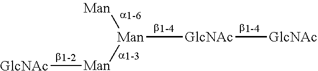

In a further embodiment, the immobilized N-glycosylated glycoproteins comprising the hybrid GlcNAcMan₃GlcNAc₂ N-glycans are reacted with a β1-4 galactosyltransferase (GalT) in the presence of UDP-galactose to produce a population of N-glycosylated glycoproteins comprising remodeled N-glycans in which the terminal GlcNAc residue of the α1-3 arm is further linked to a galactose residue (Gal) to produce hybrid GalGlcNAcMan₃GlcNAc₂ N-glycans having the structure

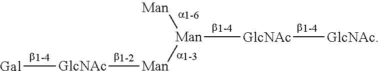

In a further embodiment, the immobilized N-glycosylated glycoproteins comprising the hybrid GalGlcNAcMan₃GlcNAc₂ N-glycans are reacted with an α2-6 sialyltransferase (SiaT) in the presence of CMP-sialic acid to produce a population of N-glycosylated glycoproteins comprising remodeled N-glycans in which the terminal galactose residue of the α1-3 arm is further linked to a sialic acid residue (Sia) to produce hybrid SiaGalGlcNAcMan₃GlcNAc₂ N-glycans having the structure

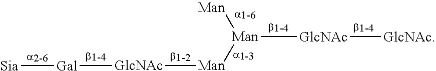

In a further embodiment, the immobilized N-glycosylated glycoproteins comprising the hybrid GalGlcNAcMan₃GlcNAc₂ N-glycans are reacted with an α2-3 sialyltransferase (SiaT) in the presence of CMP-sialic acid to produce a population of N-glycosylated glycoproteins comprising remodeled N-glycans in which the terminal galactose residue of the α1-3 arm is further linked to a sialic acid residue (Sia) to produce hybrid SiaGalGlcNAcMan₃GlcNAc₂ N-glycans having the structure

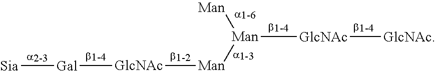

In a further embodiment, the immobilized N-glycosylated glycoproteins comprising the hybrid GlcNAcMan₃GlcNAc₂ N-glycans are reacted with a GnTII in the presence of UDP-GlcNAc to produce a population of N-glycosylated glycoproteins comprising remodeled N-glycans in which the terminal mannose residue of the α1-6 arm is further linked to a GlcNAc residue to produce bi-antennary GlcNAc₂Man₃GlcNAc₂ N-glycans having the structure

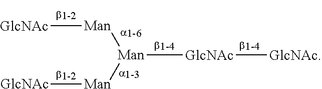

In a further embodiment, the immobilized N-glycosylated glycoproteins comprising the bi-antennary GlcNAc₂Man₃GlcNAc₂ N-glycans are reacted with a β1-4 galactosyltransferase (GalT) in the presence of UDP-galactose to produce a population of N-glycosylated glycoproteins comprising remodeled N-glycans in which the terminal GlcNAc residue of the α1-3 arm and/or terminal GlcNAc residue of the α1-6 arm is further linked to a galactose residue (Gal) to produce bi-antennary GalGlcNAc₂Man₃GlcNAc₂ N-glycans having the structure

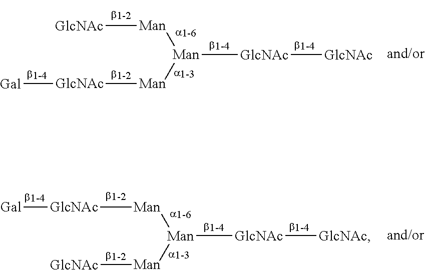

bi-antennary Gal₂GlcNAc₂Man₃GlcNAc₂N-glycans having the structure

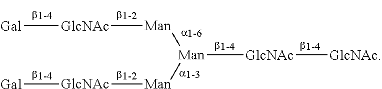

In a further embodiment, the immobilized N-glycosylated glycoproteins comprising the bi-antennary GalGlcNAc₂Man₃GlcNAc₂ and/or bi-antennary Gal₂GlcNAc₂Man₃GlcNAc₂ N-glycans are reacted with an α2-6 sialyltransferase (SiaT) in the presence of CMP-sialic acid to produce a population of N-glycosylated glycoproteins comprising remodeled N-glycans in which the terminal galactose residue of the α1-3 arm and/or terminal galactose residue of the α1-6 arm is further linked to a sialic acid residue (Sia) to produce bi-antennary SiaGal₂GlcNAc₂Man₃GlcNAc₂ N-glycans having the structure

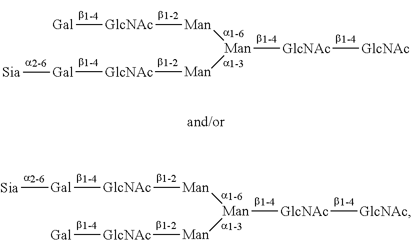

and/or bi-antennary Sia₂Gal₂GlcNAc₂Man₃GlcNAc₂N-glycans having the structure

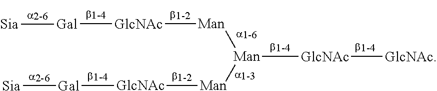

In a further embodiment, the immobilized N-glycosylated glycoproteins comprising the bi-antennary GalGlcNAc₂Man₃GlcNAc₂ and/or bi-antennary Gal₂GlcNAc₂Man₃GlcNAc₂ N-glycans are reacted with an α2-3 sialyltransferase (SiaT) in the presence of CMP-sialic acid to produce a population of N-glycosylated glycoproteins comprising remodeled N-glycans in which the terminal galactose residue of the α1-3 arm and/or terminal galactose residue of the α1-6 arm is further linked to sialic acid residue (Sia) to produce bi-antennary SiaGal₂GlcNAc₂Man₃GlcNAc₂ N-glycans having the structure

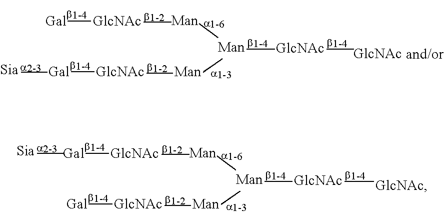

and/or bi-antennary Sia₂Gal₂GlcNAc₂Man₃GlcNAc₂ N-glycans having the structure

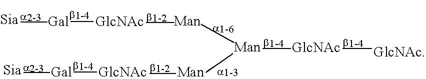

In a further embodiment, the immobilized N-glycosylated glycoproteins comprising the bi-antennary GlcNAc₂Man₃GlcNAc₂ N-glycans are reacted with a GnTIII in the presence of UDP-GlcNAc to produce a population of N-glycosylated glycoproteins comprising remodeled N-glycans in which the central mannose residue is further linked to a GlcNAc residue to produce bisected GlcNAc₃Man₃GlcNAc₂ N-glycans having the structure

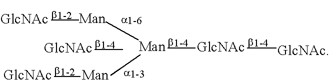

In a further embodiment, the immobilized N-glycosylated glycoproteins comprising the bi-antennary GlcNAc₃Man₃GlcNAc₂ N-glycans are reacted with a β1-4 galactosyltransferase (GalT) in the presence of UDP-glucose to produce a population of N-glycosylated glycoproteins comprising remodeled N-glycans in which the terminal GlcNAc residue of the α1-3 arm and/or terminal GlcNAc residue of the α1-6 arm is further linked to a galactose residue (Gal) to produce bi-antennary GalGlcNAc₃Man₃GlcNAc₂ N-glycans having the structure

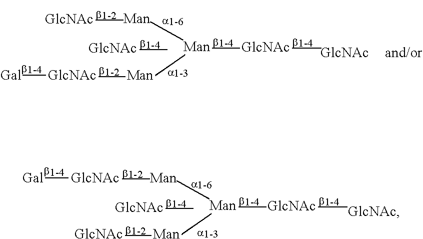

and/or bi-antennary Gal₂GlcNAc₃Man₃GlcNAc₂N-glycans having the structure

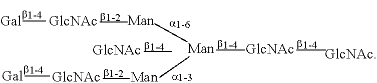

In a further embodiment, the immobilized N-glycosylated glycoproteins comprising the bi-antennary GalGlcNAc₃Man₃GlcNAc₂ and/or bi-antennary Gal₂GlcNAc₂Man₃GlcNAc₂ N-glycans are reacted with an α2-6 sialyltransferase (SiaT) in the presence of CMP-sialic acid to produce a population of N-glycosylated glycoproteins comprising remodeled N-glycans in which the terminal galactose residue of the α1-3 arm and/or terminal galactose residue of the α1-6 arm is further linked to a sialic acid residue (Sia) to produce bi-antennary SiaGal₂GlcNAc₃Man₃GlcNAc₂ N-glycans having the structure

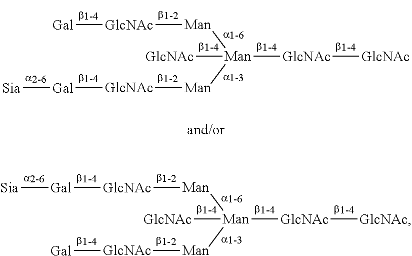

and/or bi-antennary Sia₂Gal₂GlcNAc₃Man₃GlcNAc₂N-glycans having the structure

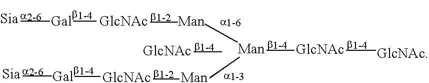

In a further embodiment, the immobilized N-glycosylated glycoproteins comprising the bi-antennary GalGlcNAc₃Man₃GlcNAc₂ and/or bi-antennary Gal₂GlcNAc₃Man₃GlcNAc₂ N-glycans are reacted with an α2-3 sialyltransferase (SiaT) in the presence of CMP-sialic acid to produce a population of N-glycosylated glycoproteins comprising remodeled N-glycans in which the terminal galactose residue of the α1-3 arm and/or terminal galactose residue of the α1-6 arm is further linked to sialic acid residue (Sia) to produce bi-antennary SiaGal₂GlcNAc₃Man₃GlcNAc₂ N-glycans having the structure

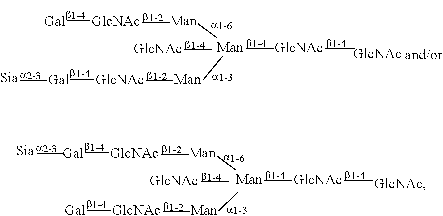

and/or bi-antennary Sia₂Gal₂GlcNAc₃Man₃GlcNAc₂ N-glycans having the structure

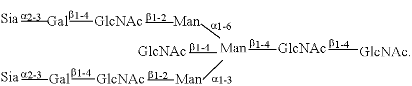

In a further embodiment, the immobilized N-glycosylated glycoproteins are reacted with one exoglycosidase or sequentially with two or more exoglycosidases to produce a population of N-glycosylated glycoproteins comprising remodeled N-glycans comprising GlcNAc₂Man₃GlcNAc₂ N-glycans having the structure

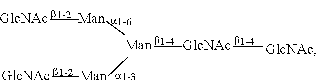

wherein the GlcNAc residue at the reducing end of the GlcNAc₂Man₃GlcNAc₂ N-glycans is linked to an asparagine residue forming an N-glycosylation site in the N-glycosylated glycoprotein, wherein Man is mannose and GlcNAc is N-acetylglucosamine.

In a further embodiment, the immobilized N-glycosylated glycoproteins are reacted sequentially with a neuraminidase and a galactosidase to produce a population of glycoproteins comprising remodeled N-glycans comprising the GlcNAc₂Man₃GlcNAc₂ N-glycans, wherein the GlcNAc residue at the reducing end of the GlcNAc₂Man₃GlcNAc₂ N-glycans is linked to an asparagine residue forming an N-glycosylation site in the N-glycosylated glycoprotein.

In a further embodiment, the immobilized N-glycosylated glycoproteins comprising the bi-antennary GlcNAc₂Man₃GlcNAc₂ N-glycans are reacted with a β1-4 galactosyltransferase (GalT) in the presence of UDP-galactose to produce a population of N-glycosylated glycoproteins comprising remodeled N-glycans in which the terminal GlcNAc residue of the α1-3 arm and/or terminal GlcNAc residue of the α1-6 arm is further linked to a galactose residue (Gal) to produce bi-antennary GalGlcNAc₂Man₃GlcNAc₂ N-glycans having the structure

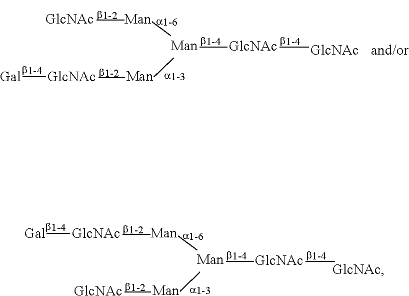

and/or bi-antennary Gal₂GlcNAc₂Man₃GlcNAc₂N-glycans having the structure

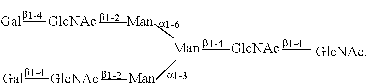

In a further embodiment, the immobilized N-glycosylated glycoproteins comprising the bi-antennary GalGlcNAc₂Man₃GlcNAc₂ and/or bi-antennary Gal₂GlcNAc₂Man₃GlcNAc₂ N-glycans are reacted with an α2-6 sialyltransferase (SiaT) in the presence of CMP-sialic acid to produce a population of N-glycosylated glycoproteins comprising remodeled N-glycans in which the terminal galactose residue (Gal) of the α1-3 arm and/or terminal galactose residue of the α1-6 arm is further linked to a sialic acid residue (Sia) to produce bi-antennary SiaGal₂GlcNAc₂Man₃GlcNAc₂ N-glycans having the structure

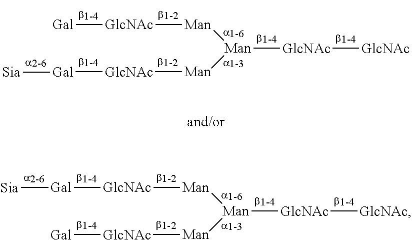

and/or bi-antennary Sia₂Gal₂GlcNAc₂Man₃GlcNAc₂N-glycans having the structure

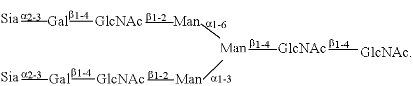

In a further embodiment, the immobilized N-glycosylated glycoproteins comprising the bi-antennary GalGlcNAc₂Man₃GlcNAc₂ and/or bi-antennary Gal₂GlcNAc₂Man₃GlcNAc₂ N-glycans are reacted with an α2-3 sialyltransferase (SiaT) in the presence of CMP-sialic acid to produce a population of N-glycosylated glycoproteins comprising remodeled N-glycans in which the terminal galactose residue of the α1-3 arm and/or terminal galactose residue of the α1-6 arm is further linked to a sialic acid residue (Sia) to produce bi-antennary SiaGal₂GlcNAc₂Man₃GlcNAc₂ N-glycans having the structure

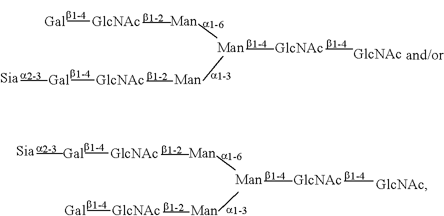

and/or bi-antennary Sia₂Gal₂GlcNAc₂Man₃GlcNAc₂ N-glycans having the structure

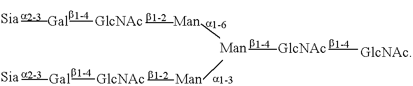

In a further embodiment, the immobilized N-glycosylated glycoproteins comprising the bi-antennary GlcNAc₂Man₃GlcNAc₂ N-glycans are reacted with a GnTIII in the presence of UDP-GlcNAc to produce a population of N-glycosylated glycoproteins comprising remodeled N-glycans in which the central mannose residue is further linked to a GlcNAc residue to produce bisected GlcNAc₃Man₃GlcNAc₂ N-glycans having the structure

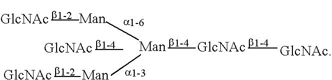

In a further embodiment, the immobilized N-glycosylated glycoproteins comprising the bisected GlcNAc₃Man₃GlcNAc₂ N-glycans are reacted with a β1-4 galactosyltransferase (GalT) in the presence of UDP-galactose to produce a population of N-glycosylated glycoproteins comprising remodeled N-glycans in which the terminal GlcNAc residue of the α1-3 arm and/or terminal GlcNAc residue of the α1-6 arm is further linked to a galactose residue (Gal) to produce bisected GalGlcNAc₃Man₃GlcNAc₂ N-glycans having the structure

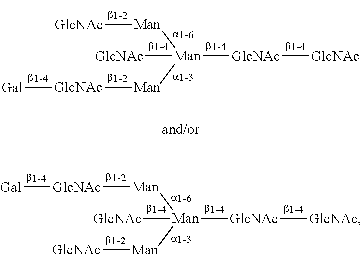

and/or bisected Gal₂GlcNAc₃Man₃GlcNAc₂N-glycans having the structure

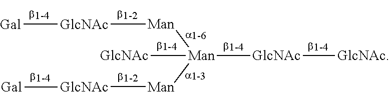

In a further embodiment, the immobilized N-glycosylated glycoproteins comprising the bisected GalGlcNAc₃Man₃GlcNAc₂ and/or bisected Gal₂GlcNAc₂Man₃GlcNAc₂ N-glycans are reacted with an α2-6 sialyltransferase (SiaT) in the presence of CMP-sialic acid to produce a population of N-glycosylated glycoproteins comprising remodeled N-glycans in which the terminal galactose residue of the α1-3 arm and/or terminal galactose residue of the α1-6 arm is further linked to a sialic acid residue (Sia) to produce bisected SiaGal₂GlcNAc₃Man₃GlcNAc₂ N-glycans having the structure

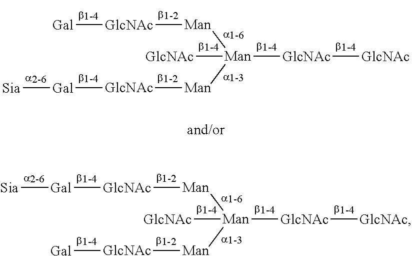

and/or bisected Sia₂Gal₂GlcNAc₃Man₃GlcNAc₂N-glycans having the structure

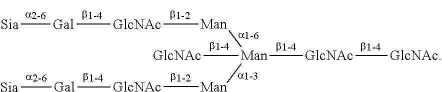

In a further embodiment, the immobilized N-glycosylated glycoproteins comprising the bisected GalGlcNAc₃Man₃GlcNAc₂ and/or bisected Gal₂GlcNAc₃Man₃GlcNAc₂ N-glycans are reacted with an α2-3 sialyltransferase (SiaT) in the presence of CMP-sialic acid to produce a population of N-glycosylated glycoproteins comprising remodeled N-glycans in which the terminal galactose residue of the α1-3 arm and/or terminal galactose residue of the α1-6 arm is further linked to a sialic acid residue (Sia) to produce bisected SiaGal₂GlcNAc₃Man₃GlcNAc₂ N-glycans having the structure

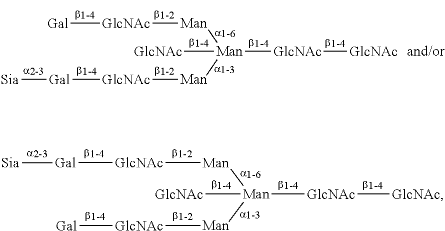

and/or bisected Sia₂Gal₂GlcNAc₃Man₃GlcNAc₂ N-glycans having the structure

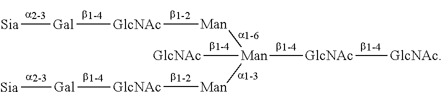

In a further embodiment, the immobilized N-glycosylated glycoproteins comprising the bi-antennary GlcNAc₂Man₃GlcNAc₂ N-glycans are reacted with a GnTIV in the presence of UDP-GlcNAc to produce a population of N-glycosylated glycoproteins comprising remodeled N-glycans in which the mannose residue of the α1-3 arm is further linked to a GlcNAc residue to produce α1-3 arm tri-antennary GlcNAc₃Man₃GlcNAc₂ N-glycans having the structure

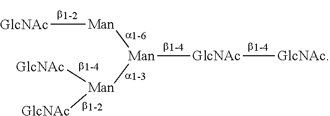

In a further embodiment, the immobilized N-glycosylated glycoproteins comprising the α1-3 arm tri-antennary GlcNAc₃Man₃GlcNAc₂ N-glycans are reacted with a β1-4 galactosyltransferase (GalT) in the presence of UDP-galactose to produce a population of N-glycosylated glycoproteins comprising remodeled N-glycans in which one, two, or all three of the terminal GlcNAc residues is further linked to a galactose residue (Gal) to produce α1-3 tri-antennary Gal₍₁₋₃₎GlcNAc₃Man₃GlcNAc₂ N-glycans, which structure represents that any one or two terminal GlcNAc residues are each linked to galactose, or all three terminal GlcNAc residues are each linked to galactose.

In a further embodiment, the immobilized N-glycosylated glycoproteins comprising the α1-3 arm tri-antennary Gal₍₁₋₃₎GlcNAc₃Man₃GlcNAc₂ N-glycans are reacted with an α2-6-sialyltransferase (α2-6-SiaT) or α2-3 sialyltransferase (α2-3-SiaT) in the presence of CMP-sialic acid to produce a population of N-glycosylated glycoproteins comprising remodeled N-glycans in which one, two, or all three of the terminal galactose residues is/are further linked to a sialic acid residue (Sia) to produce α2-6-Sia, α1-6 arm tri-antennary Sia₍₁₋₃₎Gal₍₁₋₃₎GlcNAc₃Man₃GlcNAc₂ N-glycans or α2-3-Sia, α1-6 arm tri-antennary Sia₍₁₋₃₎Gal₍₁₋₃₎GlcNAc₃Man₃GlcNAc₂ N-glycans, respectively, which structure represents that any one or two terminal galactose residues are each linked to sialic acid, or all three terminal galactose residues are each linked to sialic acid.

In a further embodiment, the immobilized N-glycosylated glycoproteins comprising the bi-antennary GlcNAc₂Man₃GlcNAc₂ N-glycans are reacted with a GnTV in the presence of UDP-GlcNAc to produce a population of N-glycosylated glycoproteins comprising remodeled N-glycans in which the mannose residue of the α1-6 arm is linked to a GlcNAc residue to produce α1-6 arm tri-antennary GlcNAc₃Man₃GlcNAc₂ N-glycans having the structure

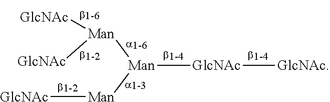

In a further embodiment, the immobilized N-glycosylated glycoproteins comprising the α1-6 arm tri-antennary GlcNAc₃Man₃GlcNAc₂ N-glycans are reacted with a β1-4 galactosyltransferase (GalT) in the presence of UDP-galactose to produce a population of N-glycosylated glycoproteins comprising remodeled N-glycans in which one, two, or all three of the terminal GlcNAc residues is/are further linked to a galactose residue (Gal) to produce α1-6 arm tri-antennary Gal₍₁₋₃₎GlcNAc₃Man₃GlcNAc₂ N-glycans, which structure represents any one or two terminal GlcNAc residues are each linked to galactose, or all three terminal GlcNAc residues are each linked to galactose.

In a further embodiment, the immobilized N-glycosylated glycoproteins comprising the α1-6 arm tri-antennary Gal₍₁₋₃₎GlcNAc₃Man₃GlcNAc₂ N-glycans are reacted with an α2-6-sialyltransferase (α2-6-SiaT) or α2-3 sialyltransferase (α2-3-SiaT) in the presence of CMP-sialic acid to produce a population of N-glycosylated glycoproteins comprising remodeled N-glycans in which one, two, or all three of the terminal galactose residues is/are further linked to a sialic acid residue (Sia) to produce α2-6-Sia, α1-6 arm tri-antennary Sia₍₁₋₃₎Gal₍₁₋₃₎GlcNAc₃Man₃GlcNAc₂ N-glycans or α2-3-Sia, α1-6 arm tri-antennary Sia₍₁₋₃₎Gal₍₁₋₃₎GlcNAc₃Man₃GlcNAc₂ N-glycans, respectively, which structure represents any one or two terminal galactose residues are each linked to sialic acid, or all three terminal galactose residues are each linked to sialic acid.

In a further embodiment, (i) the immobilized N-glycosylated glycoproteins comprising the α1-3 arm tri-antennary GlcNAc₃Man₃GlcNAc₂ N-glycans is further reacted with a GnTV in the presence of UDP-GlcNAc or (ii) the immobilized N-glycosylated glycoproteins comprising the α1-6 arm tri-antennary GlcNAc₃Man₃GlcNAc₂ N-glycans is further reacted with a GnTIV in the presence of UDP-GlcNAc, to produce a population of N-glycosylated glycoproteins comprising remodeled N-glycans in which the mannose residues of the α1-3 arm and α1-6 arm are each further linked to a GlcNAc residue to produce multi-antennary GlcNAc₄Man₃GlcNAc₂ N-glycans having the structure

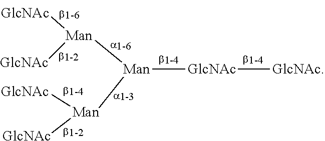

In a further embodiment, the immobilized N-glycosylated glycoproteins comprising the multi-antennary GlcNAc₄Man₃GlcNAc₂ N-glycans are reacted with a β1-4 galactosyltransferase (GalT) in the presence of UDP-galactose to produce a population of N-glycosylated glycoproteins comprising remodeled N-glycans in which one, two, three, or all four of the terminal GlcNAc residues is/are further linked to a galactose residue (Gal) to produce Gal₍₁₋₄₎GlcNAc₄Man₃GlcNAc₂ N-glycans, which structure represents any one, two, or three terminal GlcNAc residues are each linked to galactose, or all four terminal GlcNAc residues are each linked to galactose.

In a further embodiment, the immobilized N-glycosylated glycoproteins comprising the α1-6 arm multi-antennary Gal₍₁₋₄₎GlcNAc₄Man₃GlcNAc₂ N-glycans are reacted with an α2-6-sialyltransferase (α2-6-SiaT) or α2-3 sialyltransferase (α2-3-SiaT) in the presence of CMP-sialic acid to produce a population of N-glycosylated glycoproteins comprising remodeled N-glycans in which one, two, three, or all four of the terminal galactose residues is/are further linked to a sialic acid residue (Sia) to produce α2-6-Sia multi-antennary Sia₍₁₋₄₎Gal₍₁₋₄₎GlcNAc₄Man₃GlcNAc₂ N-glycans or α2-3-Sia multi-antennary Sia₍₁₋₄₎Gal₍₁₋₄₎GlcNAc₄Man₃GlcNAc₂ N-glycans, respectively, which structure represents any one, two, or three terminal galactose residues are each linked to sialic acid, or all four terminal galactose residues are each linked to sialic acid.

### BRIEF DESCRIPTION OF THE DRAWINGS

FIG. 1A. Scheme of solid-phase glycan remodeling (SPGR) protocols.

FIG. 1B. Enzyme map for N-glycan glycoengineering. Shown is a bi-antennary N-glycan.

FIG. 1C. Chromatogram of glycans collected from human serum IgG. The Oxford notation name is used for glycan nomenclature. The glycan structures are presented according to the Consortium for Functional Glycomics (CFG).

FIG. 1D. The functions of N-acetylglucosaminyltransferases (GnTs). Note that GnTIII inhibits the activity of GntTV and GnTV and vice versa.

FIG. 2. Chromatogram of glycans collected from IgG treated with different glycosylation enzymes. The data was collected from reactions that reached, or were close to, the plateau of the conversion. The formation of glycoforms was confirmed by mass spectrometry analyses. Refer to Table 3 for detailed reaction conditions. Star marks indicated the substrate glycan species that have not been fully transformed in the reaction.

FIG. 3A-C. Glycan chromatograms from IgGs after sequential glycoengineering using SPGR. FIG. 3A: Process of remodeling IgG glycans into core saccharides (FM3 and M3 glycoforms). FIG. 3B: Process of re-building core saccharides into hybrid species. About 10% (F)M3 glycans remained in the products due to the reversible activity of GnT-I. FIG. 3C: Process of re-building FA2 and A2 glycans into bisecting species. Refer to FIG. 4 for the sample numbering and Table 2 for the buffer conditions.

FIG. 4. Scheme of SPGR routes investigated in this work. Bracket: with or without the residues (micro-heterogeneity). Serum IgG contains about 5% defucosylated population and about 10% bisecting population. For simplicity, these two populations are not shown in the scheme. The conversion (CV) ratio was calculated based on the consumption of substrate glycan species.

FIG. 5A-D. Physical and biochemical characterization of SPGR-engineered IgGs.

FIG. 5A: Size analyses using dynamic light scattering (DLS). FIG. 5B: Melting temperature analyses (Tₘ). FIG. 5C: Aggregation temperature analyses (T_{agg}). Endo S treatment removed most IgG glycan structures, leaving only GlcNAc-Fuc di-saccharide on IgG. FIG. 5D: Competition assay revealed the binding affinity of SPGR-engineered IgG to Fc gamma receptor I (FcγR I). The lower the relative intensity, the stronger was the interaction between SPGR-engineered IgG and FcγR I. Refer to FIG. 3 and FIG. 6 for the chromatograms of analyzed samples.

FIG. 6A-B. Chromatogram of fluorescently labeled glycans collected from IgG undergoing successive glycan remodeling using SPGR. FIG. 6A: Harmonization of terminal residues into galactose. FIG. 6B: Harmonization of terminal residues into sialic acid. The reactions were conducted on 1 mg human serum IgG immobilized on 0.1 mL protein A resin. Refer to FIG. 4 for the sample numbering and Table 2 for working conditions.

FIG. 7A-C. Characterization of Neuraminidase (C. perfringens) activity and its working condition optimization. FIG. 7A: Temperature optimization. FIG. 7B: Dose-dependent experiment. FIG. 7C: Time-course study. Error bars: mean, maximum and minimum values. Refer to Table 3 for reaction conditions.

FIG. 8A-C. Characterization of Galactosidase S (S. pneumoniae) activity and its working condition optimization. FIG. 8A: Temperature optimization. FIG. 8B: Dose-dependent experiment. FIG. 8C: Time-course study. Error bars: mean, maximum and minimum values. Refer to Table 3 for reaction conditions.

FIG. 9A-C. Characterization of N-Acetylglucosaminidase S (S. pneumoniae) activity and its working condition optimization. FIG. 9A: Temperature optimization. FIG. 9B: Dose-dependent experiment. FIG. 9C: Time-course study. Error bars: mean, maximum and minimum values. Refer to Table 3 for reaction conditions.

FIG. 10A-D. Characterization of fucosidase (Candidatus Omnitrophica) activity on intact IgG. FIG. 10A: A three-day reaction with intact IgG bearing (F)M3 glycans revealed the activity of fucosidase. The (F)M3 glycoforms were prepared using SPGR. FIG. 10B: Comparison of conversion ratio between reactions with immobilized IgG and free IgG. FIG. 10C: A five-day reaction with intact IgG (no immobilization) revealed the broad substrate spectrum of the enzyme. Defucosylated populations were increased (highlighted with glycan images). FIG. 10D: Fucosidase activity decreased as the structural complexity of glycans increased. Refer to Table 3 for reaction conditions.

FIG. 11A-G. Characterization of α2-6 Sialyltransferase (H. sapiens) activity and its working condition optimization. FIG. 11A: Temperature optimization. FIG. 11B: pH optimization. FIG. 11C: Cation optimization. FIG. 11D: Dose-dependent experiment. FIG. 11E: Time-course study. Conversion ratio calculations for α2-6 Sialyltransferase reactions were based on the consumption of non-sialylated glycans with terminal galactoses. FIG. 11F: Comparison of enzyme activity between different IgG glycoforms. The data was collected from the time-course experiments. FIG. 11G: Comparison of enzyme activity between the installation of 1^{st} and 2^{nd} sialic acid. Refer to Table 3 for reaction conditions.

FIG. 12A-E. Characterization of β1-4 Galactosyltransferase 1 (H. sapiens) activity and its working condition optimization. FIG. 12A: Temperature optimization. FIG. 12B: pH optimization. FIG. 12C: Cation optimization. FIG. 12D: Dose-dependent experiment. FIG. 12E: Time-course study. Error bars: mean, maximum and minimum values. Refer to Table 3 for reaction conditions.

FIG. 13A-B. Characterization of N-Acetylglucosaminyltransferases I (GnT-I, MGAT1) activity. FIG. 13A: Chromatographic analyses revealed GnT-I activity on (F)M3 glycans prepared by SPGR. FIG. 13B: Mass spectrometry confirmed the formation of (F)A1[3] glycans. Glycan samples for the mass spectrometry analyses were labeled by RapiFlour-MS probe (MW=312.4) from Waters. On-column cleavage of terminal GlcNAc residues was often observed in HPLC-MS analyses. The IgG substrate bearing (F)M3 glycans was prepared by SPGR.

FIG. 14A-F. Characterization of N-Acetylglucosaminyltransferase 1 (GnT-I, H. sapiens) activity and its working condition optimization. FIG. 14A: Temperature optimization. FIG. 14B: pH optimization. FIG. 14C: Cation optimization. FIG. 14D: Dose-dependent experiment. FIG. 14E: Time-course study. FIG. 14F: The conversion ratio reached a plateau at about 85%. Error bars: mean, maximum and minimum values. Refer to Table 3 for reaction conditions.

FIG. 15A-F. Characterization of N-Acetylglucosaminyltransferase 3 (GnT-III, H. sapiens) activity and its working condition optimization. FIG. 15A: Temperature optimization. FIG. 15B: pH optimization. FIG. 15C: Cation optimization. FIG. 15D: Dose-dependent experiment. FIG. 15E: Time-course study. FIG. 15F: Comparison of enzyme activity (in conversion ratio) between different IgG glycoforms. The data was collected from the time-course experiments. Refer to Table 3 for reaction conditions.

FIG. 16A-C. In vitro activity of N-Acetylglucosaminyltransferase 5 (GnT-V, H. sapiens) on intact human serum IgG immobilized on protein A resin. FIG. 16A: GnT-V reaction with native human IgG. A new peak (indicated by arrowhead) was found after a 24 hour reaction. FIG. 16B: GnT-V reaction with glycoengineered human IgG. Human serum IgG was first treated with galactosidase in order to minimize the overlapping between the product signal and the FA2GI glycan signals. A signal increase in the newly found peak was observed when the incubation time was increased. FIG. 16C: Mass spectrometry analysis of the newly formed peak after the GnT-V reaction. The molecular weight (reported in m/z) of FA3 glycan was confirmed. The fragments resulting from on-column cleavage of GlcNA also supported that this analyte contains three terminal GlcNAc. The newly formed FA3 glycan has a one-minute shift in retention time from that of FA2B glycan, despite they share the same molecular weight. Glycan samples for the mass spectrometry analyses showing here were labeled by RapiFlour-MS probe from Waters (MW=312.4).

FIG. 17. Characterization of fucosyltransferase (Homo sapiens, FUT8) activity. Serum IgG was treated with fucosidase (100 mol %) for 5 days. The resulting product (30% of the IgG population was defucosylated) was then used for the FUT8 reaction. The arrows indicate the glycoforms consumed by FUT8. Refer to Table 3 for reaction conditions.

FIG. 18A-D. Investigating the binding affinity between SPGR-engineered IgG and Fcγ Receptor I using competition assays. IgG-FcRI interaction resulted in a signal decrease. Note that all the IgG samples (1-10) contained about 5% defucosylated population. The biantennary samples (2-4) had about 10% bisecting glycoforms; while the mono-antennary samples (5-7) also had about 10% (F)M3 glycans due to the GlcNAcase activity of GnT-I. Sialylated samples (4 & 10) possessed about 1:1 mono- and bi-sialylated populations. The Fuc-treated sample contained a 30% defucosylated glycan population. Effective concentration that leads to 50% signal reduction (EC₅₀) was calculated using IC₅₀ curve fitting by GraphPad Prism 8 (Dose-response, inhibition).

FIG. 19. Comparison between substrate immobilization and enzyme immobilization in SPGR.

FIG. 20. Characterization of endoglycosidase (Streptococcus pyogenes) activity and its working condition optimization. FIG. 20A: Chromatography of IgG glycans before and after endo S treatment. FIG. 20B: Temperature optimization. FIG. 20C: Dose-dependent experiment. FIG. 20D: Time-course study. Error bars: mean, maximum and minimum values. Refer to Table 3 for reaction conditions.

FIG. 21A. Comparison of enzyme activity between the use of free and immobilized IgG as substrates.

FIG. 21B-C. Computational modeling of IgG Fc region and protein A interaction.

FIG. 21B shows a Front-view and FIG. 21C shows a side-view of IgG Fc-protein A complex (two Fc regions, each bound to protein A). N-glycans at Asn297 are indicated (numbering according to Eu).

FIG. 22A-F. Representative structures of some of the N-glycans that may be made using the SPGR process.

### DETAILED DESCRIPTION OF THE INVENTION

### Definitions

As used herein, the Oxford notation name is used for glycan nomenclature and the glycan structures are presented according to the Consortium for Functional Glycomics (CFG). As used herein, the term “glycosylation enzymes” refers to exoglycosidases, endoglycosidases, and glycosyltransferases, and includes both native and naturally occurring enzymes and recombinant variants thereof that comprise at least the catalytic activity of the glycosylation enzyme.

As used herein, the term “exoglycosidases” are glycoside hydrolase enzymes (EC 3.2) that cleave the glycosidic linkage of a terminal monosaccharide in an oligosaccharide or polysaccharide. Exoglycosidases include neuraminidases, galactosidases, N-acetylglucosaminidases, and fucosidases.

As used herein, “endoglycosidases” are glycoside hydrolase enzymes (EC 3.2) that cleave the glycosidic linkage between the two N-acetylglucosamine (GlcNAc) residues of the chitobiose core, but which recognize and cleave different types of N-linked glycans. For example, endoglycosidase H cleaves within the chitobiose core of high mannose and some hybrid oligosaccharides from N-linked glycoproteins, endoglycosidase S is highly specific for removing N-linked glycans from the heavy chain of native IgG, and endoglycosidase D cleaves paucimannose N-linked glycans.

As used herein, “glycosyltransferases” are enzymes (EC 2.4) that catalyze the transfer of saccharide moieties from an activated nucleotide sugar (also known as the “glycosyl donor”) to a nucleophilic glycosyl acceptor molecule, the nucleophile of which can be oxygen-carbon-, nitrogen-, or sulfur-based. Examples of glycosyltransferases include but are not limited to N-acetylglucosaminyltransferase (GnT) I, which transfers GlcNAc from UDP-GlcNAc to the α1,3-linked terminal mannose (Man) residue on paucimannose N-glycans; GnTII, which transfers GlcNAc from UDP-GlcNAc to the α1,6-linked terminal mannose of GlcNAcMan₃GlcNAc₂ N-glycans in a β1-2 linkage; GnTIII, which transfers GlcNAc from UDP-GlcNAc to the α1,4-linked central mannose of GlcNAcMan₃GlcNAc₂ N-glycans in a β1-4 linkage; β1,4-galactosyltransferase (GalT), which transfers galactose (Gal) from UDP-galactose to the terminal GlcNAc residues of N-glycans in a β1-4 linkage; α2,3-sialyltransferase (α2,3-sialyIT) or α2,6-sialyltransferase (α2,6-sialyIT), which transfers sialic acid from CMP-sialic acid to the terminal galactose residues of N-glycans in an α2-6 linkage; α2,3-sialyltransferase (α2,3-sialyIT), which transfers sialic acid from CMP-sialic acid to the terminal galactose residues of N-glycans in an α2-3 linkage; and, α1,6-fucosyltransferase (FUT8) transfers fucose from GDP-fucose to the GlcNAc residue at the reducing end of the chitobiose core in an α1,6 linkage.

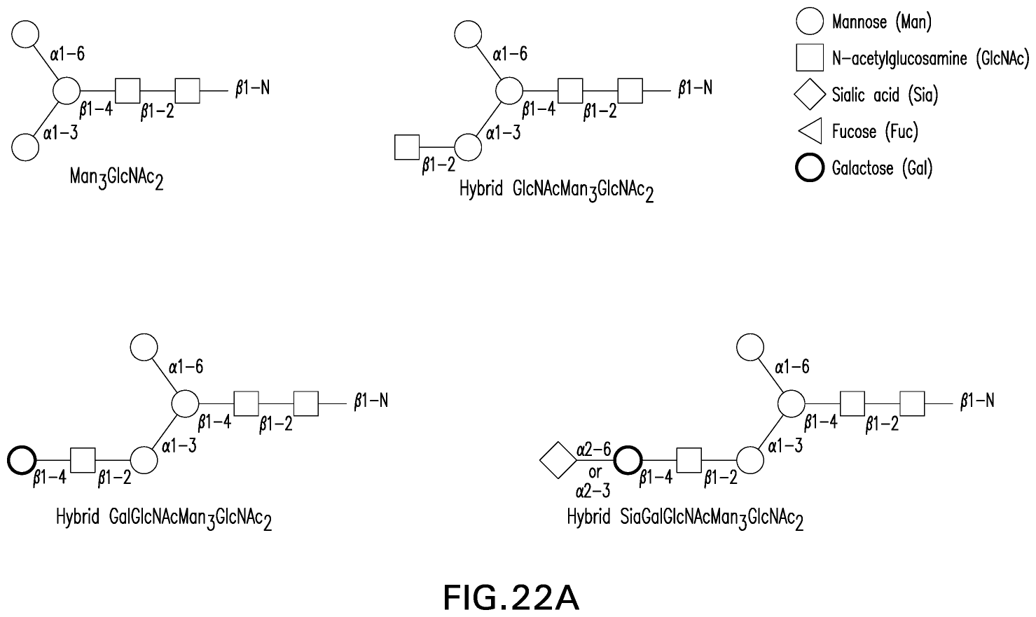

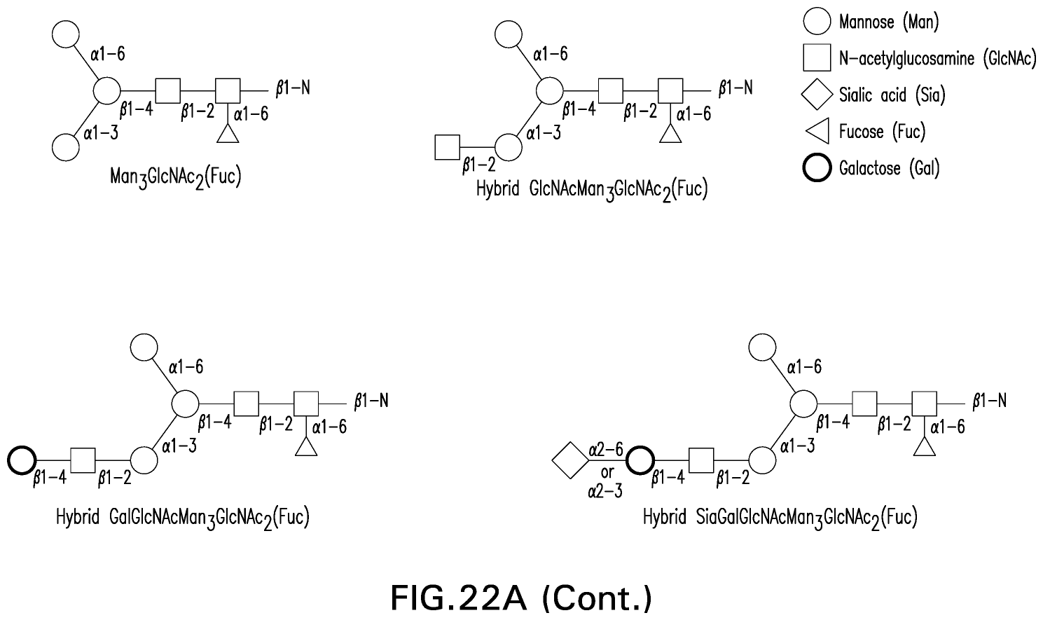

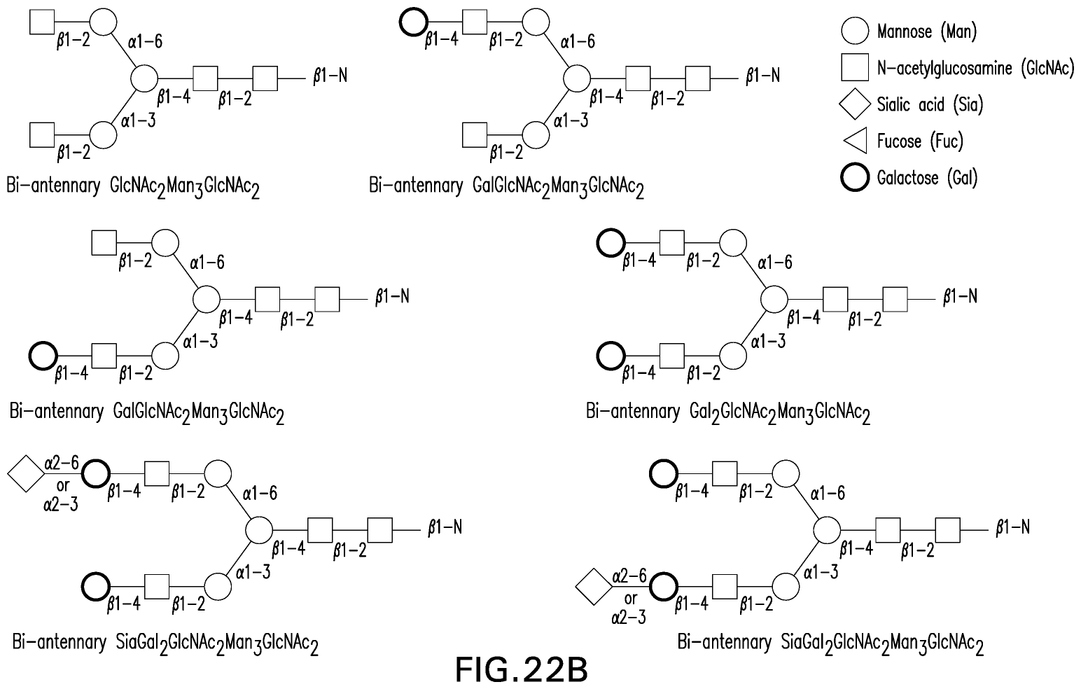

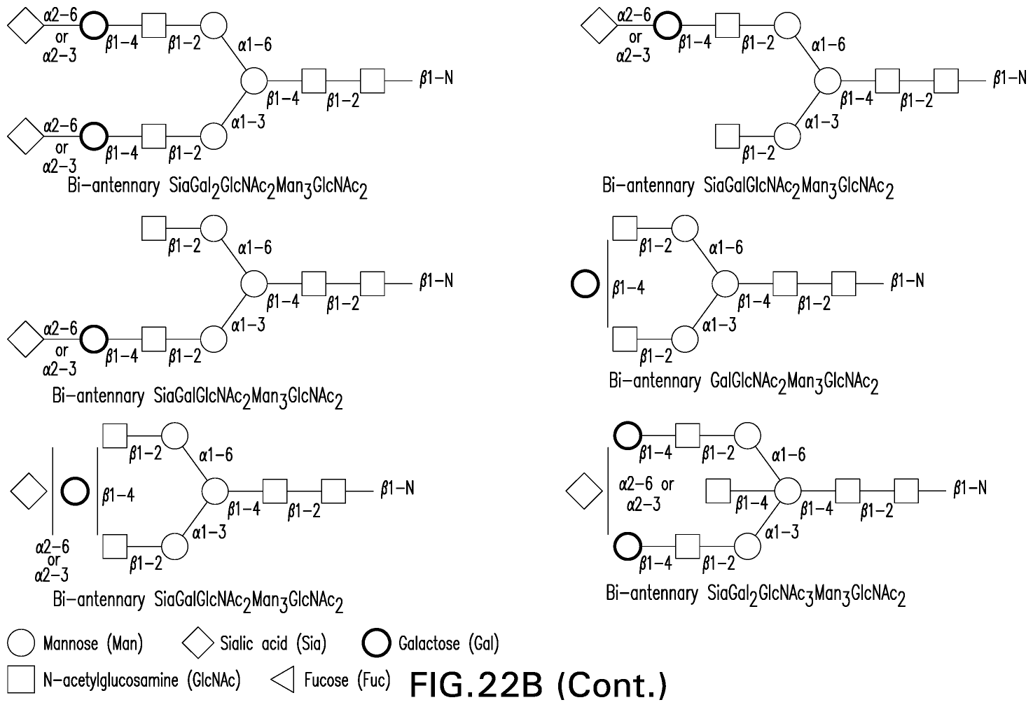

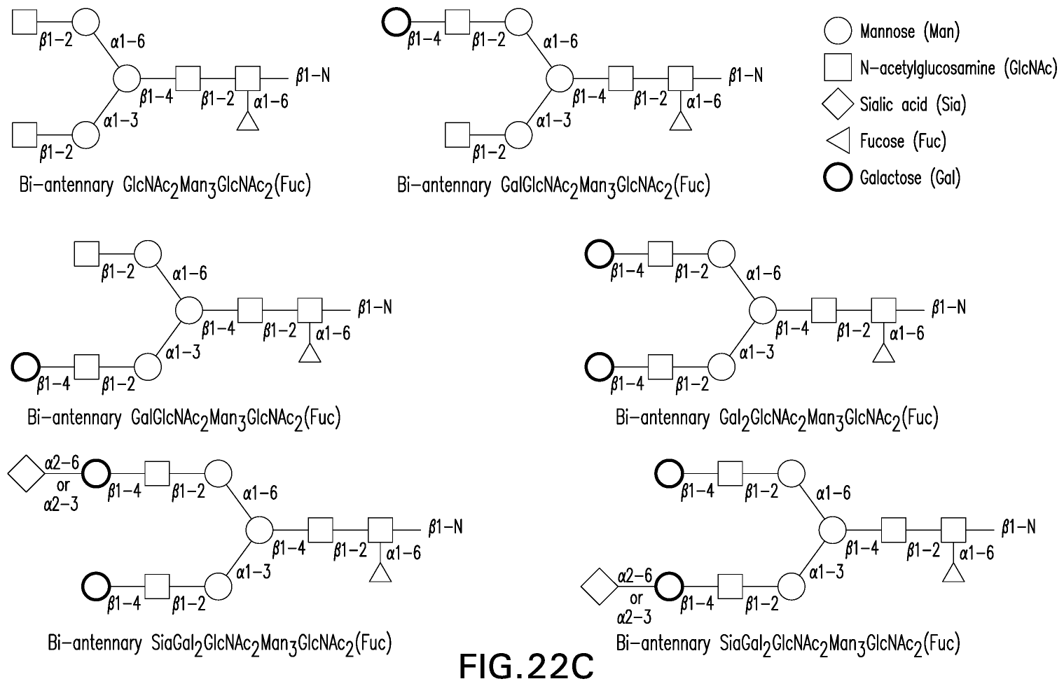

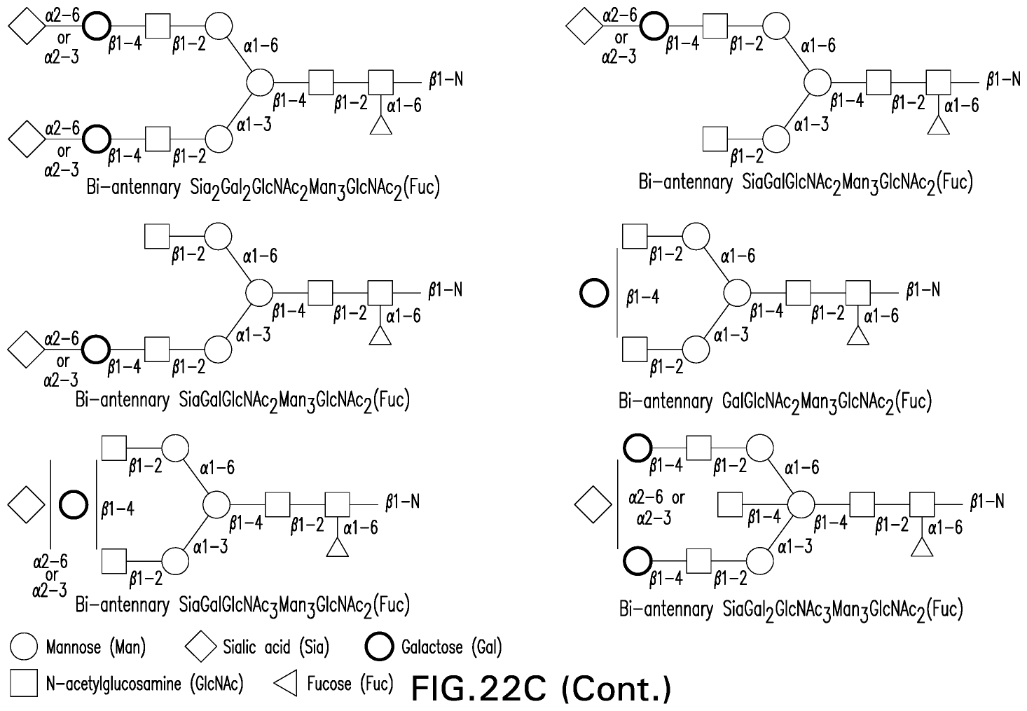

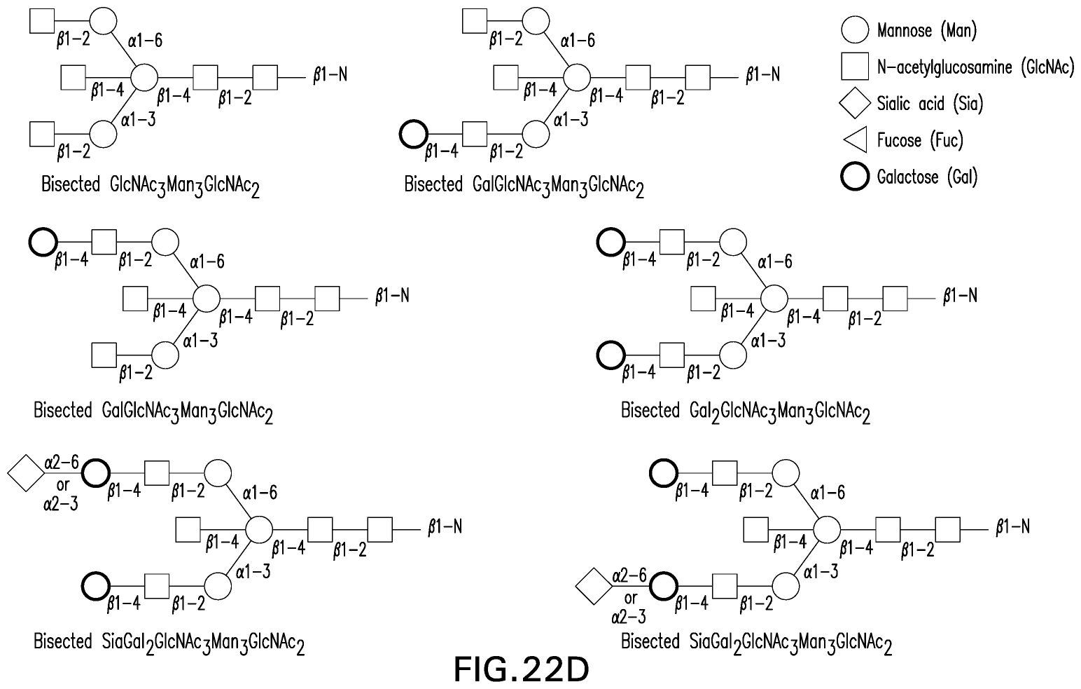

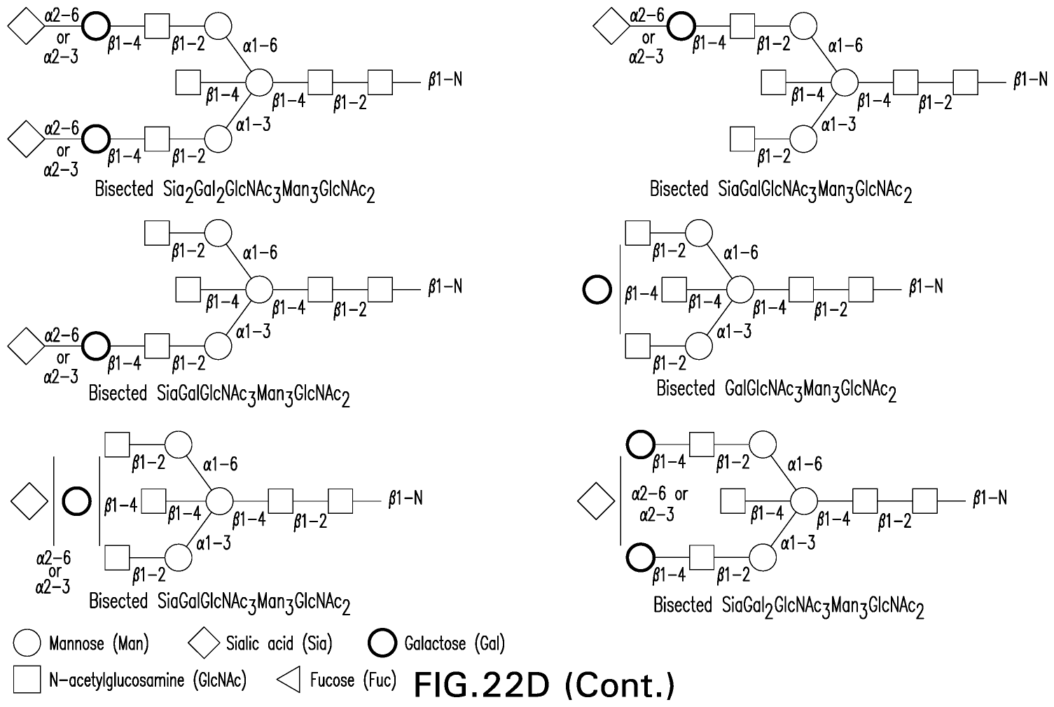

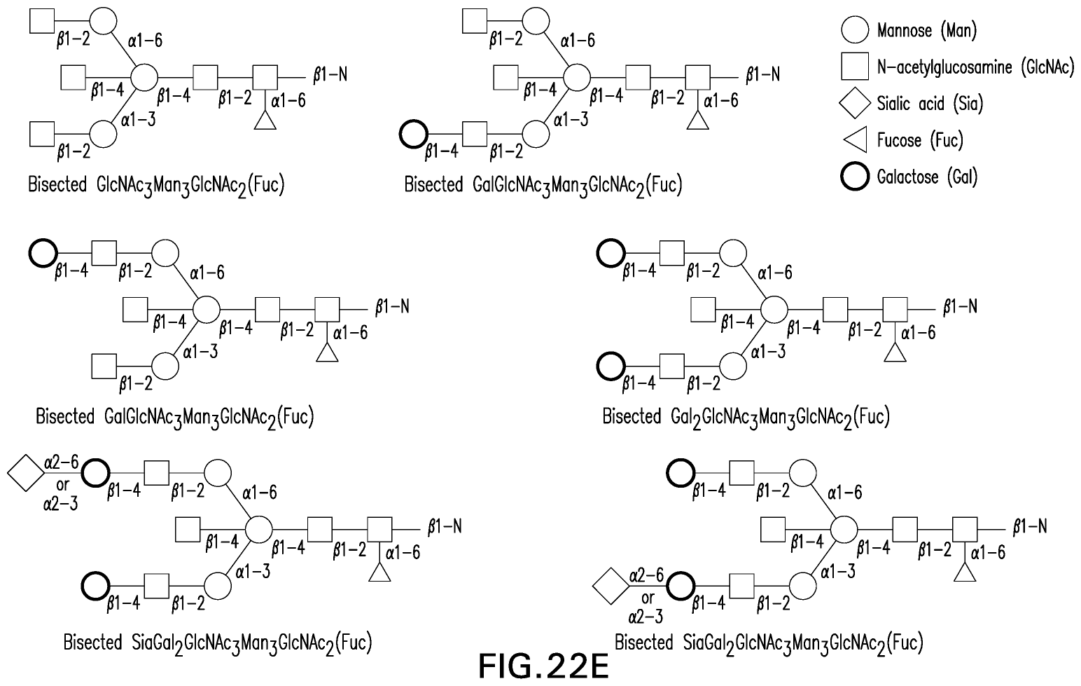

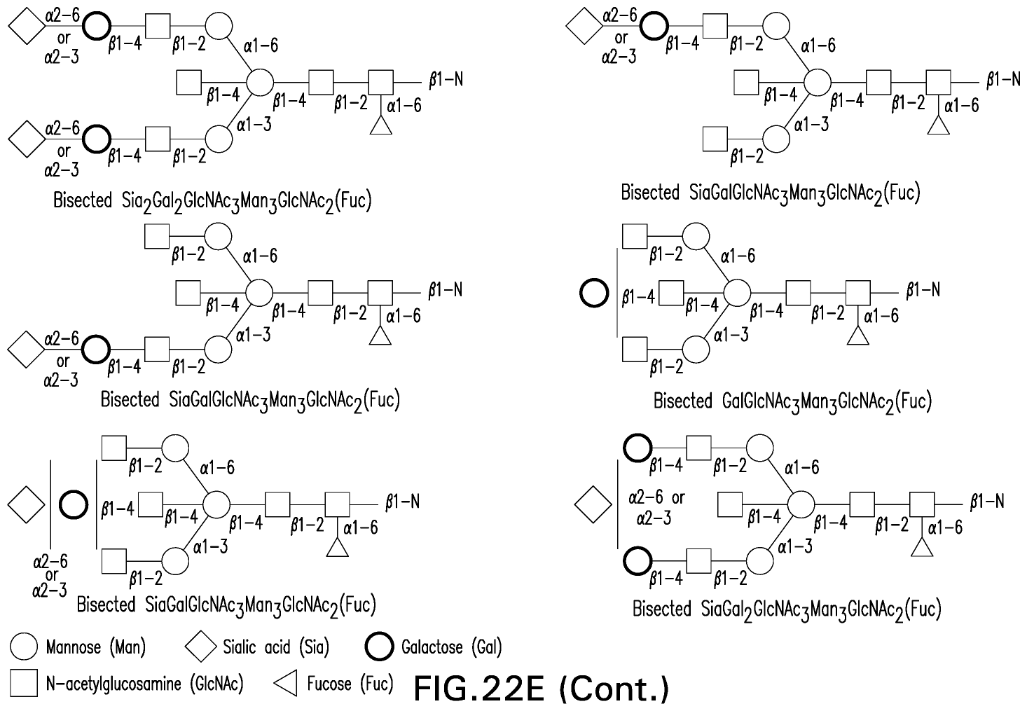

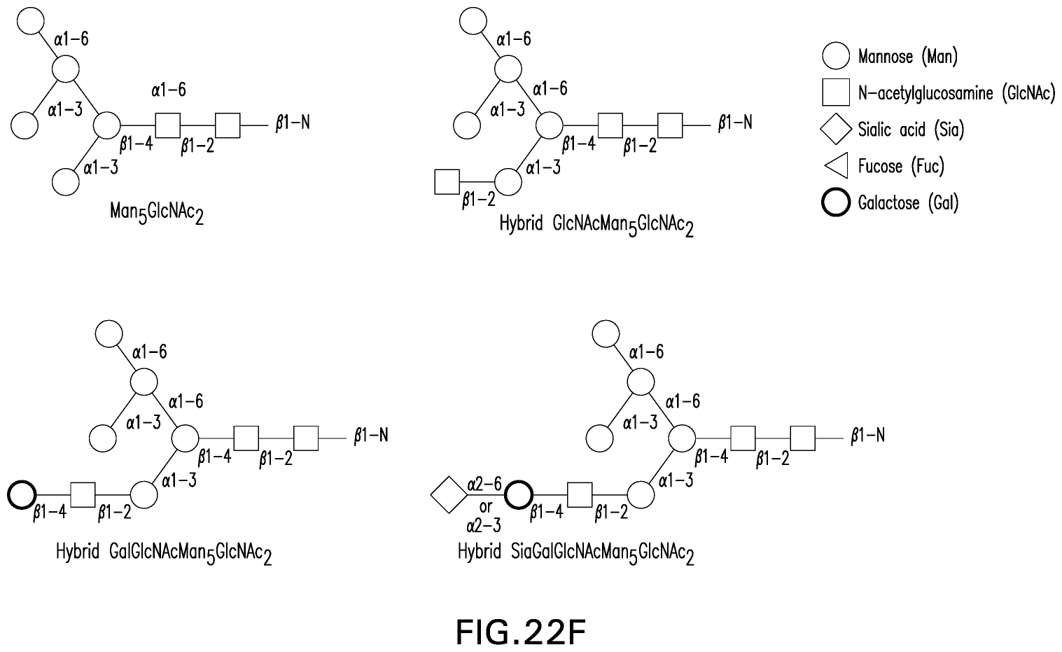

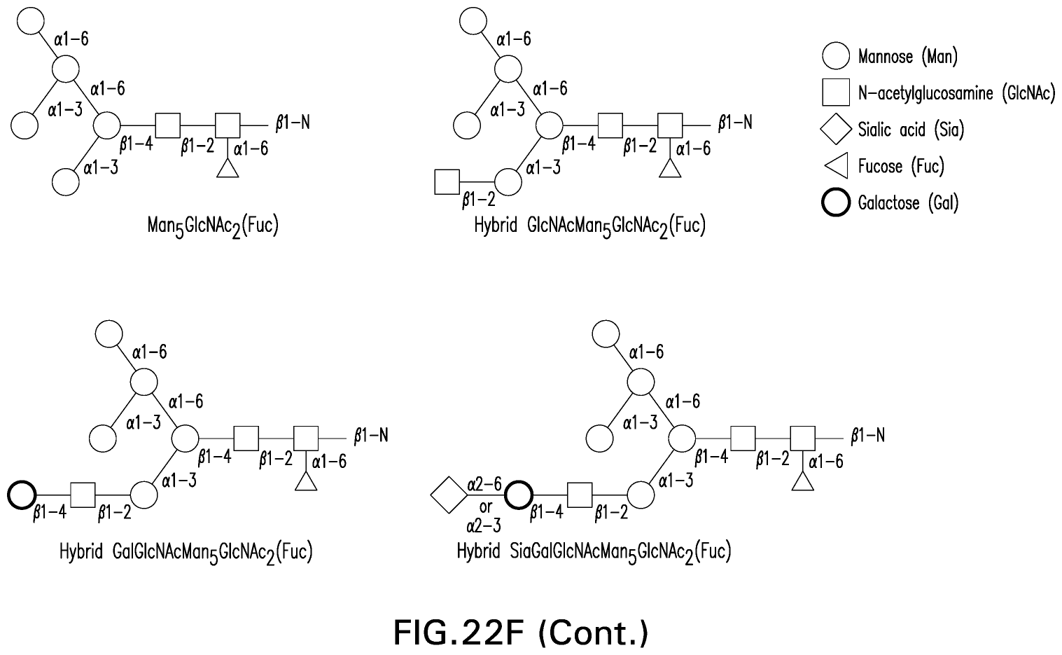

As used herein, the term “N-glycan” refers to an N-linked oligosaccharide, e.g., one that is attached by an asparagine-N-acetylglucosamine linkage to an asparagine residue of a polypeptide. N-glycans have a common pentasaccharide core of Man₃GlcNAc₂ (“Man” refers to mannose: “Glc” refers to glucose; and “NAc” refers to N-acetyl; GlcNAc refers to N-acetylglucosamine). The term “trimannose core” or “paucimannose core” used with respect to the N-glycan also refers to the structure Man₃GlcNAc₂ (“Man₃”). N-glycans differ with respect to the number of branches (antennae) comprising peripheral sugars (e.g., fucose and sialic acid) that are added to the Man₃ core structure. N-glycans are classified according to their branched constituents (e.g., high mannose, complex or hybrid). FIG. 22A-22F shows the structures of a representative group of N-glycans that may be produced using the SPGR process.

A “high mannose” type N-glycan has five or more mannose residues. A “complex” type N-glycan typically has at least one GlcNAc attached to the α1,3 mannose arm and at least one GlcNAc attached to the α1,6 mannose arm of the trimannose core. Complex N-glycans may also have galactose (“Gal”) residues that are optionally modified with sialic acid or derivatives (“NeuAc”, where “Neu” refers to neuraminic acid and “Ac” refers to acetyl). Complex N-glycans may also have intrachain substitutions comprising “bisecting” GlcNAc and core fucose (“Fuc”). A “hybrid” N-glycan has at least one GlcNAc on the terminal of the α1,3 mannose arm of the trimannose core and zero or more mannoses on the α1,6 mannose arm of the trimannose core.

As used herein, the term “predominant” or “predominantly” used with respect to the production of N-glycans refers to a structure which represents the major peak detected by ultrahigh pressure liquid chromatography with ultraviolet (UPLC-UV) absorbance measurement. Glycan masses were confirmed with UPLC interfaced to electrospray quadrupole time-of-flight mass spectrometry (ESI-QTOF-MS).

As used herein, the term “multiplicity” refers to a large number of the same or substantially same molecules, proteins, enzymes, species, etc. For example, a multiplicity of glycoproteins refers to a population of glycoproteins in which the glycoproteins have the same amino acid sequence and the same N-glycan structure. For example, a multiplicity of glycoproteins refers to a population of glycoproteins wherein the glycoproteins have substantially the same amino acid sequence and the N-glycans have substantially the same structure.

As used herein, the term “plurality” refers to a large number of a variety of species of molecules, proteins, enzymes, species, etc. For example, a plurality of glycoproteins refers to (i) a population of glycoproteins in which the glycoproteins have substantially the same amino acid sequence and more than one N-glycan structure. e.g., N-glycans comprising a mixture of Sia₍₁₋₂₎Gal₍₁₋₂₎GlcNAc₍₁₋₂₎Man₃GclNAc₂, Gal₍₁₋₂₎GlcNAc₍₁₋₂₎Man₃GclNAc₂, and GlcNAc₍₁₋₂₎Man₃GclNAc₂ structures, or (ii) a population of glycoproteins in which the glycoproteins comprise more than one amino acid sequence and a single N-glycan structure or more than one N-glycan structure.

As used herein, the term “sequentially” refers to forming or following in a logical order or sequence. For example, a glycoprotein may be reacted with a neuraminidase, a galactosidase, and a GlcNAcase in that order to produce a paucimannose N-glycan.

As used herein, the term “remodel” or “remodeling” refers to changing the structure of an N-glycan having a particular glycoform to another glycoform, by removing and/or adding sugar residues to the N-glycan. The term further includes changing the structures of the N-glycans of a population of glycoproteins.

Abbreviations used herein are of common usage in the art, see, e.g., abbreviations of sugars, above.

### The Solid-Phase Glycan Remodeling System

The present invention provides a solid-phase glycan remodeling (“SPGR”) system wherein glycosylation enzymatic reactions are carried out on glycoprotein substrates immobilized on solid supports. The SPGR system enables efficient reaction swapping, substrate purification, and the recovery of both remodeled glycosylated glycoprotein products and the glycosylation engineering enzymes. The SPGR system is particularly useful for glycoengineering immunoglobulins (antibodies or IgG) to provide antibodies that have predominantly particular glycoform profiles. The SPGR system enables harmonization of antibody glycans into one or more of ten different glycoforms, including non-canonical structures, in 48 hours with an averaged conversion ratio greater than 95%. Physical and biochemical analyses indicate that the SPGR-engineered antibodies preserved integrity and functionality, suggesting that the SPGR system has high biocompatibility to the antibody substrates.

For decades there has been a clear demand for glycoengineering tools that enable the production of glycoproteins having particular glycoforms for therapeutic uses as well as evaluating the role(s) of glycan structures on the function and form of glycoconjugates. SPGR of the present invention presents a straightforward strategy for controlling glycan structures with several advantages: 1) it is a biocompatible approach with minimal disruption to the protein substrates: 2) it circumvents the need to prepare synthetic glycans, which can be cumbersome: 3) tight control of glycoforms is achievable with the use of different enzyme combinations and: 4) the procedures are user-friendly and can be readily automated to greatly increase efficiency for industrial applications. Moreover, the idea of executing sequential enzymatic remodeling on immobilized proteins can potentially be extended to most existing biocatalytic cascade reactions involving different classes of enzymes and substrates (Huffman et al., Science 366, 1255-1259, doi: 10.1126/science.aay8484 (2019)).

A particularly disruptive application of SPGR of the present invention would be to humanize glycoforms on therapeutic proteins produced from non-human cell lines. Chinese hamster ovary (CHO) cells are commonly used for therapeutic protein production because they generate human-like post-translational modifications (Walsh, Nature Biotechnology 28, 917-924, doi: 10.1038/nbt0910-917 (2010)). However, non-human glycoforms still exist in the cell line and to reduce the potential immune response in patients, these non-human glycoforms should be removed (Liu et al. PLOS One 12, e0170013-e0170013, doi: 10.1371/journal.pone.0170013 (2017)). SPGR can be employed to humanize those glycans during the production process. From a different perspective, protein production in mammalian hosts is costly because of the long fermentation time and liability of virus infections. To address this issue, yeast has been employed as an alternative host for large-scale expression of therapeutic proteins. Glycoproteins expressed from yeast contain high-mannose N-glycans which confer a short half-life in vivo and thereby compromise the efficacy of most therapeutic proteins (Goetze et al., Glycobiology 21, 949-959, doi: 10.1093/glycob/cwr027 (2011)). Therefore, gene engineered strains are constructed for producing human-mimicking glycan patterns (Wildt & Gerngross, Nat Rev Microbiol 3, 119-128, doi: 10.1038/nrmicro1087 (2005); Laukens et al., Future Microbiol 10, 21-34, doi: 10.2217/fmb.14.104 (2015)). To date, several glycoforms have been achieved in yeast and they have provided the desired scaffold (e.g., M3 or M5 glycan and mixtures of complex N-glycans) for downstream glycan remodeling in vitro (Liu et al. Proc Natl Acad Sci USA 115, 720-725, doi: 10.1073/pnas. 1718172115 (2018)). With the combination of SPGR of the present invention, large-scale production of therapeutic proteins in yeast with controlled glycan structures is possible.

The awareness of public health has been raised significantly in the past months, intensifying the demand for developing better biologic medicines. Glycoproteins are established foundational entities in biologic medicine and vaccine development, which stresses the urgent need for exquisite control of glycosylation profiles for improved safety and efficacy. The present invention presents an efficient, user-friendly method for glycoengineering. It enables the control of glycan structures with various glycoforms and, presumably, on diverse glycoproteins.

The SPGR system of the present invention enables efficient reaction swapping, substrate purification, and the recovery of both products and engineering enzymes (Palomo, RSC Advances 4, 32658-32672, doi: 10.1039/C4RA02458C (2014); Coin et al., Nature Protocols 2, 3247-3256, doi: 10.1038/nprot.2007.454 (2007)). The present invention is particularly useful for glycoengineering antibodies. We quantitatively examined more than 30 glycan engineering enzymes for their activities on intact IgG immobilized on resins and then applied them in SPGR. This method has allowed us to harmonize IgG glycans into ten different glycoforms, including non-canonical structures, in 48 hours with an averaged conversion ratio greater than 95%. Physical and biochemical analyses indicated that the SPGR-engineered IgGs preserved integrity and functionality, suggesting that SPGR has high biocompatibility to the substrates.

In general, the SPGR system of the present invention provides a means for remodeling the N-glycans of a composition of glycoproteins comprising a heterogenous mixture or population of glycoforms, including non-human glycoforms, into a relatively homogenous mixture of glycoforms or human glycoforms as follows.

A composition of glycoproteins comprising N-glycans having a plurality of glycoforms is applied to a solid support under conditions in which the glycoproteins are reversibly immobilized on the solid support. The immobilized glycoproteins are then (i) reacted with a solution comprising a glycosylation enzyme to add or remove a sugar residue from particular or all N-glycans in the population, (ii) reacted successively with solutions of particular glycosylation enzymes to remove one or more sugar residues from particular N-glycans or all N-glycans, (iii) reacted successively with solutions comprising particular glycosylation enzymes to remove one or more sugar residues from particular N-glycans or all N-glycans and then reacted successively with solutions comprising one or more glycosylation enzymes to add one or more sugar residues to particular N-glycans or N-glycans. In embodiments where there are multiple successive removal and/or addition of sugar residues steps, between each reaction step there may be a buffer exchange step in which the reaction solution for the previous reaction step is removed and replaced with a reaction buffer for the subsequent step. Following the final reaction step, the reaction solution is removed and the immobilized glycoproteins are eluted from the solid support under conditions that disrupt the binding of the glycoprotein to the solid support to provide a composition comprising glycoproteins having remodeled N-glycans.

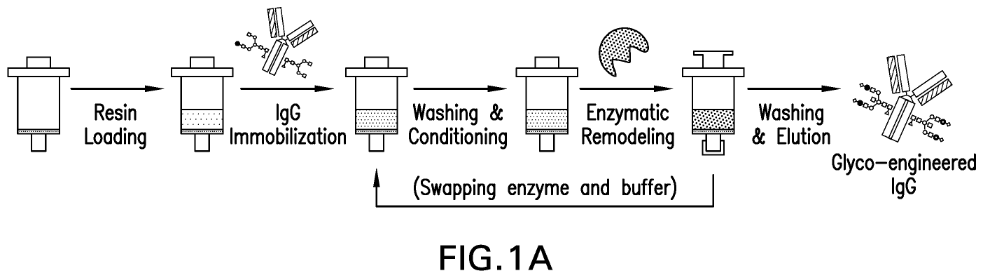

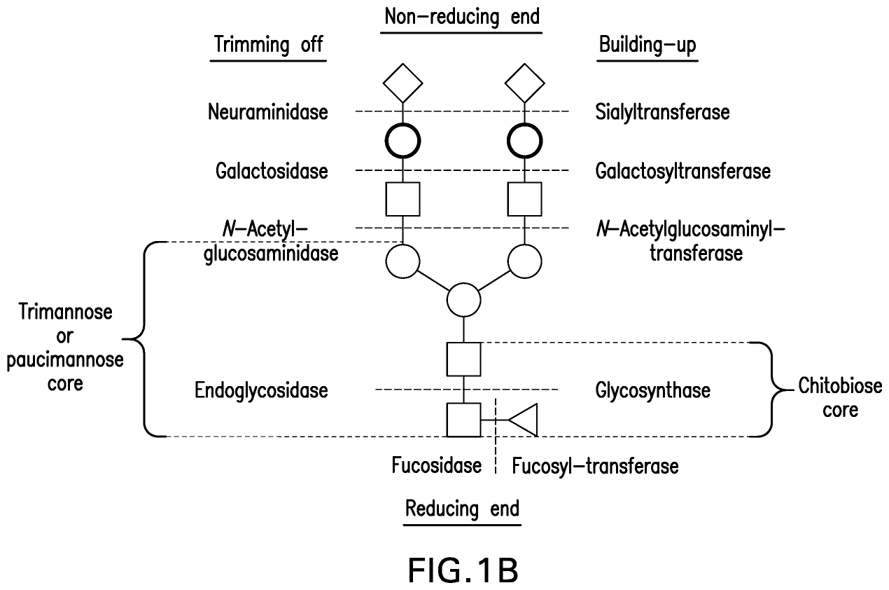

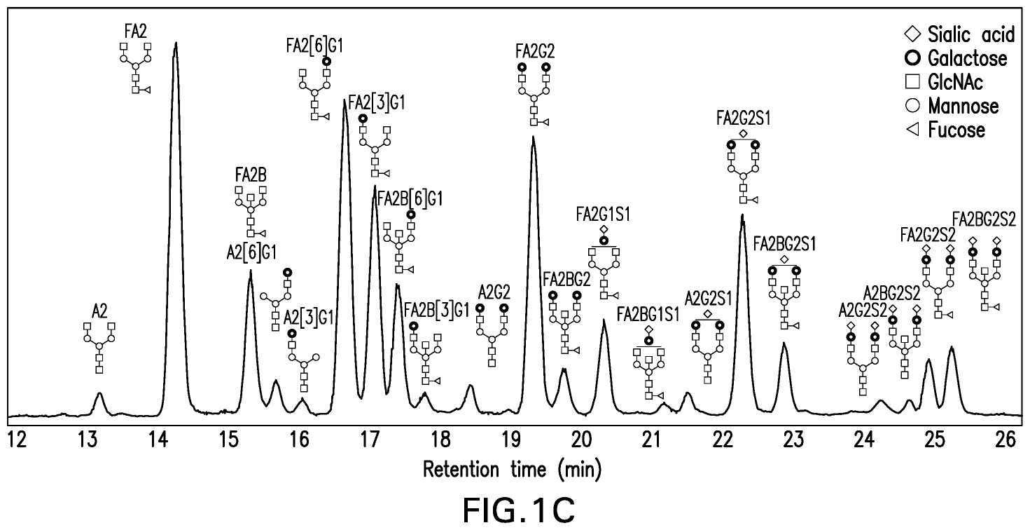

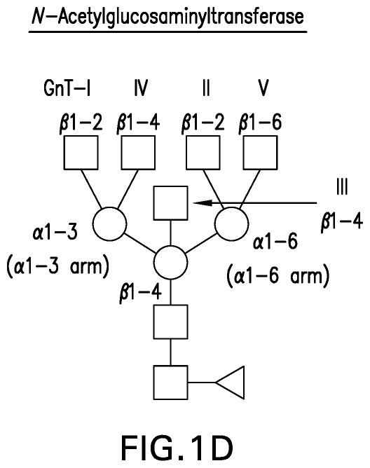

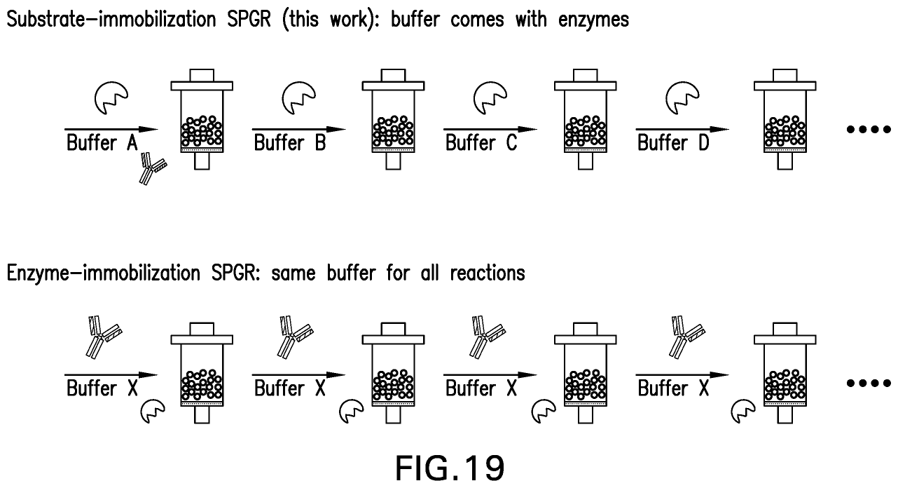

FIG. 1A and the top panel of FIG. 19 illustrate how the present invention may be used for remodeling the N-glycans of an antibody (immunoglobulin). As shown, a liquid composition of antibodies is immobilized on a solid support: the immobilized antibodies are then washed in a wash solution to remove contaminants: the washed immobilized antibodies are contacted with a solution comprising a glycosylation enzyme for a time sufficient to remodel the N-glycans on the antibodies: the antibodies comprising the remodeled N-glycans may be washed with the wash solution and eluted from the solid support or in a subsequent step contacted with a different glycosylation enzyme and washed to remodel the N-glycans on the antibodies, which may then be washed and eluted or the subsequent step repeated as many times as desired, each time with a glycosylation enzyme different from any of the previously used glycosylation enzymes. The lower panel of FIG. 19 illustrates an alternative SPGR process wherein the glycosylation enzymes are immobilized on the solid supports and the liquid composition of antibodies is applied sequentially to the immobilized glycosylation enzymes to provide a composition of antibodies or glycoproteins that have remodeled N-glycans. While the figures and Examples illustrate operation of the SPGR process with antibodies, the process may be used to remodel N-glycans of any glycoprotein. In the following embodiments. (i) the glycoproteins or antibodies may be immobilized on a solid support and the immobilized glycoproteins or antibodies are reacted sequentially with one or more glycosylation enzyme solutions, each glycosylation enzyme solution comprising a different glycosylation enzyme to produce the glycoprotein or antibodies with remodeled N-glycans or (ii) each of the glycosylation enzymes is separately immobilized on a solid support to provide a plurality of solid supports, each solid support comprising a different glycosylation enzyme, and the liquid composition of glycoproteins or antibodies is sequentially applied to two or more solid supports comprising immobilized glycosylation enzymes to produce the glycoprotein or antibodies with remodeled N-glycans.

In particular embodiments, the glycoproteins are sequentially reacted with one or more glycosidases, to remove one or more terminal sugars on the N-glycan. For example, in a first step, the glycoproteins are reacted with a first glycosidase specific for a first sugar residue at the terminus of an N-glycan. The first glycosidase removes the particular sugar residue from the termini of those N-glycans that comprise said terminal sugar residue to expose a second sugar residue at the termini of those N-glycans to provide a population of N-glycans comprising terminal second sugar residues. In a second step, the glycoproteins may then be reacted with a second glycosidase that is specific for the second sugar residue at the terminus of an N-glycan. The second glycosidase removes the second sugar residue at the termini of those N-glycans reacted with the first glycosidase and any other N-glycans that comprise the second sugar residue at the terminus to expose a third sugar residue at the termini of the N-glycans to provide a population of N-glycans comprising terminal third sugar residues. In a third step, the glycoproteins may then be reacted with a third glycosidase that is specific for third sugar residue at the terminus of an N-glycan. The third glycosidase removes the third sugar residue at the termini of those N-glycans reacted with the second glycosidase and any other N-glycans that comprise the third sugar residue at the terminus to expose a fourth sugar residue at the termini of the N-glycans to provide a population of N-glycans comprising terminal fourth sugar residues. For glycoproteins produced in mammalian or human cells, the glycoproteins may be reacted with a fucosidase which removes the sugar residue fucose from N-glycans.

In a further embodiment, following any one of the above steps, the population of N-glycans may be reacted with a glycosyltransferase capable of adding a specific sugar to the terminal sugar residues of a particular population of N-glycans. Successive reactions, each utilizing a specific glycosyltransferase, may be used to add successive sugars to the termini of the N-glycans to produce a population of N-glycans having particular glycoforms. In particular embodiments, the glycoforms comprising the population of N-glycans are homogenous or comprise a mixture of glycoforms wherein one or more glycoforms are predominant.

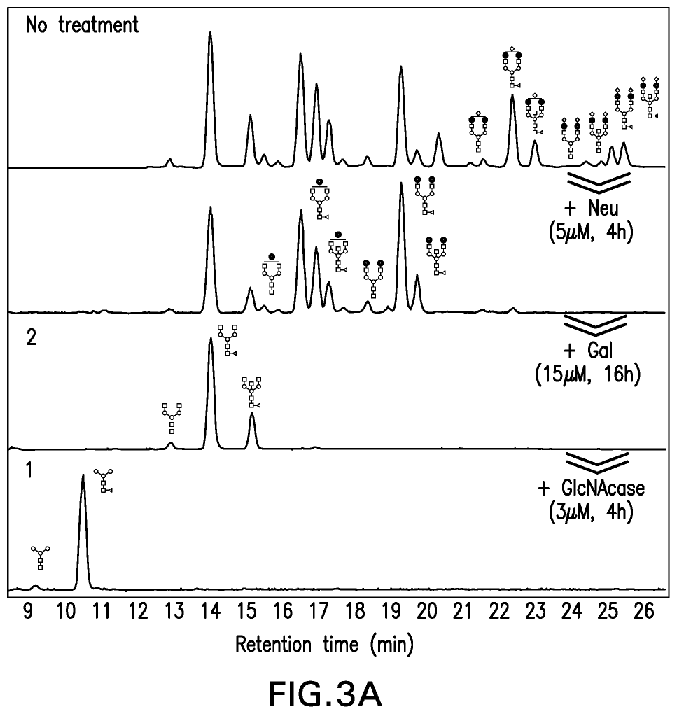

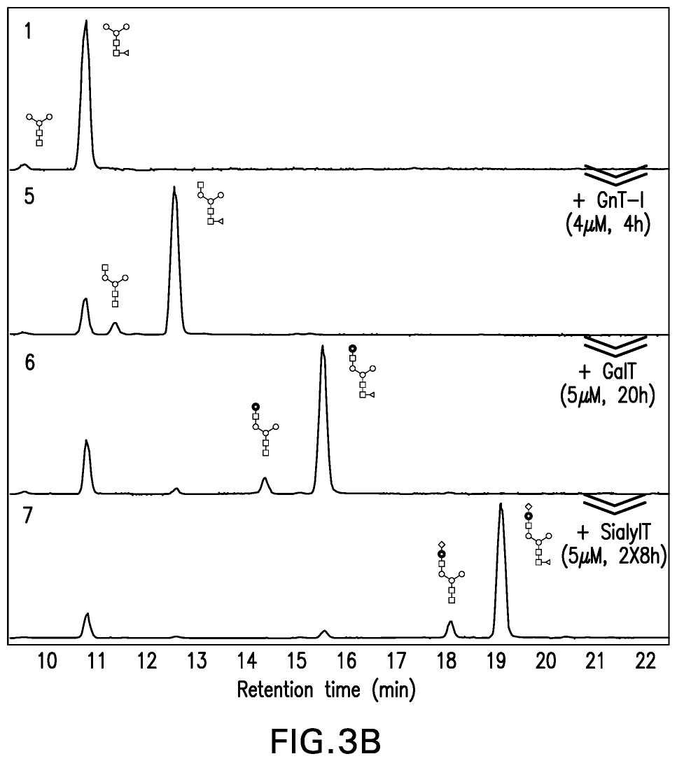

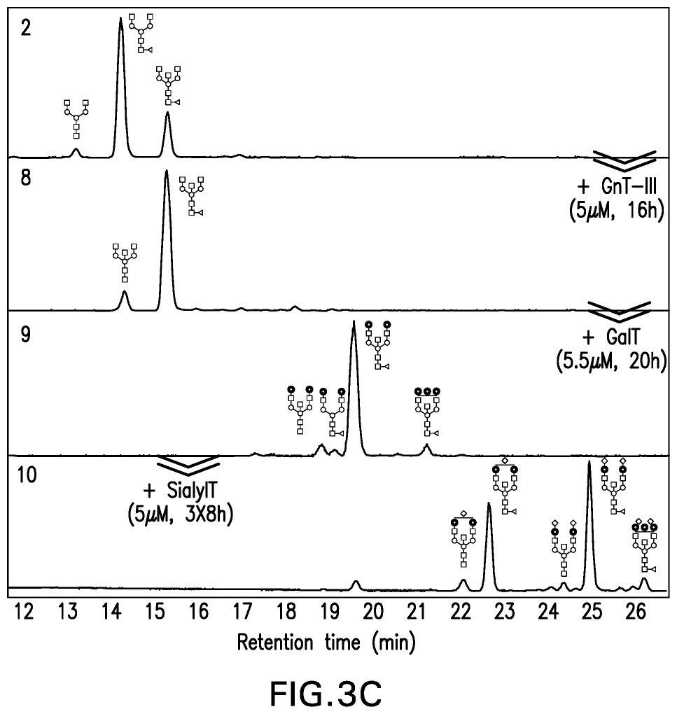

Glycoproteins produced in mammalian cells have complex N-glycans, which depending on the cell-type and the glycoprotein can have a vast array of glycoforms in differing amounts, including fucosylated and non-fucosylated bi-antennary, bisected, tri-antennary, and multi-antennary glycoforms comprising terminal sialic acid, galactose, or GlcNAc residues, or combinations thereof (for example, FIG. 1C, which shows a heterogenous population of N-glycans comprising 21 glycoforms obtained from serum IgG). The SPGR system of the present invention enables, as shown by the example in FIGS. 3A-3C, the remodeling of the complex array of 21 glycoforms into two glycoforms (FIGS. 3A and 3B) or five glycoforms (FIG. 3C). Further treatment with a fucosidase may reduce the two glycoforms in each of FIGS. 3A and 3B to one glycoform and the five glycoforms in FIG. 3C to three glycoforms.

Mammalian and human complex N-glycans comprise GlcNAc, mannose, galactose, and sialic acid residues. Complex N-glycans comprise a paucimannose core represented by the formula Man₃GlcNAc₂ forming a Y-shaped molecule wherein the terminal mannose residues are on the distal end of the chitobiose core and the GlcNAc residue at the reducing end of the chitobiose core is linked to asparagine (Asn) in a β1-N linkage. The terminal mannose residues are linked by α1-6 and α1-3 linkages to a central mannose residue to provide an α1-6-linked mannose arm and an α1-3-linked mannose arm. The central mannose is in turn linked to the chitobiose core by a β1-4 linkage. Terminal GlcNAc residues, when present, are linked to the terminal mannose residue on the α1-3-linked mannose arm in a β1-2 linkage by GnTI to produce hybrid N-glycans, and then to the terminal mannose residue on the α1-6 linked arm by GnTII to produce bi-antennary N-glycans. An additional GlcNAc residue, when present, may be linked to the terminal mannose residue on the α1-3-linked mannose arm of bi-antennary N-glycans in a β1-4 linkage by GnTIV or the terminal mannose residue on the α1-6-linked mannose arm of bi-antennary N-glycans in a β1-6 linkage by GnTV to produce tri-antennary N-glycans. In a further aspect, a GlcNAc residue, when present, may be linked to the arm of the tri-antennary N-glycan that is linked to only one GlcNAc residue with the appropriate GnT to produce multi-antennary N-glycans in which each arm comprises two GlcNAc residues, the α1-3-linked mannose arm comprising GlcNAc residues linked in a β1-2 linkage and a β1-4 linkage and the α1-6-linked mannose arm comprising GlcNAc residues linked in a β1-2 linkage and a β1-6 linkage. In an alternate aspect, the central mannose of bi-antennary N-glycans may be linked to a GlcNAc residue by a β1-4 linkage by GnTIII to produce bisected N-glycans. Terminal galactose residues, when present, are linked to the terminal GlcNAc residues of bi-antennary, tri-antennary, or multi-antennary N-glycans in a β1-4 linkage by GalT or to the α1-6-linked mannose arm and an α1-3-linked mannose arm GlcNAc residues of bisected N-glycans in a β1-4 linkage by GalT. Terminal sialic acid residues, when present, are linked to one or more of the terminal galactose residues of any of the bi-antennary, tri-antennary, multi-antennary, or bisected N-glycans comprising terminal galactose residues in either an α2-3 or α2-6 linkage by α2,3-siaT or α2,6-siaT, respectively, to produce α2,3- or α2,6-sialylated N-glycans. In particular embodiments, the sialic acid is N-Acetylneuraminic acid (Neu5Ac or NANA), a predominant sialic acid found in human cells and many mammalian cells.

Thus, in particular embodiments, the N-glycosylated glycoproteins are sequentially reacted with one or more glycosidases selected from group consisting of neuraminidase, galactosidase, and N-acetylglucosaminidase, to remove one or more terminal sugar residues on the N-glycan to produce a core structure. For example, in a first step, the glycoproteins are reacted with one or more neuraminidases to remove the sialic acid residues from those N-glycans that comprise terminal sialic acid residues to provide a population of N-glycans comprising terminal galactose residues. In a second step, the glycoproteins may then be reacted with one or more galactosidases that remove the galactose residues from those N-glycans that comprise terminal galactose residues to provide a population of N-glycans comprising terminal GlcNAc residues. In a third step, the glycoproteins may then be reacted with one or more N-acetylglucosaminidases that remove the GlcNAc residues from those N-glycans that comprise terminal galactose residues to provide a population of paucimannose N-glycans (Man₃GlcNAc₂), each N-glycan comprising a central mannose residue linked to a chitobiose core in a β1-4 linkage and two terminal mannose residues, one linked to the central mannose in an α1,3 linkage and the other linked to the central mannose in an α1,6 linkage. After any one of the three steps, the glycoproteins may be further reacted with a fucosidase to remove fucose residues from those the N-glycans comprising a fucose linked to the GlcNAc residue at the reducing end of the chitobiose core in an α1,6 linkage.

FIG. 3A illustrates the process for producing a population of glycoproteins comprising Man₃GlcNAc₂ and Man₃GlcNAc₂(Fuc) N-glycans starting from a population of glycoproteins comprising a heterogenous mixture of complex N-glycans.

In a further embodiment, following any one of the above three steps, the population of N-glycans may be reacted with a glycosyltransferase capable of adding a specific sugar to the terminal sugar residues of a particular population of N-glycans. Successive reactions, each utilizing a specific glycosyltransferase, may be used to add sugars to the termini of the N-glycans in a stepwise manner to produce a population of N-glycans having particular glycoforms. The particular glycosyltransferase that may be used in a particular reaction will depend on the N-glycan substrate to be reacted upon by the glycosyltransferase as each glycosyltransferase is specific for a particular substrate.

Thus, in one embodiment of the present invention, a population of glycoproteins comprising Man₃GlcNAc₂ N-glycans may be reacted with an GnTI in the presence of UDP-GlcNAc to produce a population of glycoproteins comprising hybrid GlcNAcMan₃GlcNAc₂ N-glycans in which the α1,3-linked mannose residue is linked to a GlcNAc residue by a β1-2 linkage. The population of glycoproteins comprising hybrid GlcNAcMan₃GlcNAc₂ N-glycans may then be reacted with a GalT in the presence of UDP-galactose to produce a population of glycoproteins comprising hybrid GalGlcNAcMan₃GlcNAc₂ N-glycans in which galactose is linked to the GlcNAc residue in a β1-4 linkage. The population of glycoproteins comprising hybrid GalGlcNAcMan₃GlcNAc₂ N-glycans may then be reacted with a α2,6- or α2,3-siaT in the presence of CMP-sialic acid to produce a population of glycoproteins comprising hybrid SiaGalGlcNAcMan₃GlcNAc₂ N-glycans in which sialic acid is linked to the galactose residue in an α2-6 or α2-3 linkage, respectively.

FIG. 3B illustrates the process for rebuilding the Man₃GlcNAc₂ and Man₃GlcNAc₂(Fuc) N-glycans of a population of glycoproteins into hybrid N-glycans. The Man₃GlcNAc₂ and Man₃GlcNAc₂(Fuc) N-glycans were reacted with a GnTI in the presence of UDP-GlcNAc to produce a population of glycoproteins comprising GlcNAcMan₃GlcNAc₂ and GlcNAcMan₃GlcNAc₂(Fuc) N-glycans. The resulting GlcNAcMan₃GlcNAc₂ and GlcNAcMan₃GlcNAc₂(Fuc) N-glycans were reacted with GalT to produce a population of glycoproteins comprising GalGlcNAcMan₃GlcNAc₂ and GalGlcNAcMan₃GlcNAc₂(Fuc) N-glycans. The resulting GalGlcNAcMan₃GlcNAc₂ and GalGlcNAcMan₃GlcNAc₂(Fuc) N-glycans were reacted with α2,6-siaT to produce a population of glycoproteins comprising SiaGalGlcNAcMan₃GlcNAc₂ and SiaGalGlcNAcMan₃GlcNAc₂(Fuc) N-glycans.

In another embodiment of the present invention, a population of glycoproteins comprising Man₃GlcNAc₂ N-glycans may be reacted with an GnTI in the presence of UDP-GlcNAc to produce a population of glycoproteins comprising hybrid GlcNAcMan₃GlcNAc₂ N-glycans in which the α1,3-linked mannose residue is linked to a GlcNAc residue by a β1-2 linkage. The population of glycoproteins comprising hybrid GlcNAcMan₃GlcNAc₂ N-glycans may then be reacted with an GnTII in the presence of UDP-GlcNAc to produce a population of glycoproteins comprising bi-antennary GlcNAc₂Man₃GlcNAc₂ N-glycans in which the α1,6-linked mannose residue is linked to a GlcNAc residue by a β1-2 linkage. The population of glycoproteins comprising bi-antennary GlcNAc₂Man₃GlcNAc₂ may then be reacted with a GalT in the presence of UDP-galactose to produce a population of glycoproteins comprising bi-antennary Gal₁₋₂GlcNAc₂Man₃GlcNAc₂ N-glycans in which galactose is linked to one or both GlcNAc residues in a β1-4 linkage. The population of glycoproteins comprising bi-antennary Gal₁₋₂GlcNAc₂Man₃GlcNAc₂ may then be reacted with a α2,6- or α2,3-siaT in the presence of CMP-sialic acid to produce a population of glycoproteins comprising bi-antennary Sia₁-2Gal₁₋₂GlcNAc₂Man₃GlcNAc₂ N-glycans in which sialic acid is linked to one or both galactose residues of bi-antennary Gal₁₋₂GlcNAc₂Man₃GlcNAc₂ in an α2-6 or α2-3 linkage, respectively.

In another embodiment of the present invention, a population of glycoproteins comprising bi-antennary GlcNac₂Man₃GlcNAc₂ N-glycans may be reacted with an GnTIII in the presence of UDP-GlcNAc to produce a population of glycoproteins comprising bisected GlcNAc₃Man₃GlcNAc₂ N-glycans in which the central mannose residue is linked to a GlcNAc residue by a β1-4 linkage. The population of glycoproteins comprising bisected GlcNAc₃Man₃GlcNAc₂ N-glycans may then be reacted with a GalT in the presence of UDP-galactose to produce a population of glycoproteins comprising bisected Gal₁₋₂GlcNAc₃Man₃GlcNAc₂ N-glycans in which galactose is linked to one or both of the terminal GlcNAc residues on the α1-3 arm or the α1-6 arm in β1-4 linkages. The population of glycoproteins comprising bisected Gal₁₋₂GlcNAc₃Man₃GlcNAc₂ N-glycans may then be reacted with a α2,6- or α2,3-siaT in the presence of CMP-sialic acid to produce a population of glycoproteins comprising bisected Sia₁-2Gal₁₋₂GlcNAc₃Man₃GlcNAc₂ N-glycans in which sialic acid is linked to one or both of the terminal galactose residues in α2-6 or α2-3 linkages, respectively.

FIG. 3C illustrates the process for remodeling bi-antennary GlcNAc₂Man₃GlcNAc₂, GlcNAc₂Man₃GlcNAc₂(Fuc), and bisected GlcNAc₃Man₃GlcNAc₂(Fuc) N-glycans of a population of glycoproteins to bisected sialylated N-glycans by reacting the population of N-glycans with GnTIII to produce a population of glycoproteins comprising bisected GlcNAcMan₃GlcNAc₂ and bisected GlcNAcMan₃GlcNAc₂(Fuc) N-glycans. In subsequent steps the bisected N-glycans were reacted with GalT and α2,6-siaT to produce a population of glycoproteins comprising the bisected sialylated N-glycans.

In another embodiment of the present invention, a population of glycoproteins comprising bi-antennary GlcNac₂Man₃GlcNAc₂ N-glycans may be reacted with an GnTIV in the presence of UDP-GlcNAc to produce a population of glycoproteins comprising α1-3 arm tri-antennary GlcNAc₃Man₃GlcNAc₂ N-glycans in which the mannose residue on the α1-3 arm is further linked to a GlcNAc residue by a β1-4 linkage. The population of glycoproteins comprising the α1-3 arm tri-antennary GlcNAc₃Man₃GlcNAc₂ N-glycans may then be reacted with a GalT in the presence of UDP-galactose to produce a population of glycoproteins comprising α1-3 arm tri-antennary Gal₁₋₃GlcNAc₃Man₃GlcNAc₂ N-glycans in which galactose is linked to one, two, or three of the terminal GlcNAc residues in β1-4 linkages. The population of glycoproteins comprising α1-3 arm tri-antennary Gal₁₋₃GlcNAc₃Man₃GlcNAc₂ N-glycans may then be reacted with an α2,6- or α2,3-siaT in the presence of CMP-sialic acid to produce a population of glycoproteins comprising α1-3 arm tri-antennary Sia₁₋₃Gal₁₋₃GlcNAc₃Man₃GlcNAc₂ N-glycans in which sialic acid is linked to one, two, or three of the terminal galactose residues in α2-6 or α2-3 linkages, respectively.

In another embodiment of the present invention, a population of glycoproteins comprising bi-antennary GlcNac₂Man₃GlcNAc₂ N-glycans may be reacted with an GnTV in the presence of UDP-GlcNAc to produce a population of glycoproteins comprising α1-6 arm tri-antennary GlcNAc₃Man₃GlcNAc₂ N-glycans in which the mannose residue on the α1-6 arm is further linked to a GlcNAc residue by a β1-4 linkage. The population of glycoproteins comprising the α1-6 arm tri-antennary GlcNAc₃Man₃GlcNAc₂ N-glycans may then be reacted with a GalT in the presence of UDP-galactose to produce a population of glycoproteins comprising α1-6 arm tri-antennary Gal₁₋₃GlcNAc₃Man₃GlcNAc₂ N-glycans in which galactose is linked to one, two, or three of the terminal GlcNAc residues in β1-4 linkages. The population of glycoproteins comprising α1-6 arm tri-antennary Gal₁₋₃GlcNAc₃Man₃GlcNAc₂ N-glycans may then be reacted with a α2,6- or α2,3-siaT in the presence of CMP-sialic acid to produce a population of glycoproteins comprising α1-6 arm tri-antennary Sia₁₋₃Gal₁₋₃GlcNAc₃Man₃GlcNAc₂ N-glycans in which sialic acid is linked to one, two, or three of the terminal galactose residues in α2-6 or α2-3 linkages, respectively.

In another embodiment of the present invention, (i) a population of glycoproteins comprising α1-3 arm tri-antennary GlcNAc₃Man₃GlcNAc₂ N-glycans may be reacted with GnTV in the presence of UDP-GlcNAc to produce a population of glycoproteins comprising multi-antennary GlcNAc₄Man₃GlcNAc₂ N-glycans in which the mannose residues on the α1-6 arm is further linked to a GlcNAc residue by a β1-6 linkage or (ii) a population of glycoproteins comprising α1-6 arm tri-antennary GlcNAc₃Man₃GlcNAc₂ N-glycans may be reacted with GnTIV in the presence of UDP-GlcNAc to produce a population of glycoproteins comprising multi-antennary GlcNAc₄Man₃GlcNAc₂ N-glycans in which the mannose residues on the α1-3 arm is further linked to a GlcNAc residue by a β1-4 linkage. The population of glycoproteins comprising the multi-antennary GlcNAc₄Man₃GlcNAc₂ N-glycans may then be reacted with a GalT in the presence of UDP-galactose to produce a population of glycoproteins comprising α1-3 arm tri-antennary Gal₁₋₄GlcNAc₄Man₃GlcNAc₂ N-glycans in which galactose is linked to one or both GlcNAc residues in a β1-4 linkage. The population of glycoproteins comprising α1-3 arm tri-antennary Gal₁₋₄GlcNAc₄Man₄GlcNAc₂ N-glycans may then be reacted with a α2,6- or α2,3-siaT in the presence of CMP-sialic acid to produce a population of glycoproteins comprising α1-3 arm tri-antennary Sia₁-4Gal₁₋₄GlcNAc₄Man₃GlcNAc₂ N-glycans in which sialic acid is linked to one or both galactose residues in an α2-6 or α2-3 linkage, respectively.

In particular embodiments of the present invention, the population of glycoproteins may comprise non-complex N-glycans such as the hypermannosylated or highly mannosylated N-glycans in yeast or filamentous fungi. Non-complex N-glycans lack the GlcNAc, Sialic acid, galactose, and fucose residues that comprise the complex N-glycans of human or mammalian glycoproteins. Glycoproteins comprising non-complex N-glycans may be remodeled using the SPGR process of the present invention provided yeast cells for producing the glycoproteins are genetically modified to produce N-glycans to reduce or eliminate the production of hyper or highly mannosylated N-glycans.

Yeast such as Saccharomyces cerevisiae and Pichia pastoris produce N-glycans that are hypermannosylated or highly mannosylated. In addition, Pichia pastoris produces N-glycans that may comprise phosphorylated N-glycans. To inhibit high or hypermannosylation, the yeast cell may be selected or engineered to be depleted in α1,6-mannosyl transferase activities, which would otherwise add mannose residues onto the N-glycan on a glycoprotein. For example, in yeast such an al, 6-mannosyl transferase activity is encoded by the OCHI gene and deletion or disruption of expression of the OCHI gene (och1Δ) inhibits the production of high mannose or hypermannosylated N-glycans in yeast such as Pichia pastoris or Saccharomyces cerevisiae. (See for example, Gerngross et al. in U.S. Pat. No. 7,029,872: Contreras et al. in U.S. Pat. No. 6,803,225; and Chiba et al. in EP1211310B1 the disclosures of which are incorporated herein by reference).

To reduce or eliminate the likelihood of N-glycans with β-linked mannose residues, which are resistant to α-mannosidases, the recombinant glycoengineered Pichia pastoris host cells are genetically engineered to eliminate glycoproteins having α-mannosidase-resistant N-glycans by deleting or disrupting one or more of the β-mannosyltransferase genes (e.g., BMT1, BMT2. BMT3, and BMT4) (See, U.S. Pat. Nos. 7,465,577, 7,713,719, and Published International Application No. WO2011046855, each of which is incorporated herein by reference). The deletion or disruption of BMT2 and one or more of BMT1, BMT3, and BMT4 also reduces or eliminates detectable cross reactivity to antibodies against host cell protein.

In such cells that lack expression of the OCHI gene and one or more of the BMT genes may be used to produce glycoproteins that have non-complex N-glycans. The glycoproteins may then be immobilized on a solid support and reacted with one or more mannosidases to produce a population of glycoproteins comprising paucimannose N-glycans. The glycoproteins comprising the paucimannose N-glycans may then be further reacted as described above to make populations of N-glycans with a desired N-glycan structures.

In an embodiment of the invention, a population of glycoproteins obtained from yeast host cells that lack OCHI and/or BMT activity comprising Man₈GlcNAc₂ N-glycans having the structure

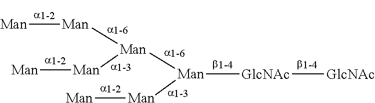

are reacted with a solution comprising one or mannosidases that remove α1-2-linked mannose residues, to produce a population of N-glycosylated glycoproteins comprising remodeled N-glycans comprising Man₅GlcNAc₂ N-glycans having the structure

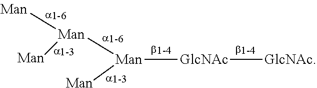

In a further embodiment, wherein the N-glycosylated glycoproteins comprising the Man₅GlcNAc₂ N-glycans are reacted with GnTI in the presence of UDP-GlcNAc to produce a population of N-glycosylated glycoproteins comprising remodeled N-glycans in which the terminal mannose residue of the α1-3 arm is further linked to a GlcNAc residue to produce hybrid GlcNAcMan₅GlcNAc₂ N-glycans having the structure

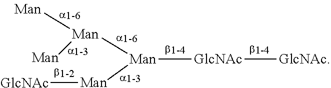

In a further embodiment, the N-glycosylated glycoproteins comprising the hybrid GlcNAcMan₅GlcNAc₂ N-glycans are reacted with a GalT in the presence of UDP-galactose to produce a population of N-glycosylated glycoproteins comprising remodeled N-glycans in which the terminal GlcNAc residue of the α1-3 arm is further linked to a galactose residue to produce hybrid GalGlcNAcMan₅GlcNAc₂ N-glycans having the structure

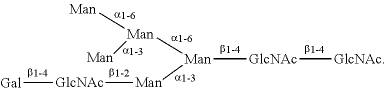

In a further embodiment, the N-glycosylated glycoproteins comprising the hybrid GalGlcNAcMan₅GlcNAc₂ N-glycans are reacted with an α2-6-SiaT in the presence of CMP-sialic acid to produce a population of N-glycosylated glycoproteins comprising remodeled N-glycans in which the terminal galactose residue of the α1-3 arm is further linked to a sialic acid residue to produce hybrid SiaGalGlcNAcMan₅GlcNAc₂ N-glycans having the structure

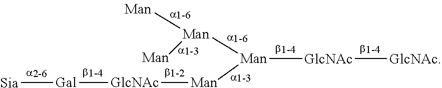

In a further embodiment, the N-glycosylated glycoproteins comprising the hybrid GalGlcNAcMan₅GlcNAc₂ N-glycans are reacted with an α2-3-SiaT in the presence of CMP-sialic acid to produce a population of N-glycosylated glycoproteins comprising remodeled N-glycans in which the terminal galactose residue of the α1-3 arm is further linked to a sialic acid residue to produce hybrid SiaGalGlcNAcMan₅GlcNAc₂ N-glycans having the structure

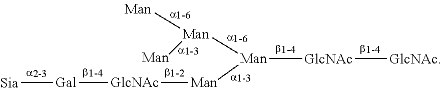

In a further embodiment, the N-glycosylated glycoproteins comprising the hybrid GlcNAcMan₅GlcNAc₂ N-glycans are reacted with a mannosidase II to produce a population of N-glycosylated glycoproteins comprising remodeled N-glycans in which the terminal mannose residues of the α1-6 arm are removed to produce hybrid GlcNAcMan₃GlcNAc₂ N-glycans having the structure

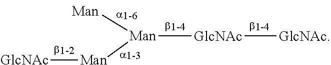

In further embodiments, the N-glycosylated glycoproteins comprising hybrid GlcNAcMan₃GlcNAc₂ N-glycans may be remodeled as disclosed supra to produce galactose-terminated or sialic acid terminated hybrid N-glycans.

In a further embodiment, the N-glycosylated glycoproteins comprising the hybrid GlcNAcMan₃GlcNAc₂ N-glycans are reacted with a GnTII in the presence of UDP-GlcNAc to produce a population of N-glycosylated glycoproteins comprising remodeled N-glycans in which the terminal mannose residue of the α1-6 arm is further linked to a GlcNAc residue to produce bi-antennary GlcNAc₂Man₃GlcNAc₂ N-glycans having the structure

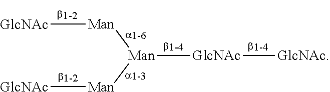

In further embodiments, the N-glycosylated glycoproteins comprising bi-antennary GlcNAc₂Man₃GlcNAc₂ N-glycans may be remodeled as disclosed supra to produce galactose-terminated and/or sialic acid terminated bi-antennary N-glycans.

In particular embodiments of the invention, the glycoforms comprising the population of remodeled N-glycans are homogenous or comprise a mixture of glycoforms wherein one glycoform is predominant. In particular embodiments, the predominant glycoform comprises at least 90% of the glycoforms in the population of remodeled N-glycans. In particular embodiments, the predominant glycoform comprises at least 80% of the glycoforms in the population of remodeled N-glycans. In particular embodiments, the predominant glycoform comprises at least 70% of the glycoforms in the population of remodeled N-glycans. In particular embodiments, the predominant glycoform comprises at least 60% of the glycoforms in the population of remodeled N-glycans. In particular embodiments, the predominant glycoform comprises at least 50% of the glycoforms in the population of remodeled N-glycans. In particular embodiments, the remodeled N-glycans comprise only one detectable glycoform.

In particular embodiments of the invention, the glycoforms comprising the population of remodeled N-glycans comprise a mixture of glycoforms wherein two or three glycoforms comprise at least 50% of the glycoforms in the population of remodeled N-glycans. In particular embodiments, at least 60% of the glycoforms of the remodeled N-glycans comprise two or three glycoforms. In particular embodiments, at least 70% of the glycoforms of the remodeled N-glycans comprise two or three glycoforms. In particular embodiments, at least 80% of the glycoforms of the remodeled N-glycans comprise two or three glycoforms. In particular embodiments, at least 90% of the glycoforms of the remodeled N-glycans comprise two or three glycoforms. In particular embodiments, the detectable glycoforms of the remodeled N-glycans comprise two or three glycoforms.

In particular embodiments, populations of glycoproteins comprising any one of the above hybrid or complex N-glycans may be reacted with an α1-6-fucosyltransferase, which transfers a fucose residue from GDP-fucose to the GlcNAc residue on the reducing end of the chitobiose core in an α1-6 linkage.

### Glycosylation Enzymes

Glycosylation enzymes include exoglycosidases, endoglycosidases, and glycosyltransferases, many of which are type-II membrane proteins. The SPGR process of the present invention may be performed using any glycosylation enzyme know in the art when used under conditions suitable for enzyme activity. Many glycosylation enzymes are commercially available whereas others are encoded by nucleotide sequences that are available in the art and can be cloned and synthesized in vitro using well-known molecular biology techniques. As used herein, a glycosylation enzyme may be a recombinant enzyme that comprises the catalytic domain of the enzyme and lacks the transmembrane and/or native signal or leader domains.

The Repository for Glyco-enzyme Expression Constructs located at the Complex Carbohydrate Research Center, 315 Riverbend Road, The University of Georgia, Athens, Georgia 30602 USA (accessible on-line at http://glycoenzymes.ccrc.uga.edu/) have reported that expression constructs encoding all mammalian glycosylation enzymes has been generated, including glycosyltransferases, glycoside hydrolases, and glycan modifying enzymes. Many of the expression constructs express the glycosylation enzyme as a secreted soluble enzymes catalytic domains (when possible), including glycosylation enzymes with truncated transmembrane domains, replaced with signal sequences and fusion tags or other larger fusion proteins to facilitate detection, quantitation, and affinity purification, and includes expression vectors for production in mammalian cells, insect cells (baculovirus), and bacteria and protocols for protein production in each host system. The expression constructs are available from the DNASU Plasmid Repository located at DNASU/PSI: Biology-MR, Center for Personalized Diagnostics, Biodesign Institute, Arizona State University, 1001 S. McAllister Ave, Tempe, AZ 85287 USA (accessible on-line at https://dnasu.org/DNASU/Home.do)

The following examples are intended to promote a further understanding of the present invention.

### Example 1

### Materials and Instruments

Human serum IgG (14506), Tris-HCl (T-5941), HEPES (H4034), Sodium acetate (S2889), Calcium chloride (C5670), Magnesium chloride (M4880), Manganese Chloride solution (M1787), Sodium chloride solution (S5150), CMP-NANA (C8271), UDP-Gal (U4500), UDP-GlcNAc (U4375), GDP-Fuc (G4401), Acetonitrile (900667), Discovery glycan solid phase extraction (SPE) columns (55465-U), and empty SPE column and frit (57607-U) are purchased from Sigma-Aldrich. Protein A resin (53139), Protein A-IgG binding buffer (54200), Protein A-IgG elution buffer (21027), H2O (10977015) were purchased from ThermoFisher. Hydrophilic interaction liquid chromatography (HILIC) columns for chromatography (186004742), SPE uPlate (186002780), uPlate extraction manifold (186001831), SPE vacuum manifold (WAT200607), RapiGest SF (186008090), Glycoworks buffer (186008100), glycan quantitative standard (186008791), and Rapifluor-MS (186008091) were purchased from Waters. Molecular weight cutoff (MWCO) filters (UFC503096) were purchased from Millipore Sigma. Rapid PNGaseF (P0710) was purchased from NEB. Human FcR AlphaLISA Binding Kit (AL3081C) was purchased from Perkin Elmer.

Chromatography and mass spectrometry analyses were conducted using an Agilent 1290 Infinity II LC system tandem with Agilent 6500 Series quadrupole time-of-flight MS system. Enzyme concentration was determined by absorbance at 280 nm using NanoDrop 2000 (Thermo Scientific). Temperature-controlled reactions/incubations were performed in ThermoFisher MaxQ 6000 incubator and Fisherbrand Thermal Mizer II. Measurements of hydrodynamic diameter, melting temperature (Tm) and aggregation temperature (Tagg) were performed on Uncle (all-in-one biologics stability screening platform) from Unchained Labs. The detection of AlphaLISA-based assays for IgG-FcγR binding studies were conducted by EnVision Multimode Plate Reader 2105 (PerkinElmer). Refer to Tables 1A-C for the vendor information of glycosylation enzymes.

TABLE 1A
Activity Screening of Glycosylation enzymes

| | | | | Conversion Ratio (%) | | |
|---|---|---|---|---|---|---|
| Enzyme | Source | Size | Target | 1 hour | 24 hours | Vendor & Cat. # |
| Neuraminidase | C. perfringens | 43 | Terminal sialic acid | 66.4 ± 4.7 | 90.2 ± 1.3 | NEB P0720 |
| Neuraminidase A | A. ureafaciens | 100 | Terminal sialic acid | 55 ± 3.5 | 56.5 ± 1.2 | NEB P0722 |
| Neuraminidase S | S. pneumoniae | 74 | Terminal sialic acid | N.D. | 9.6 ± 1.6 | NEB P0743 |
| Galactosidase S | S. pneumoniae | 231 | Terminal galactose | 5.5 ± 2.2 | 51 ± 8.1 | NEB P0745 |
| Galactosidase | B. taurus (testis) | 71 | Terminal galactose | 3.5 ± 0.9 | 3.7 ± 1.6 | NEB P0746 |
| Galactosidase 1 | H. sapiens | 74 | Terminal galactose | N.D. | 3 ± 0.7 | R&D Systems 6464-GH |
| N-Acetylglucosaminidase S | S. pneumoniae | 125 | Terminal GlcNAc | 47 ± 1 | 82.9 ± 1.8 | NEB P0744 |
| N-Acetylhexosaminidase F | S. picatus | 100 | Terminal GlcNAc | N.D. | N.D. | NEB P0721 |
| Fucosidase | B. Taurus (kidney) | 52 | Terminal Fucose | N.D. | 1.2 ± 0.9 | NEB P0748 |
| Fucosidase O | C. Omnitrophica | 49 | Terminal Fucose | 1.3 ± 0.2 | 2.4 ± 1 | NEB P0749 |
| Fucosidase | Prunus dulcis | 56 | Terminal Fucose | N.D. | N.D. | NEB P0769 |
| Fucosidase | C. meningosepticum | 50 | Terminal Fucose | N.D. | N.D. | Sigma 344826 |
| Fucosidase | E. miricola | 56 | Terminal Fucose | N.D. | N.D. | Sigma F6272 |
| Fucosidase | H. sapiens | 51 | Terminal Fucose | N.D. | N.D. | R&D Sys. 7039-GH |
| Fucosidase | T. maritima | 54 | Terminal Fucose | N.D. | N.D. | R&D Sys. 6556-GH |

Enzyme activity was determined by using SPGR protocol with 1 mg human serum IgG (66.7 μM) and glycosylation enzyme (0.25 μM) for 1 or 24 hours. HILIC-MS was employed for characterizing glycan structures and quantification.
N.D. = not detectable.
V = enzymes showed the highest activity in the screening (selected for SPGR). 0.1 ml Protein A resin was used for immobilization and the final reaction volume was 0.1 ml for all the reactions. Reactions in this screening were conducted using the working conditions suggested by the vendors without further optimization.
*Glycoengineered IgG was used as the substrate for the screening.

TABLE 1B
Activity Screening of Glycosylation enzymes

| | | | | Conversion Ratio (%) | | |
|---|---|---|---|---|---|---|
| Enzyme | Source | Size | Target | 1 hour | 24 hours | Vendor & Cat. # |
| Endoglycosidase A | A. protophormia | 69 | GlcNAc-GlcNAc linkage | N.D. | N.D. | Chemily G. EN01017 |
| Endoglycosidase D | S. pneumoniae | 140 | GlcNAc-GlcNAc linkage | N.D. | N.D. | NEB P0742 |
| Endoglycosidase F2 | E. miricola | 40 | GlcNAc-GlcNAc linkage | N.D. | N.D. | NEB P0772 |
| Endoglycosidase F3 | E. minicola | 39 | GlcNAc-GlcNAc linkage | 17.1 ± 5.7 | 56.7 ± 13.4 | NEB P0771 |
| Endoglycosidase M | M. hiemalis | 85 | GlcNAc-GlcNAc linkage | N.D. | N.D. | TCI A1651 |
| Endoglycosidase S | S. pyogenes | 136 | GlcNAc-GlcNAc linkage | 39.9 ± 1.9 | 68.4 ± 5.2 | NEB P0741 |

Enzyme activity was determined by using SPGR protocol with 1 mg human serum IgG (66.7 μM) and glycosylation enzyme (0.25 μM) for 1 or 24 hours. HILIC-MS was employed for characterizing glycan structures and quantification.
N.D. = not detectable.
V = enzymes showed the highest activity in the screening (selected for SPGR). 0.1 ml Protein A resin was used for immobilization and the final reaction volume was 0.1 ml for all the reactions. Reactions in this screening were conducted using the working conditions suggested by the vendors without further optimization.
*Glycoengineered IgG was used as the substrate for the screening.

TABLE 1C
Activity Screening of Glycosylation enzymes

| | | | | Conversion Ratio (%) | | |
|---|---|---|---|---|---|---|
| Enzyme | Source | Size | Target | 1 hour | 24 hours | Vendor & Cat. # |
| α2-6 Sialyltransferase | P. damsela | 59 | Terminal Galactose | N.D. | N.D. | Sigma S2076 |
| α2-6 Sialyltransferase | P. multocida | 46 | Terminal Galactose | N.D. | N.D. | Sigma S1951 |
| α2-6 Sialyltransferase (ST6Gal1) | H. sapiens | 44 | Terminal Galactose | 7.8 ± 1.6 | 29.3 ± 0.4 | Sigma SAE0090 |
| α2-6 Sialyltransferase (ST6Gal2) | H. sapiens | 33 | Terminal Galactose | N.D. | 1.6 ± 1.2 | R&D Systems 8330-GT |
| α2-6 Sialyltransferase (ST6GalNAc4) | H. sapiens | 31 | Terminal Galactose | N.D. | 1.2 ± 0.4 | R&D Systems 6876-GT |
| α2-3 Sialyltransferase (ST3-Gal1) | H. sapiens | 33 | Terminal Galactose | N.D. | 2.1 ± 0.4 | R&D Systems 6905-GT |
| α2-3 Sialyltransferase (ST3-Gal2) | H. sapiens | 35 | Terminal Galactose | N.D. | 1.2 ± 1.1 | R&D Systems 7275-GT |
| β1-4 Galactosyltransferase 1 | Homo sapiens | 40 | Terminal GlcNAc | 7 ± 1.8 | 22.2 ± 1.1 | Sigma SAE0093 |
| β1-4 Galactosyltransferase | B. Taurus (milk) | 45 | Terminal GlcNAc | 3.1 ± 1 | 23 ± 1.2 | Sigma G5507 |
| N-Acetylglucosaminyltransferase 1 | H. sapiens | 48 | (F)M3 glycoforms | 8.4 ± 2.6* | 17.6 ± 0.2* | R&D Systems 8334-GT |
| N-Acetylglucosaminyltransferase 3 | H. sapiens | 58 | (F)A2 glycoforms | N.D. | 6 ± 2.1 | R&D Systems 7359-GT |
| N-Acetylglucosaminyltransferase 5 | H. sapiens | 65 | (F)A2/(F)A3 glycoforms | N.D. | N.D. | R&D Systems 5469-GT |

Enzyme activity was determined by using SPGR protocol with 1 mg human serum IgG (66.7 μM) and glycosylation enzyme (0.25 μM) for 1 or 24 hours. HILIC-MS was employed for characterizing glycan structures and quantification.
N.D. = not detectable.
V = enzymes showed the highest activity in the screening (selected for SPGR). 0.1 ml Protein A resin was used for immobilization and the final reaction volume was 0.1 ml for all the reactions. Reactions in this screening were conducted using the working conditions suggested by the vendors without further optimization.
*Glycoengineered IgG was used as the substrate for the screening.

### Methods

### Solid-Phase Glycan Remodeling (SPGR, See FIG. 1A)

(I) Resin loading: Empty SPE columns composed of an empty column body, a frit with 0.2 μm pores, and a lid were used as reaction vessels for SPGR reactions. The columns were mounted onto a 20-wells SPE vacuum manifold. 100 μL of protein A resin (wet resin) was transferred into each column, followed by conditioning with 0.8 mL protein A-IgG binding buffer twice. A vacuum system was connected to the manifold to control the flow rate.

(II) IgG immobilization: 1 mg (unless other specified) human serum IgG was added to 0.5 mL protein A-IgG binding buffer, followed by gently shaking until all the powder was dissolved. The solution was then transferred to the SPE column containing protein A resins. To ensure good immobilization, the columns were dismounted from the manifold, capped with Luer fittings, and then incubated for 15 minutes at room temperature with gentle rotating.

(III) Washing and conditioning: After the incubation, the columns were mounted to the manifold again, followed by washing with 0.8 mL protein A-IgG binding buffer (3 times) and then enzyme reaction buffer (2 times). After the conditioning step, the buffer was completely drained out from the columns.

(IV) Enzyme reactions: Enzyme reaction solutions were prepared by mixing the desired amount of enzyme, reaction buffer, and saccharide donors (1 mM, for glycosyltransferase) to a final volume of 100 μL unless other specified. Refer to Table 2 and Table 3 for the details of reaction conditions, including pH, concentration, and cation cofactors.

TABLE 2
Glycosylation enzymes selected for SPGR and their optimized working conditions.

| | | | | T | Cation | CR₅₀ |
|---|---|---|---|---|---|---|
| Enzyme | Source (Gene) | Function (on IgG glycans) | pH | (° C.) | Co-factor | (%) |
| Neuraminidase (Sialidase) | C. perfringens (nanH) | Removes terminal α2,6-linked sialic acid | 5.5† | 42 | Ca²⁺† | 1.0 |
| Galactosidase S | S. pneumoniae (bgaA) | Removes terminal β1,4-linked galactose | 5.5† | 37 | Ca²⁺† | 4.7 |
| N-Acetylglucosaminidase S (GlcNAcase) | S. pneumoniae (strH) | Removes terminal β1,2-and β1,4-linked | 5.5† | 42 | Ca²⁺† | 0.4 |
| | | GlcNAc | | | | |
| Fucosidase O* | C. Omnitrophica [symbol] | Removes terminal α1,6-linked fucose | 4.5† | 50† | None† | N.D. |
| Endoglycosidase S | S. pyogenes (ndoS) | Liberates glycan moiety through β1-4 | 5.5† | 42 | Ca²⁺† | 0.7 |
| | | GlcNAc-GlcNAc bond cleavage | | | | |
| α2-6 Sialyltransferase | H. sapiens (ST6GAL1) | Adds sialic acid to terminal galactose | 7.5 | 37 | Mg2+ | 15.2 |
| | | through α1,6 linkage | | | | |
| β1-4 Galactosyltransferase 1 | H. sapiens (B4GALT1) | Adds galactose to terminal GlcNAc through | 7.0 | 50 | Mn2+ | 7.0 |
| | | β1,4 linkage | | | | |
| N-Acetylglucosaminyltransferase 1* | H. sapiens (MGAT1) | Adds GlcNAc to terminal α1,3-linked | 7.5 | 30 | Na⁺ Ca²⁺ | 3.8 |
| | | mannose through β1,2 linkage | | | Mg²⁺ Mn²⁺ | |
| N-Acetylglucosaminyltransferase 3 | H. sapiens (MGAT3) | Adds GlcNAc to terminal β1,4-linked | 6.5 | 30 | Na⁺ | 7.9 |
| | | mannose through β1,4 linkage | | | Mn²⁺ | |
| Fucosyltransferase* | H. sapiens (FUT8) | Adds fucose to the innermost GlcNAc | 7.0† | 37† | Na⁺† | N.D. |
| | | through α1,6 linkage | | | | |

CR₅₀: the enzyme-to-substrate (IgG) molar ratio required for reaching 50% substrate conversion into the products in one hour using SPGR and the optimized conditions. Please refer to Table 3 for detailed working conditions.
N.D. = no data.
†The conditions suggested by the enzyme suppliers were used without further optimization.
*Glycoengineered IgG was used as the substrate for the characterization. [symbol]
Genbank KXK31601.1

Enzyme reactions were initiated by transferring the reaction solution into SPE columns that contain immobilized IgG substrates. The columns were capped by Luer fitting, sealed with parafilm, and incubated at temperature-controlled shakers for a certain amount of time (Table 3). After the reaction, the enzyme solution was discarded (or recovered) and the 5 columns were washed 6-8 times with 0.8 mL protein A-IgG binding buffer via the extraction manifold.

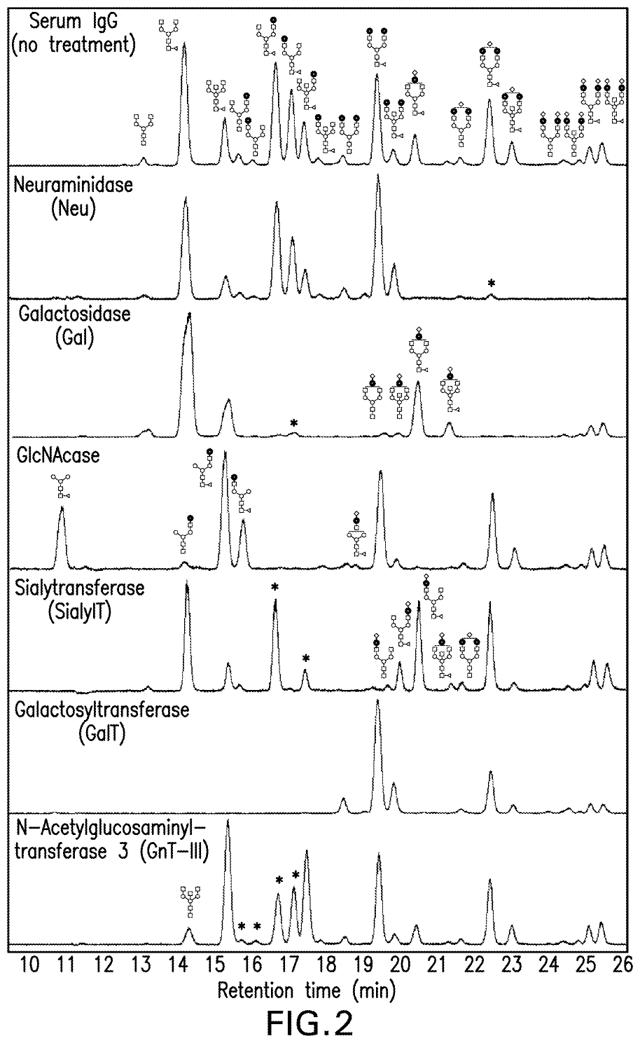

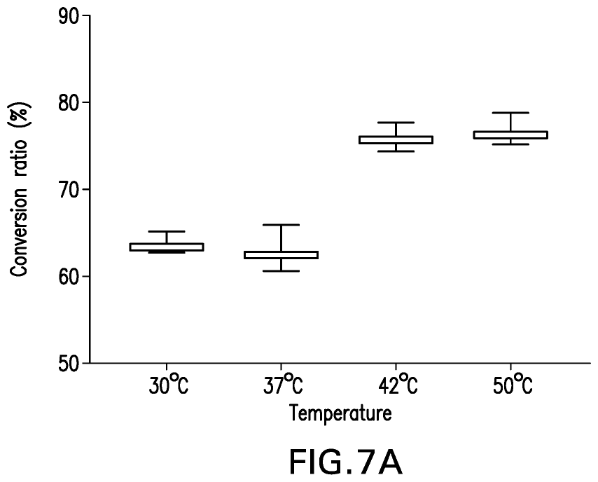

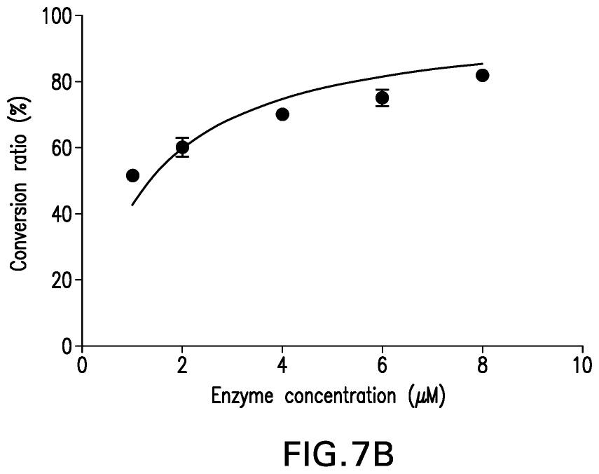

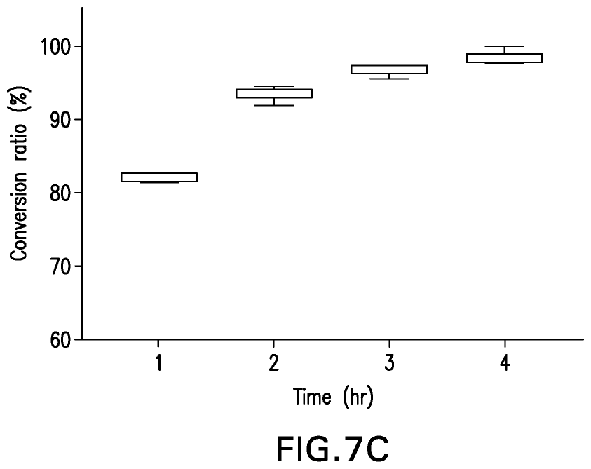

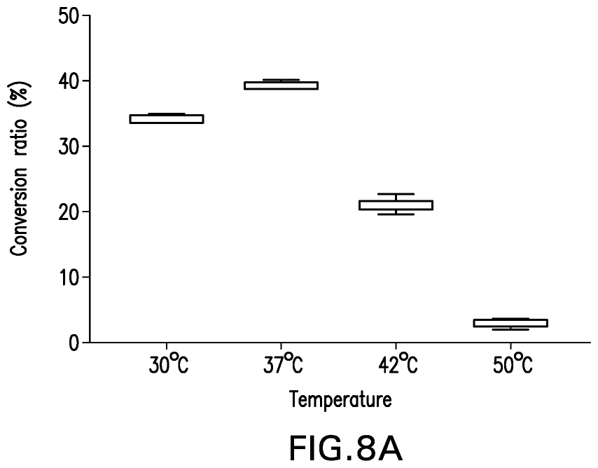

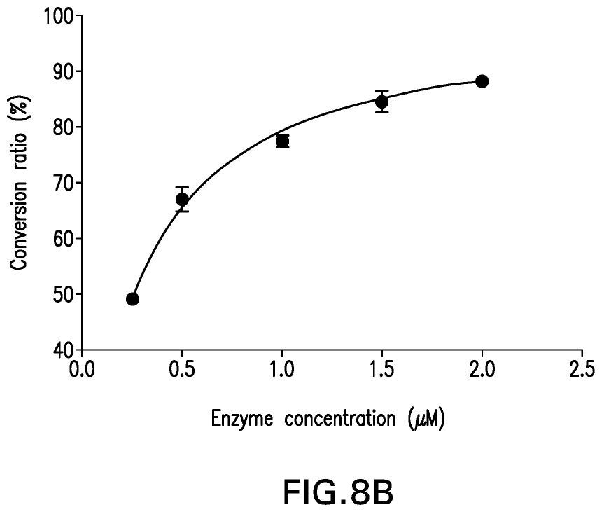

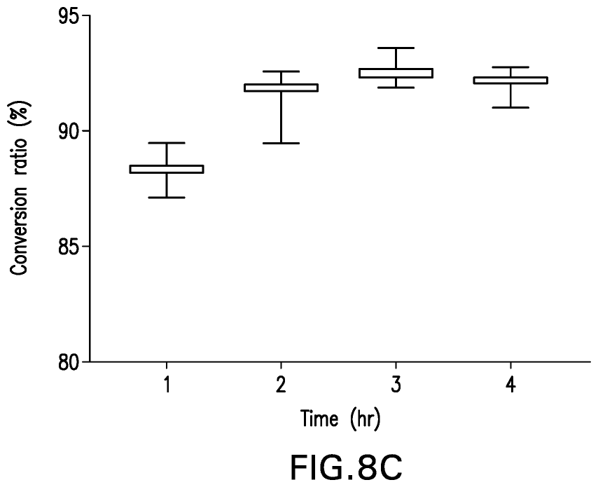

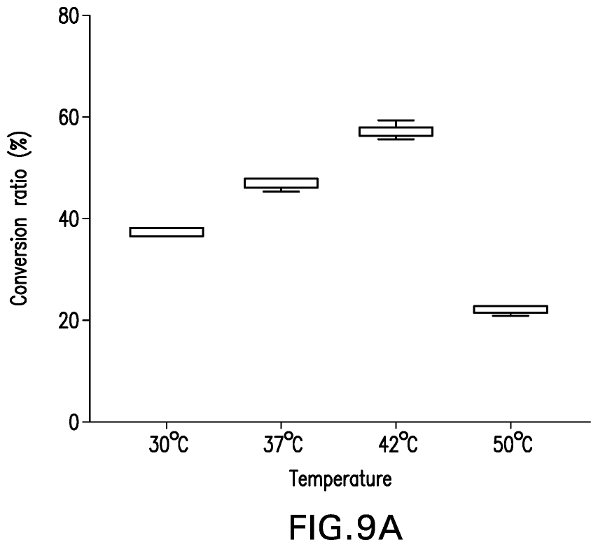

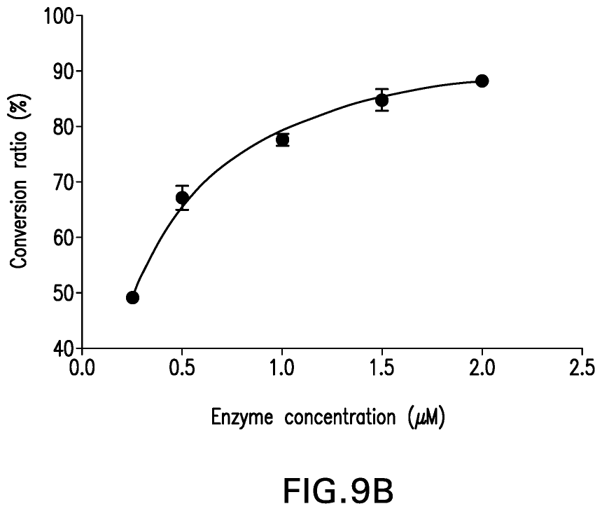

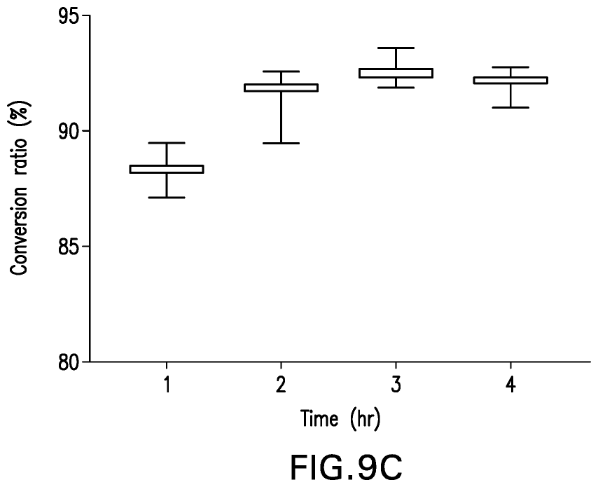

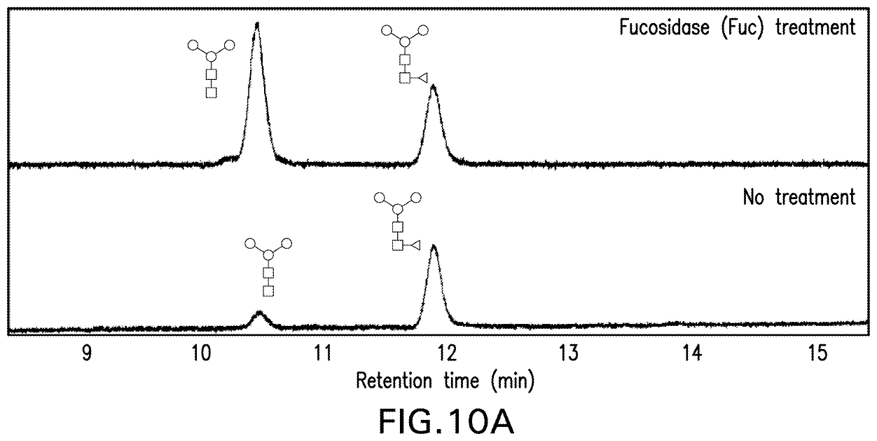

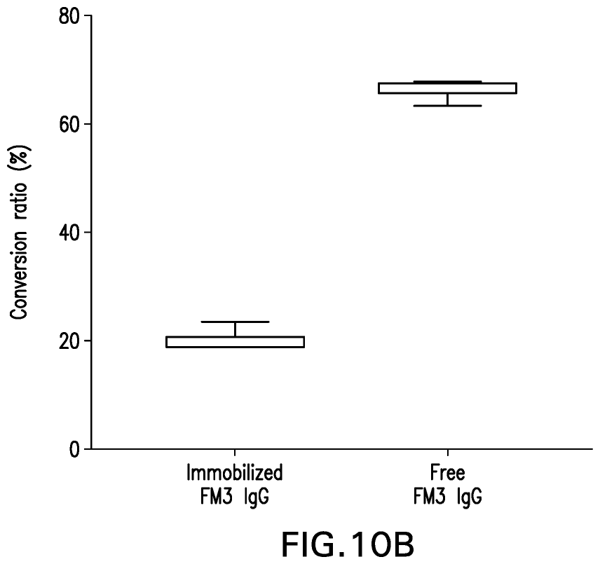

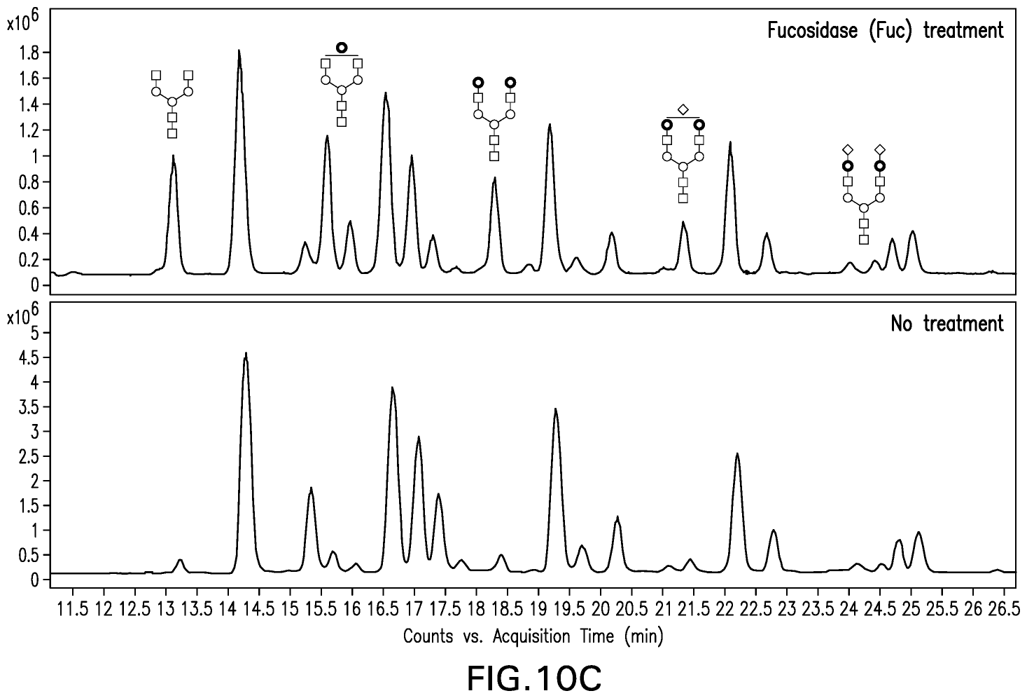

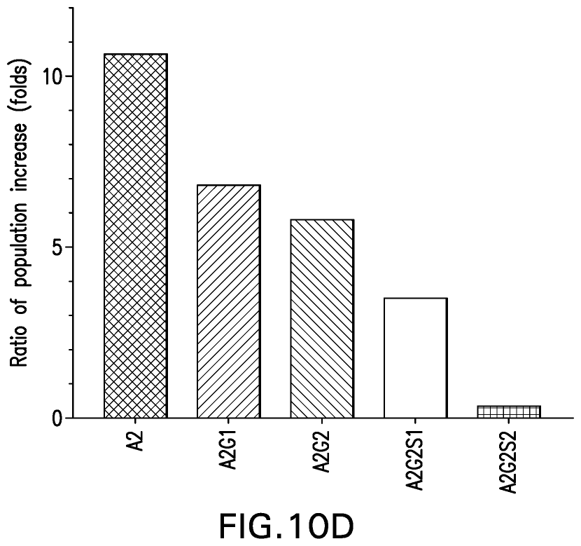

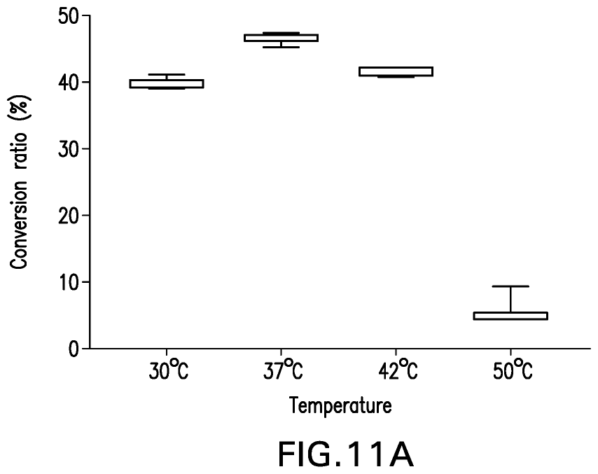

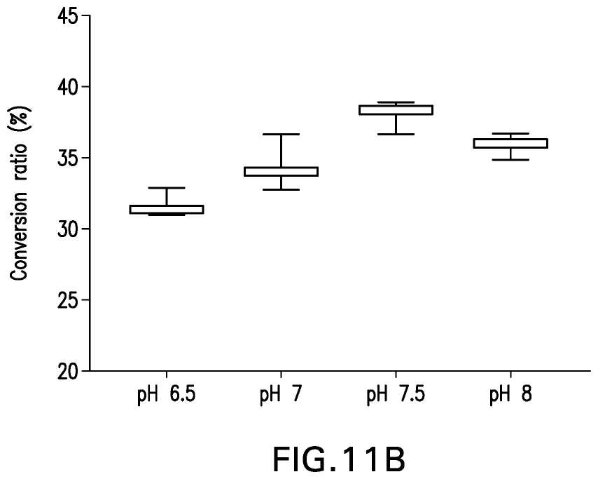

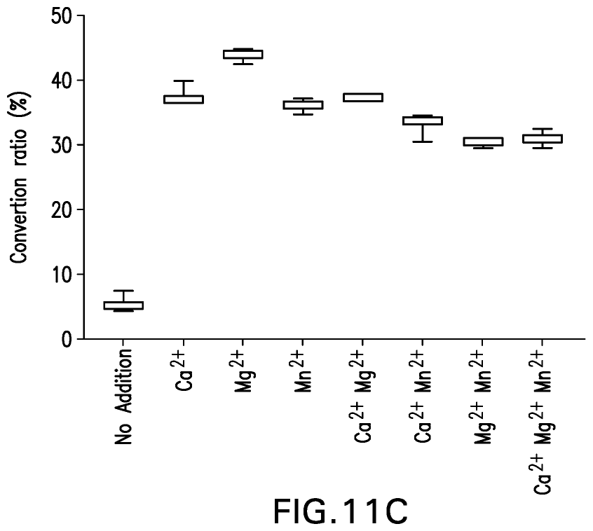

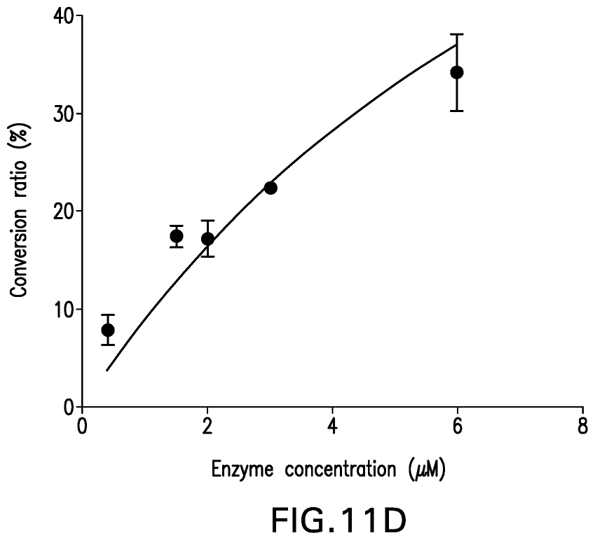

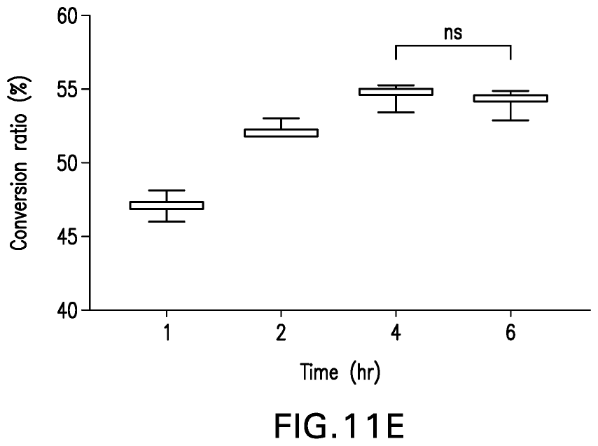

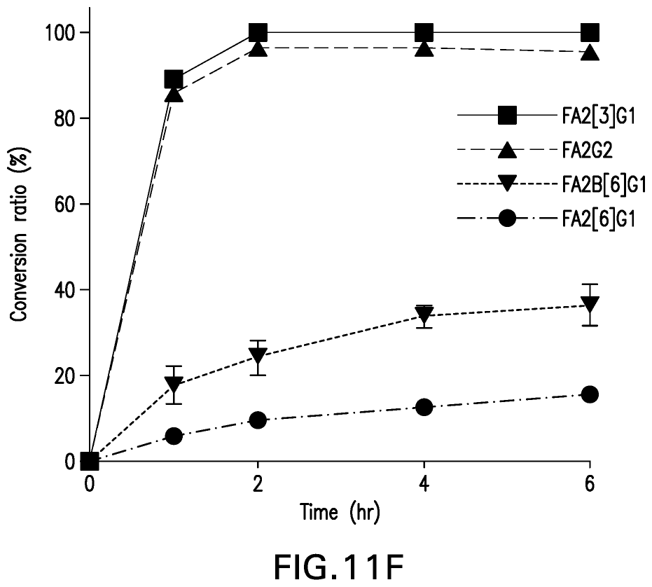

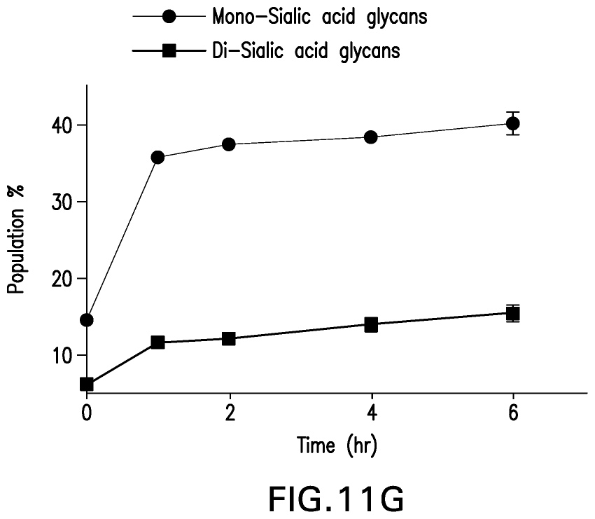

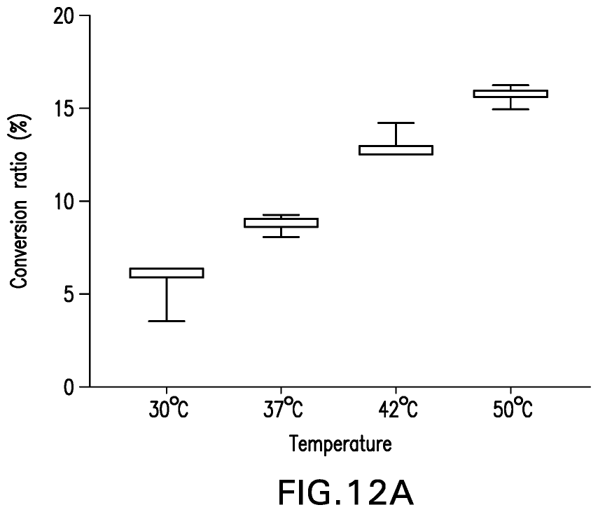

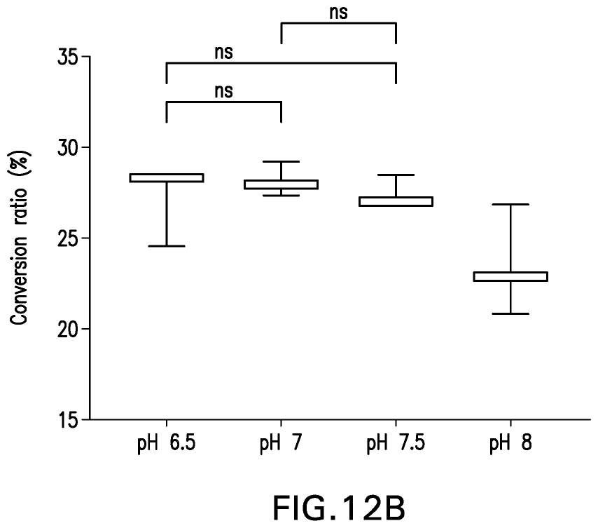

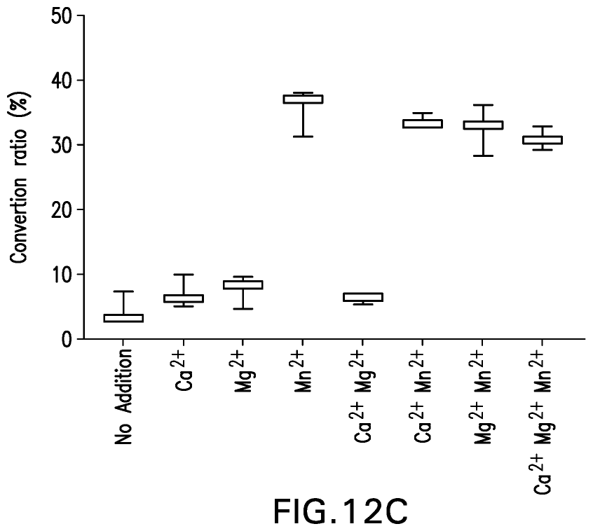

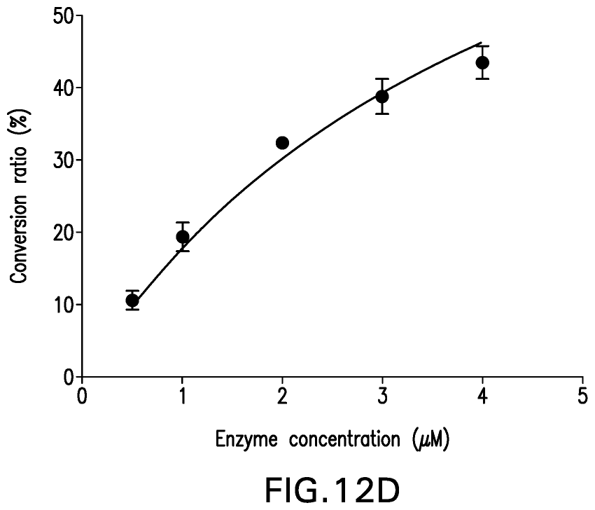

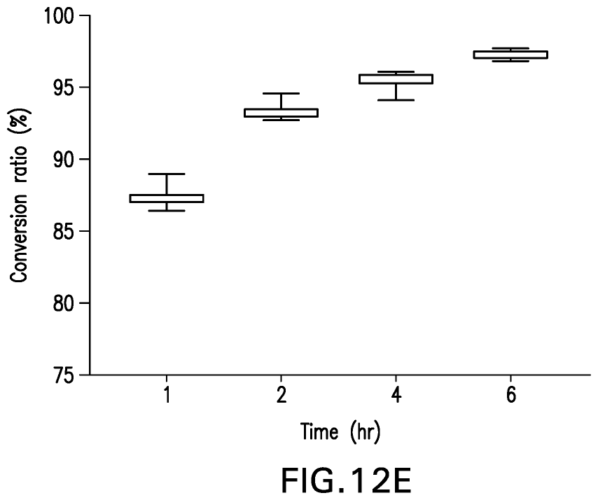

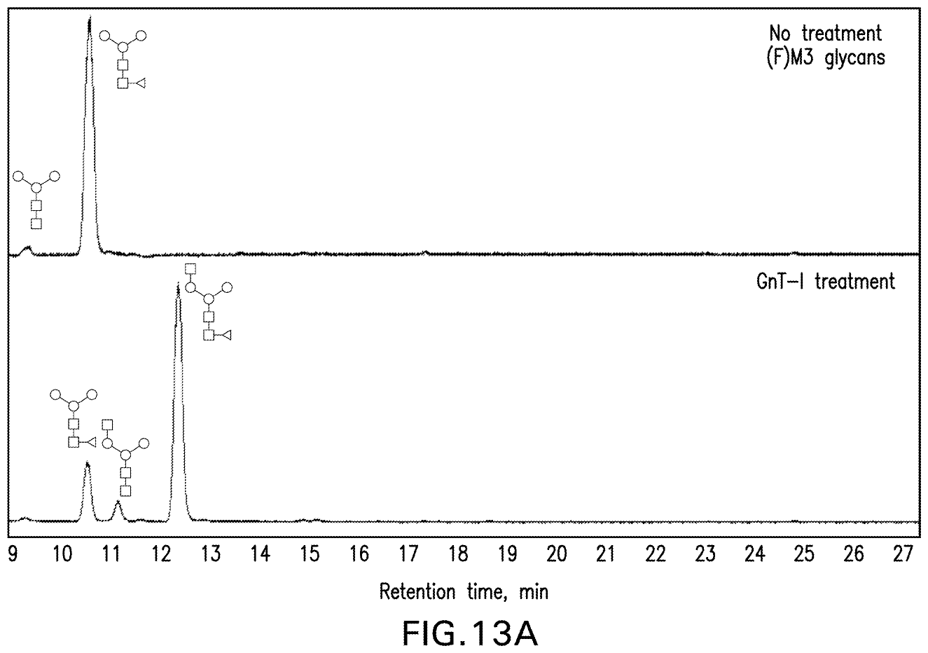

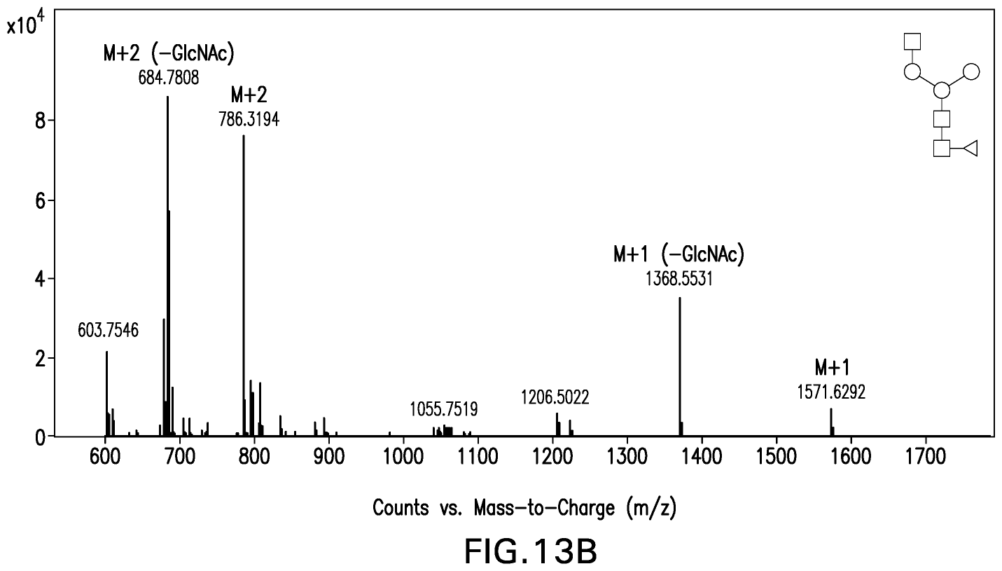

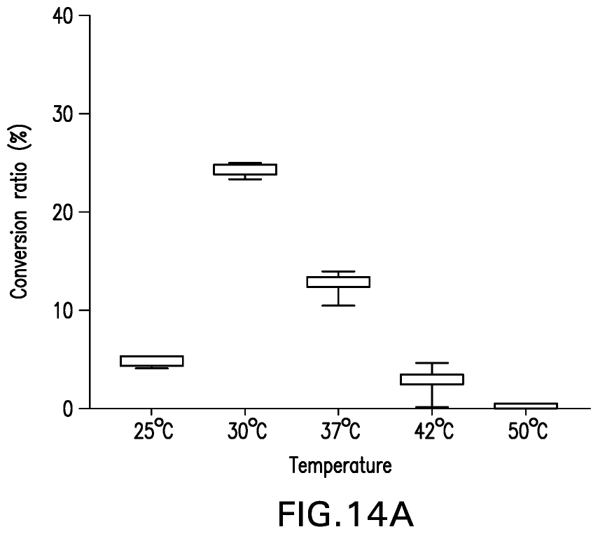

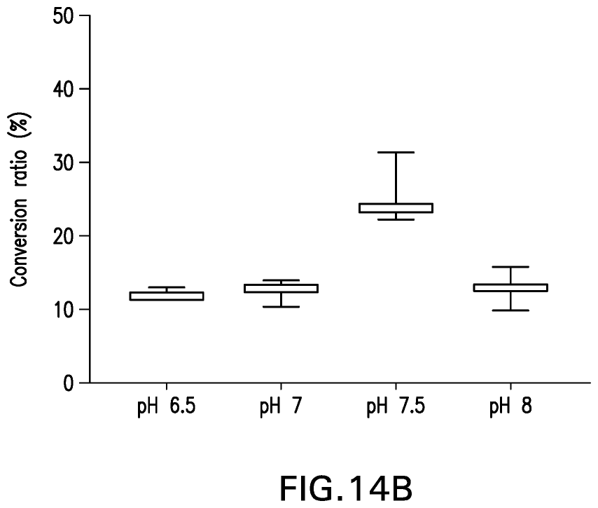

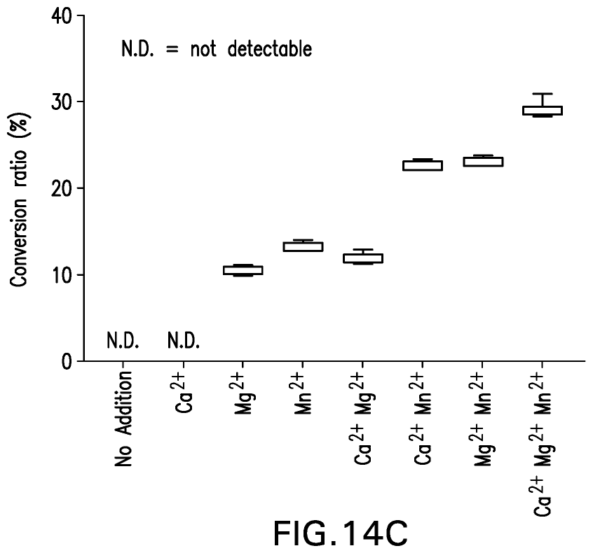

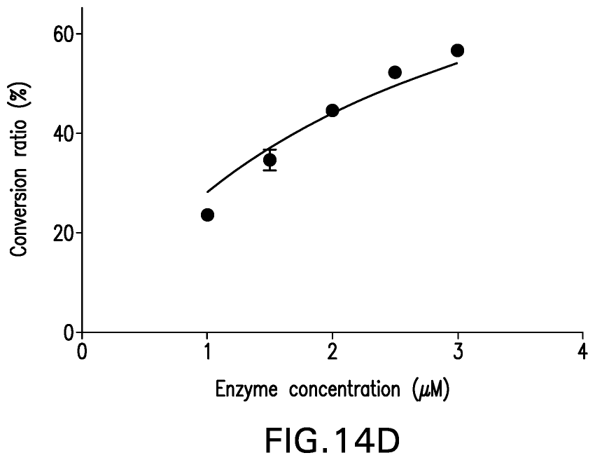

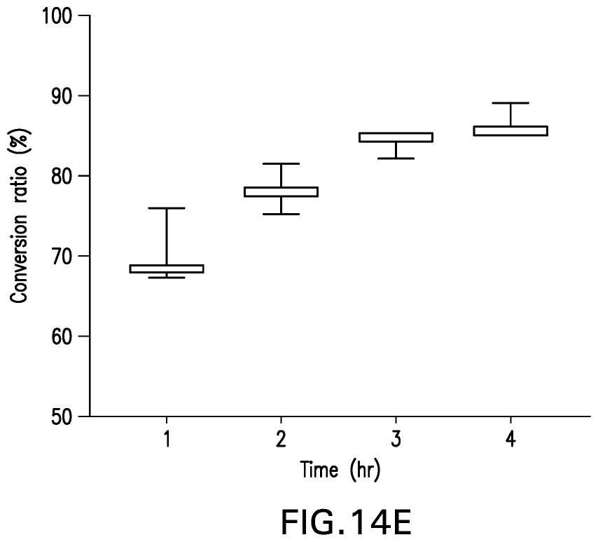

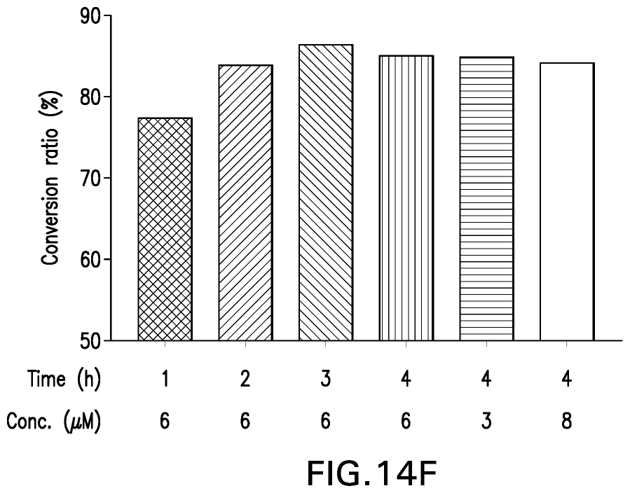

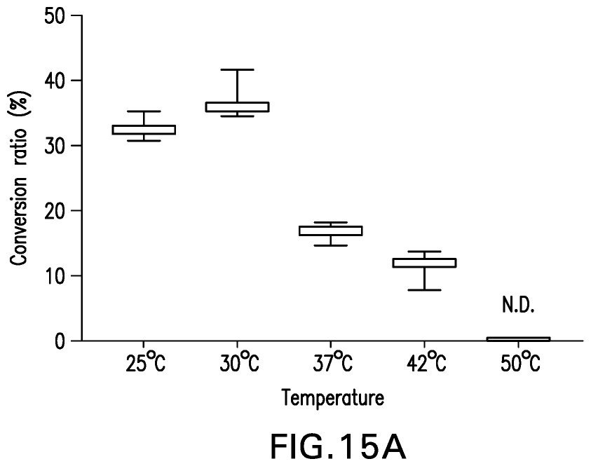

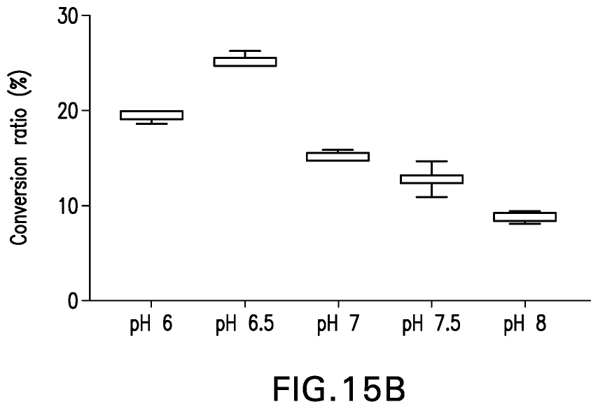

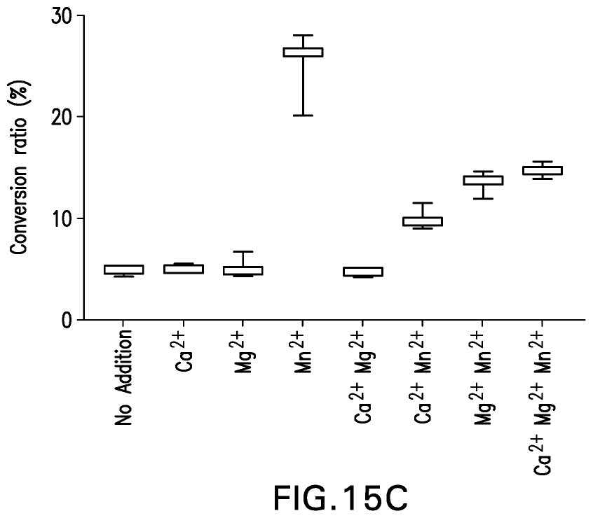

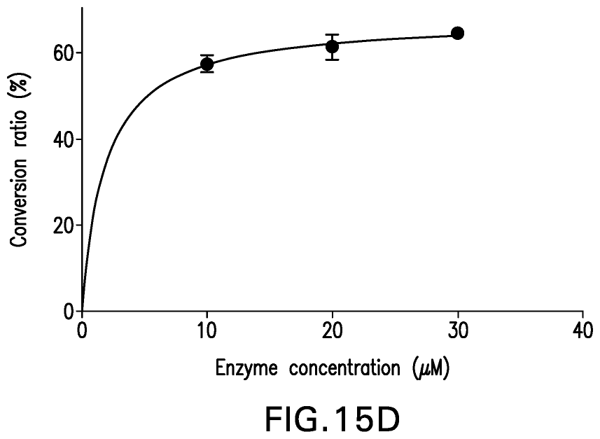

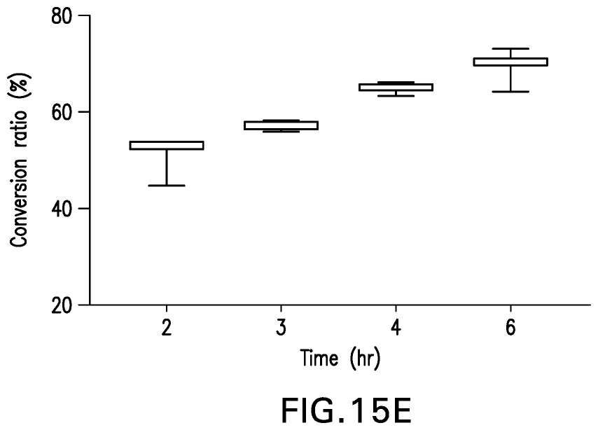

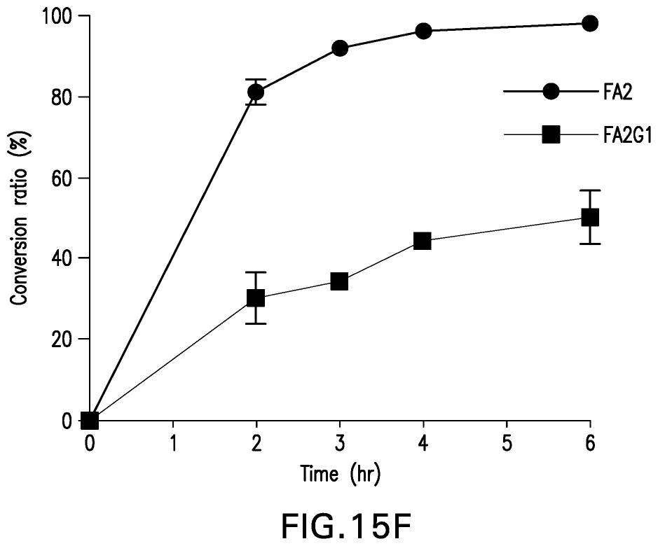

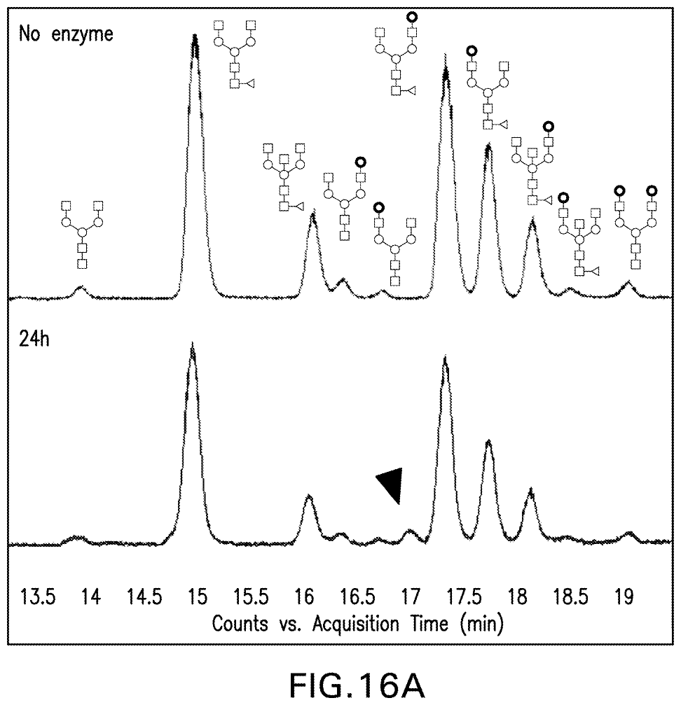

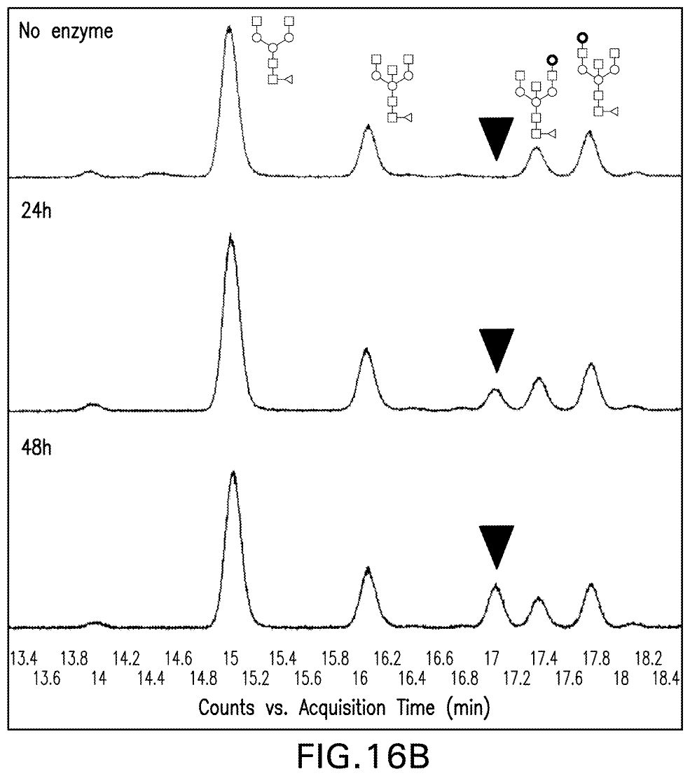

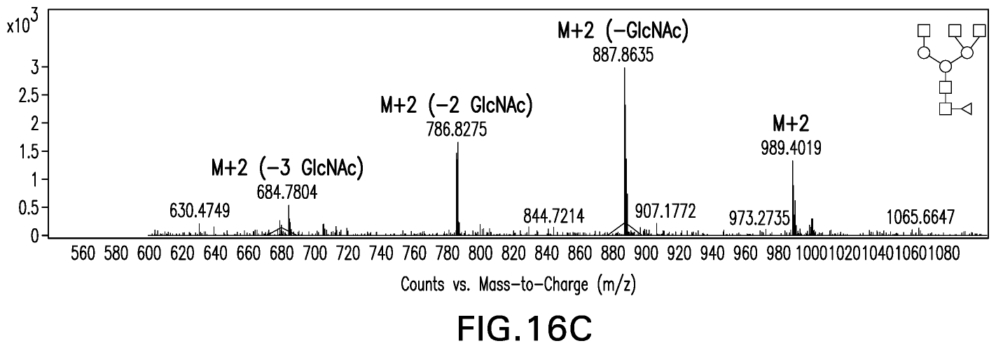

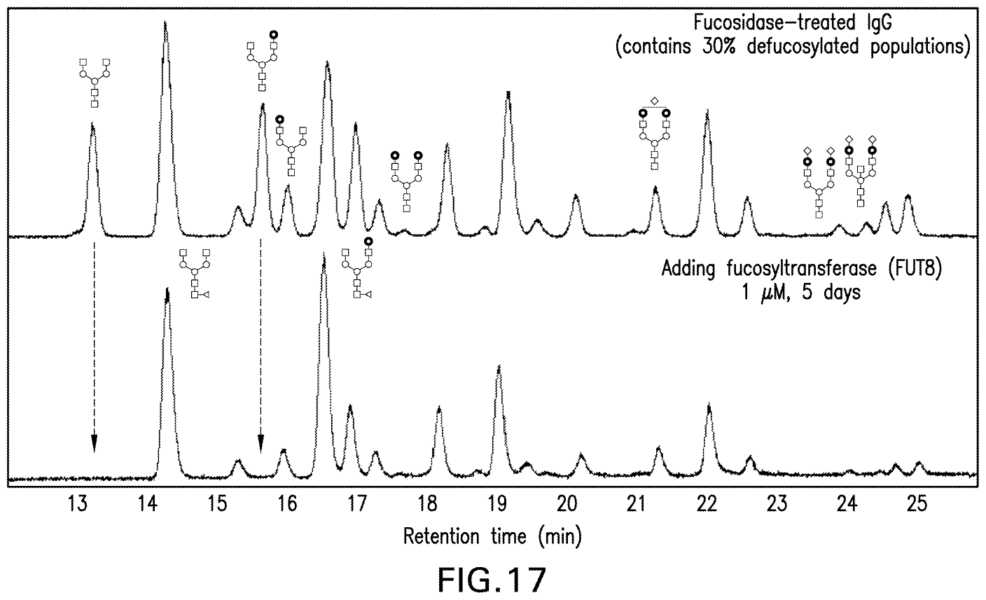

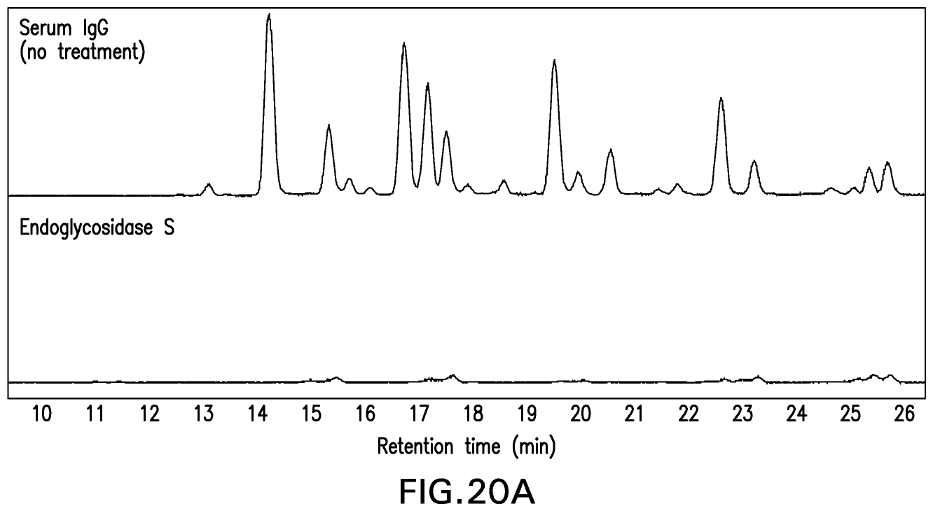

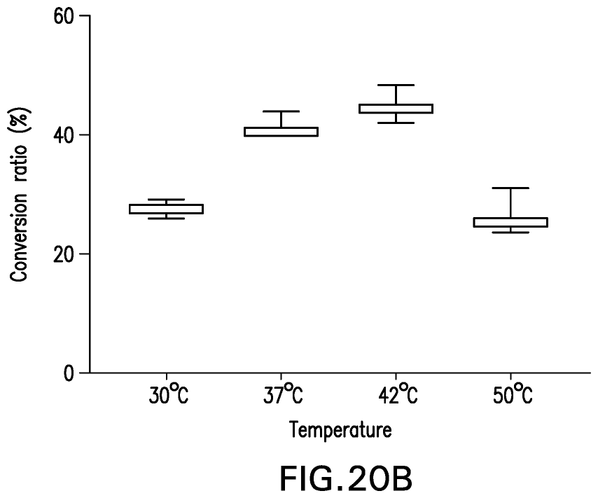

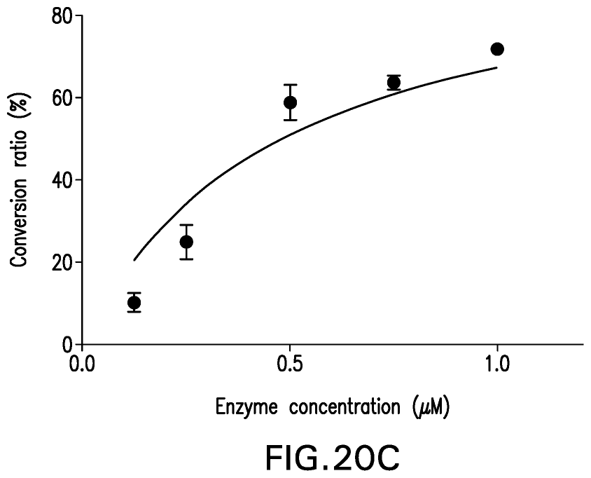

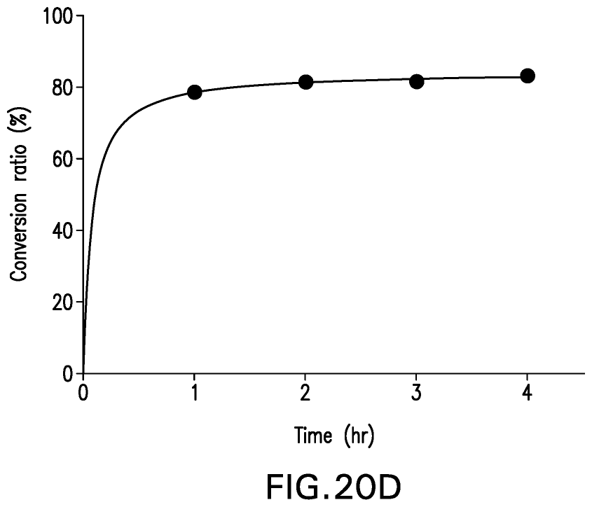

TABLE 3
Reaction conditions used for SPGR reactions in Examples 2and 3
Part 1

| Exp. | Enzyme (Amount) | Substrate | Buffer |
|---|---|---|---|
| FIG. 7A | Neuraminidase (2 μg) | Human serum IgG | 50 mM Sodium acetate |
| FIG. 7B | Neuraminidase | Human serum IgG | 50 mM Sodium acetate |
| FIG. 7C | Neuraminidase (16 μg) | Human serum IgG | 50 mM Sodium acetate |
| FIG. 2 | Neuraminidase (32 μg) | Human serum IgG | 50 mM Sodium acetate |
| FIG. 8A | Galactosidase S (12.5 μg) | Human serum IgG | 50 mM Sodium acetate |
| FIG. 8B | Galactosidase S | Human serum IgG | 50 mM Sodium acetate |
| FIG. 8C | Galactosidase S (75 μg) | Human serum IgG | 50 mM Sodium acetate |
| FIG. 2 | Galactosidase S (37.5 μg) | Human serum IgG | 50 mM Sodium acetate |
| FIG. 9A | N-Acetylglucosaminidase S (3 μg) | Human serum IgG | 50 mM Sodium acetate |
| FIG. 9B | N-Acetylglucosaminidase S | Human serum IgG | 50 mM Sodium acetate |
| FIG. 9C | N-Acetylglucosaminidase S (25 μg) | Human serum IgG | 50 mM Sodium acetate |
| FIG. 2 | N-Acetylglucosaminidase S (35 μg) | Human serum IgG | 50 mM Sodium acetate |
| FIG. 10A | Fucosidase O (1.32 mg) | FM3 IgG | 50 mM Sodium acetate |
| FIG. 10C | Fucosidase O (0.33 mg) | Human serum IgG | 50 mM Sodium acetate |
| FIG. 20A | Endoglycosidase S (27 μg) | Human serum IgG | 50 mM Sodium acetate |
| FIG. 20B | Endoglycosidase S (3.4 μg) | Human serum IgG | 50 mM Sodium acetate |
| FIG. 20C | Endoglycosidase S | Human serum IgG | 50 mM Sodium acetate |
| FIG. 20D | Endoglycosidase S (13.6 μg) | Human serum IgG | 50 mM Sodium acetate |
| FIG. 11A | α2-6 Sialyltransferase (1.1 μg) | Human serum IgG | 25 mM Tris-HCl |
| FIG. 11B | α2-6 Sialyltransferase (1.1 μg) | Human serum IgG | 25 mM Tris-HCl |
| FIG. 11C | α2-6 Sialyltransferase (1.1 μg) | Human serum IgG | 25 mM Tris-HCl |
| FIG. 11D | α2-6 Sialyltransferase | Human serum IgG | 25 mM Tris-HCl |
| FIG. 11E | α2-6 Sialyltransferase (44 μg) | Human serum IgG | 25 mM Tris-HCl |
| FIG. 2 | α2-6 Sialyltransferase (15 μg) | Human serum IgG | 25 mM Tris-HCl |
| FIG. 12A | β1-4 Galactosyltransferase 1 (10 μg) | Human serum IgG | 25 mM Tris-HCl |
| FIG. 12B | β1-4 Galactosyltransferase 1 (8 μg) | Human serum IgG | 25 mM Tris-HCl |
| FIG. 12C | β1-4 Galactosyltransferase 1 (4 μg) | Human serum IgG | 25 mM Tris-HCl |
| FIG. 12D | β1-4 Galactosyltransferase 1 | Human serum IgG | 25 mM Tris-HCl |
| FIG. 12E | β1-4 Galactosyltransferase 1 (30 μg) | Human serum IgG | 25 mM Tris-HCl |
| FIG. 2 | β1-4 Galactosyltransferase 1 (25 μg) | Human serum IgG | 25 mM Tris-HCl |
| FIG. 14A | N-Acetylglucosaminyltransferase 1 (1.2 μg) | FM3 IgG | 20 mM HEPES |
| FIG. 14B | N-Acetylglucosaminyltransferase 1 (1.2 μg) | FM3 IgG | 20 mM HEPES |
| FIG. 14C | N-Acetylglucosaminyltransferase 1 (2.4 μg) | FM3 IgG | 20 mM HEPES |
| FIG. 14D | N-Acetylglucosaminyltransferase 1 | FM3 IgG | 20 mM HEPES |
| FIG. 14E | N-Acetylglucosaminyltransferase 1 (15 μg) | FM3 IgG | 20 mM HEPES |
| FIG. 14F | N-Acetylglucosaminyltransferase 1 (30 μg) | FM3 IgG | 20 mM HEPES |
| FIG. 13A-B | N-Acetylglucosaminyltransferase 1 (20 μg) | FM3 IgG | 20 mM HEPES |
| FIG. 15A | N-Acetylglucosaminyltransferase 3 (9 μg) | Human serum IgG | 20 mM HEPES |
| FIG. 15B | N-Acetylglucosaminyltransferase 3 (9 μg) | Human serum IgG | 20 mM HEPES or MES |
| FIG. 15C | N-Acetylglucosaminyltransferase 3 (9 μg) | Human serum IgG | 20 mM MES |
| FIG. 15D | N-Acetylglucosaminyltransferase 3 | Human serum IgG | 20 mM MES |
| FIG. 15E | N-Acetylglucosaminyltransferase 3 (45 μg) | Human serum IgG | 20 mM MES |
| FIG. 2 | N-Acetylglucosaminyltransferase 3 (30 μg) | Human serum IgG | 20 mM MES |
| FIG. 16A-C | N-Acetylglucosaminyltransferase 5 (33 μg) | Human serum IgG | 20 mM HEPES |
| FIG. 17 | Fucosyltransferase (60 μg) | Defucosylated IgG | 100 mM MES |

Part 2

| | | Mol % | Weight % | Reaction | | | | |
|---|---|---|---|---|---|---|---|---|
| Exp. | Enzyme (Amount) | (E-to-S) | (E-to-S) | Time | | Temp | pH | Cation |
| FIG. 7A | Neuraminidase (2 μg) | 0.7% | 0.2% | 1 | h | Var. | 5.5 | Ca²⁺ |
| FIG. 7B | Neuraminidase | Var. | Var. | 1 | h | 42° C. | 5.5 | Ca²⁺ |
| FIG. 7C | Neuraminidase (16 μg) | 5.6% | 1.6% | Var. | | 42° C. | 5.5 | Ca²⁺ |
| FIG. 2 | Neuraminidase (32 μg) | 11.2% | 3.2% | 4 | h | 42° C. | 5.5 | Ca²⁺ |
| FIG. 8A | Galactosidase S (12.5 μg) | 0.75% | 1.25% | 1 | h | Var. | 5.5 | Ca²⁺ |
| FIG. 8B | Galactosidase S | Var. | Var. | 1 | h | 37° C. | 5.5 | Ca²⁺ |
| FIG. 8C | Galactosidase S (75 μg) | 4.5% | 7.5% | Var. | | 37° C. | 5.5 | Ca²⁺ |
| FIG. 2 | Galactosidase S (37.5 μg) | 2.25% | 3.75% | 16 | h | 37° C. | 5.5 | Ca²⁺ |
| FIG. 9A | N-Acetylglucosaminidase S (3 μg) | 0.37% | 0.3% | 1 | h | Var. | 5.5 | Ca²⁺ |
| FIG. 9B | N-Acetylglucosaminidase S | Var. | Var. | 1 | h | 42° C. | 5.5 | Ca²⁺ |
| FIG. 9C | N-Acetylglucosaminidase S (25 μg) | 3.2% | 2.5% | Var. | | 42° C. | 5.5 | Ca²⁺ |
| FIG. 2 | N-Acetylglucosaminidase S (35 μg) | 4.2% | 3.5% | 4 | h | 42° C. | 5.5 | Ca²⁺ |
| FIG. 10A | Fucosidase O (1.32 mg) | 400% | 132% | 3-days | | 37° C. | 4.5 | None |
| FIG. 10C | Fucosidase O (0.33 mg) | 100% | 33% | 5-days | | 37° C. | 4.5 | None |
| FIG. 20A | Endoglycosidase S (27 μg) | 3% | 2.7% | 4 | h | 42° C. | 5.5 | Ca²⁺ |
| FIG. 20B | Endoglycosidase S (3.4 μg) | 0.37% | 0.34% | 1 | h | Var. | 5.5 | Ca²⁺ |
| FIG. 20C | Endoglycosidase S | Var. | Var. | 1 | h | 42° C. | 5.5 | Ca²⁺ |
| FIG. 20D | Endoglycosidase S (13.6 μg) | 1.5% | 1.36% | Var. | | 42° C. | 5.5 | Ca²⁺ |
| FIG. 11A | α2-6 Sialyltransferase (1.1 μg) | 0.37% | 0.11% | 24 | h | Var. | 7.5 | Ca²⁺, Mn²⁺ |
| FIG. 11B | α2-6 Sialyltransferase (1.1 μg) | 0.37% | 0.11% | 24 | h | 37° C. | Var. | Ca²⁺, Mn²⁺ |
| FIG. 11C | α2-6 Sialyltransferase (1.1 μg) | 0.37% | 0.11% | 24 | h | 37° C. | 7.5 | Var. |
| FIG. 11D | α2-6 Sialyltransferase | Var. | Var. | 1 | h | 37° C. | 7.5 | Mg²⁺ |
| FIG. 11E | α2-6 Sialyltransferase (44 μg) | 14.8% | 4.4% | Var. | | 37° C. | 7.5 | Mg²⁺ |
| FIG. 2 | α2-6 Sialyltransferase (15 μg) | 5% | 1.5% | 16 | h | 37° C. | 7.5 | Mg²⁺ |
| FIG. 12A | β1-4 Galactosyltransferase 1 (10 μg) | 3.7% | 1% | 1 | h | Var. | 7 | Na⁺, Mn²⁺ |
| FIG. 12B | β1-4 Galactosyltransferase 1 (8 μg) | 3% | 0.8% | 2 | h | 50° C. | Var. | Na²⁺, Mn²⁺ |
| FIG. 12C | β1-4 Galactosyltransferase 1 (4 μg) | 1.5% | 0.4% | 3 | h | 50° C. | 7 | Var. |
| FIG. 12D | β1-4 Galactosyltransferase 1 | Var. | Var. | 1 | h | 50° C. | 7 | Mn²⁺ |
| FIG. 12E | β1-4 Galactosyltransferase 1 (30 μg) | 11.2% | 3% | Var. | | 50° C. | 7 | Mn²⁺ |
| FIG. 2 | β1-4 Galactosyltransferase 1 (25 μg) | 9.4% | 2.5 | 16 | | 50° C. | 7 | Mn²⁺ |
| FIG. 14A | N-Acetylglucosaminyl-transferase 1 (1.2 μg) | 0.37% | 0.12% | 4 | h | Var. | 7 | Na⁺, Mn²⁺ |
| FIG. 14B | N-Acetylglucosaminyl-transferase 1 (1.2 μg) | 0.37% | 0.12% | 4 | h | 37° C. | Var. | Na⁺, Mn²⁺ |
| FIG. 14C | N-Acetylglucosaminyl-transferase 1 (2.4 μg) | 0.74% | 0.24% | 2 | h | 30° C. | 7.5 | Var. |
| FIG. 14D | N-Acetylglucosaminyl-transferase 1 | Var. | Var. | 1 | h | 30° C. | 7.5 | Na⁺, Ca²⁺, Mg²⁺, Mn²⁺ |
| FIG. 14E | N-Acetylglucosaminyl-transferase 1 (15 μg) | 4.5% | 1.5% | Var. | | 30° C. | 7.5 | Na⁺, Ca²⁺, Mg²⁺, Mn²⁺ |
| FIG. 14F | N-Acetylglucosaminyl-transferase 1 (30 μg) | Var. | Var. | Var. | | 30° C. | 7.5 | Na⁺, Ca²⁺, Mg²⁺, Mn²⁺ |
| FIG. 13A-B | N-Acetylglucosaminyl-transferase 1 (20 μg) | 6% | 2% | 4 | | 30° C. | 7.5 | Na⁺, Ca²⁺, Mg²⁺, Mn²⁺ |
| FIG. 15A | N-Acetylglucosaminyl-transferase 3 (9 μg) | 1.5% | 0.9% | 24 | h | Var. | 7 | Na⁺, Mn²⁺ |
| FIG. 15B | N-Acetylglucosaminyl-transferase 3 (9 μg) | 1.5% | 0.9% | 24 | h | 37° C. | Var. | Na⁺, Mn²⁺ |
| FIG. 15C | N-Acetylglucosaminyl-transferase 3 (9 μg) | 1.5% | 0.9% | 24 | h | 30° C. | 6.5 | Var. |
| FIG. 15D | N-Acetylglucosaminyl-transferase 3 | Var. | Var. | 1 | h | 30° C. | 6.5 | Na⁺, Mn²⁺ |
| FIG. 15E | N-Acetylglucosaminyl-transferase 3 (45 μg) | 7.5% | 4.5% | Var. | | 30° C. | 6.5 | Na⁺, Mn²⁺ |
| FIG. 2 | N-Acetylglucosaminyl-transferase 3 (30 μg) | 5% | 3% | 16 | h | 30° C. | 6.5 | Na⁺, Mn²⁺ |
| FIG. 16A-C | N-Acetylglucosaminyl-transferase 5 (33 μg) | 7.5% | 3.3% | 24 | h | 30° C. | 7.5 | Na⁺, Mn²⁺ |
| FIG. 17 | Fucosyltransferase (60 μg) | 14% | 6% | 5 | days | 37° C. | 7 | Na⁺ |

Var. = Variable
Protein A resin (0.1 mL) was used for IgG (1 mg) immobilization for all the reactions. The final reaction volume was 0.1 mL. (F)M3 IgG was prepared from human serum IgG by applying sequential glycan remodeling using SPGR as described herein. Cation ion concentration: 5 mM for Ca²⁺ (CaCl₂), 50 mM for Na⁺ (NaCl) and 10 mM for Mg²⁺ and Mn²⁺ (MgCl₂, MnCl₂).

(V) Elution of glycoengineered IgG: 500 μL protein A-IgG elution buffer was added to the reaction columns in two portions with a two-minute incubation for each at room temperature. The eluent was collected into a 0.5 mL MWCO (molecular weight cut-off) tube and concentrated via centrifugation at 14000 g for five minutes, followed by buffer exchange into 50 mM HEPES buffer (pH 8). After adjusting the concentration of the eluted IgG substrate to 3 mg/mL using NanoDrop, the IgG substrates were stored at 4° C. and were ready for analysis. IgG concentration was adjusted in this step in order to ensure the same amount of sample was charged to the downstream analyses.

### LC-MS Analysis of IgG Glycans

This protocol is adapted from the Glycoworks manual provided by Waters.

(I) Glycan isolation: 7.5 μL IgG substrate (3 mg/mL, prepared as described above), 6 μL RapiGest SF (50 mg/ml, in Glycoworks buffer), and 15.3 μL water were mixed in a 1.5 mL microtube. The mixture was then incubated at 90° C. for five minutes to denature the substrates. After the samples were cooled down to room temperature, 1.2 μL Rapid PNGase F was added to the tube, followed by another incubation at 50° C. for 10 minutes.

(II) Glycan labeling: After PNGase F digestion, 12 μL RapiFluor-MS solution (70 mg/mL, in dimethylformamide (DMF)) was added to the solution. The mixture was gently vortexed and then incubated at room temperature for 20 minutes without any light exposure.

(III) Glycan purification: After labeling, the samples were diluted with 360 μL acetonitrile (1:9 volume ratio). Oasis SPE μPlate from Waters (along with the use of μPlate extraction manifold) was employed for the 1^{st} solid-phase extraction purification, and Discovery SPE from Sigma-Aldrich (along with the use of 20-well SPE vacuum manifold) was used for the 2^{nd} purification to ensure high signal-to-noise ratio: The SPE columns/μPlate were first washed by water (one column volume) and then conditioned by water-acetonitrile solution (10:90 v/v, one column volume). The glycan samples (in 90% acetonitrile solution) were then charged to the column/μPlate, followed by washing with washing buffer (formic acid/water/acetonitrile 1:9:90 v/v/v, two column volumes). The glycans were then eluted using 60 μL elution buffer (200 mM ammonium acetate in 5% acetonitrile).

(IV) HILIC-MS analysis: The purified glycan samples were injected to UPLC equipped with ACQUITY BEH Glycan column (130 Å, 1.7 μm, 2.1×150 mm) tandem with IMQ-TOF MS for glycan profile analysis. Refer to the literature published by Pucić et al. (Mol. Cell. Proteomics 10, M111.010090, doi: 10.1074/mcp.M111.010090 (2011)) and 1 Krištić et al. (J. Gerontol. A Biol. Sci. Med. Sci. 69, 779-789, doi: 10.1093/gerona/glt190 (2014)) for detailed peak assignment. The method provided by Waters for glycan chromatography used is shown in Table 4.

TABLE 4

| Mobile phase A: 50 mM ammonium format in H2O, pH 4.4 | | | |
|---|---|---|---|
| Mobile phase B: 100% Acetonitrile | | | |
| Temperature: 60° C. | | | |
| Injection volume: 5-10 μL | | | |
| Time (min) | Flow rate (mL/min) | % A | % B |
| 0 | 0.4 | 25 | 75 |
| 35 | 0.4 | 46 | 54 |
| 36.5 | 0.2 | 100 | 0 |
| 39.5 | 0.2 | 100 | 0 |
| 43.1 | 0.2 | 25 | 75 |
| 47.6 | 0.4 | 25 | 75 |
| 55 | 0.4 | 25 | 75 |

### Conversion Ratio Quantification

The conversion ratio of each SPGR reaction was calculated based on the consumption of the substrate glycan species. UV absorption at 260 nm (RapiFluor-MS) from chromatography analysis was used for the quantification of glycan populations. We first normalized the chromatographic peak area of the substrate glycan species to the total glycan peak area (Equation 1). This gives us the percentage of substrate glycan population. The reduction of the substrate glycan population after the reaction was then divided by the initial value to calculate the percentage of substrate conversion (Equation 2). We assume that there is no glycan shedding off from IgG during the experiments (namely, no endoglycosidase activity). Refer to Table 5 for detailed substrate species used in the calculation.

TABLE 5

| | | Substrate glycan species |
|---|---|---|
| | | (Acceptor) |
| | | (excluding low-abundant, non- |
| Enzyme | Donor | detectable glycoforms) |
| Neuraminidase | N.A. | (F)A2(B)G2S2, (F)A2(B)G2S1, |
| | | (F)A2(B)G1S1 |
| Galactosidase S | N.A. | (F)A2(B)G2S1, (F)A2(B)G2, |
| | | (F)A2(B)G1 |
| N-Acetylglucos- | N.A. | (F)A2(B)G1, (F)A2(B) |
| aminidase S | | |
| Fucosidase O | N.A. | All fucosylated IgG glycans |
| Endoglycosidase S | N.A. | All IgG glycans |
| α2-6 Sialyltransferase | Cytidine-5′- | (F)A2(B)G2, (F)A2(B)G1, |
| | monophospho-N- | (F)A2(B)G2S1 |
| | acetylneuraminic acid | |
| | (CMP-NANA) | |
| β1-4 | Uridine 5′- | (F)A2(B), (F)A2(B)G1 |
| Galactosyltransferase 1 | diphosphogalactose | |
| | (UDP-Gal) | |
| N-Acetylglucosaminyl- | Uridine 5′-diphospho- | (F)M3 (glycoengineered IgG) |
| transferase 1 | N-acetylglucosamine | |
| | (UDP-GlcNAc) | |
| N-Acetylglucosaminyl- | Uridine 5′-diphospho- | (F)A2, (F)A2G1 |
| transferase 3 | N-acetylglucosamine | |
| | (UDP-GlcNAc) | |
| Fucosyltransferase | Guanosine 5′- | M3 (glycoengineered IgG) |
| | diphospho-β-L-fucose | |
| | (GDP-Fuc) | |

Saccharide donor and acceptors in each enzymatic reaction. The substrate glycan species (acceptors) were listed based on our observations.

For endoglycosidase reactions, the activity was quantified by absolute glycan quantification using an internal standard. This is because endoglycosidases' activities result in the reduction of all glycan signals in the chromatography analyses. A known amount of internal standard (Glycan quantitative standard, Waters) was added to the sample to measure the amount of IgG glycan before and after the reactions.

$$\\ \begin{matrix}Substrate glycan population = \frac{\sum Peak {area}_{substrate 1 , 2.3 … i}}{\sum Peak {area}_{all glycans}} = {R}_{s} & ( Equation 1 )\end{matrix}$$

$$\\ \begin{matrix}Conversion Ratio = \frac{{R}_{S ( initial )} - {R}_{S ( final )}}{{R}_{s ( initial )}} & ( Equation 2 )\end{matrix}$$

### CR₅₀ Calculation

CR₅₀ is defined as the enzyme-to-substrate (IgG) molar ratio that leads to 50% substrate species conversion into the products in one hour at optimized working conditions (temperature, pH, cation) using SPGR. This value is determined based on dose-dependent experiments where the conversion ratio at different enzyme concentrations in one hour was tested. A sigmoidal curve fitting ([agonist] vs normalized response, GraphPad Prism, see the equation below) was then applied to the data for calculating the enzyme concentration that gives 50% substrate conversion. The resulting enzyme concentration is divided by the substrate (IgG) concentration to give CR₅₀.

$$\\ Y = \frac{100 X}{{CR}_{50} + X} ,$$

where Y is normalized response from 0 to 100; X is the concentration of enzyme.

### Physical Property Characterization of SPGR-Engineered IgGs

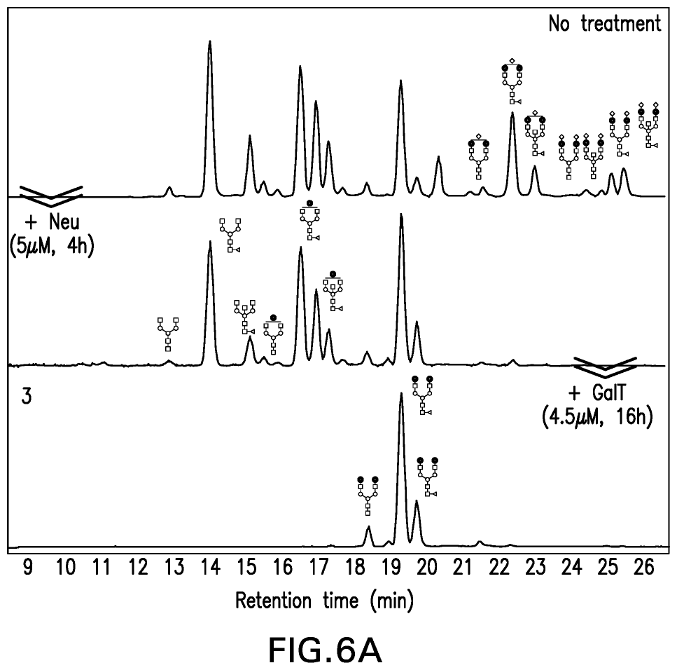

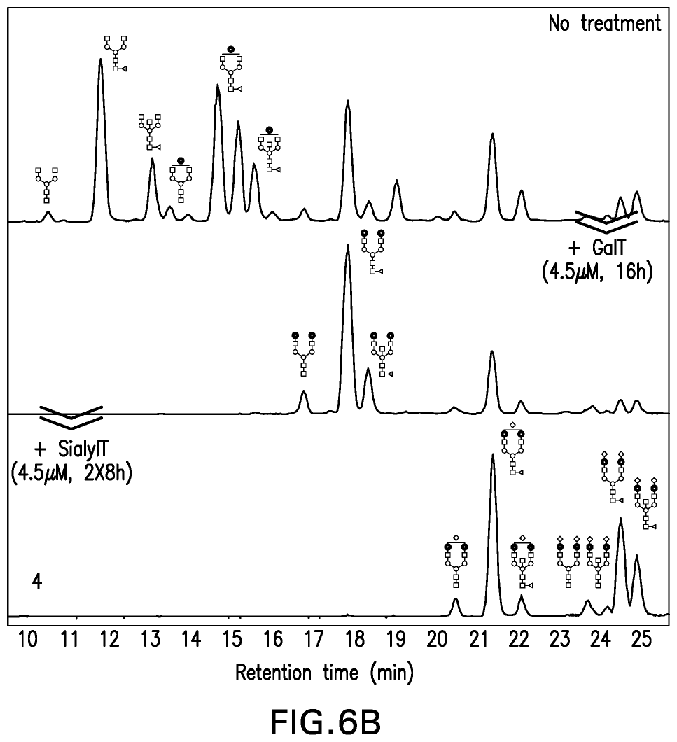

Dynamic light scattering (DLS), melting temperature (Tₘ) and aggregation temperature (T_{agg}) studies were executed using UNcle (Unchainedlabs). Glycoengineered IgGs from SPGR reactions were eluted from protein A resins, followed by buffer exchange into HEPES buffer as described above. The protein concentration was then adjusted to 1 mg/ml using NanoDrop. Then, 9 μL of the purified IgG samples were injected into UNcle sample holders (five replicates for each sample). DLS measurement was performed at 25° C. (four acquisitions, five seconds each). Static light scattering (SLS) for Tₘ and T_{agg} measurement was carried out from 25° C. to 90° C. with a temperature increase of 0.3° C. per minute. All the tested IgG samples (1-10) contained an about 5% defucosylated population. The bi-antennary N-glycan samples (2-4) had about 10% bisecting glycoforms. The hybrid N-glycan samples (5-7) had about 10% (F)M3 glycans due to the reversible activity of GnT-I. Sialylated N-glycan samples (4 & 10) possessed about 1:1 mono- and bi-sialylated N-glycan populations. Refer to FIG. 3A-3C and FIG. 6A-6B for detailed N-glycan species and population distributions.

### Binding Assays Between Glycoengineered IgGs and Fc Gamma Receptor 1

This protocol is adapted from the AlphaLISA human FCGR binding kit manual provided by PerkinElmer. Briefly, serial dilutions of SPGR-engineered IgGs with 1× HiBlock buffer were prepared with the highest concentration at 1 mg/mL and the lowest concentration at 0.1 μg/mL. Then, 10 μL of each diluted IgG samples were mixed with 10 μL 4× human FcγRI solution and 20 μL Donor/acceptor beads solution into a white 96-well plate. The plate was then sealed and incubated at 25° C. for 90 minutes without any light exposure. After the reaction, the fluorescence signal at 615 nm was determined using an EnVision Multimode Plate Reader (equipped with AlphaScreen module).

### Computational Modeling of IgG-Protein a Complex

Protein A homology model was constructed using the Swiss-Model server (Waterhouse et al., Nucleic Acids Res. 46, W296-w303, doi: 10.1093/nar/gky427 (2018)). PDB structure 5H7B, which has 79.5% sequence identity with Protein A, was used as a template to construct the homology model (Youn et al., Sci. Rep. 7, 2595, doi: 10.1038/s41598-017-02803-z (2017)). A visual inspection of Protein A model illustrated four distinct IgG binding domains. PDB structure 5U4Y was used as a template to identify the spatial positioning of the full-length Protein A and the IgG (Ultsch et al., Protein Eng. Des. Sel. 30, 619-625, doi: 10.1093/protein/gzx029 (2017)). The template structure contains only the B-domain of the protein A molecule. The spatial position of the B-domain helped us overlay the full-length protein-A molecule and allowed us to identify steric hindrances between other protein-A domains and the IgG molecule. Structure overlay and the movie illustrating clashes between the molecules was generated using Pymol (Molecular Graphics System, Version 2.0, Schrödinger LLC).

### Plotting and Graphic

Data plotting and curve fitting were done by using GraphPad Prism 8. Figures and cartoons were created by Adobe Illustrator.

### Example 2

We use human IgG as the substrate in this study because it is a major class of glycoproteins that have been applied in therapeutic development (Kaplon & Reichert, MAbs 11:219-238, doi: 10.1080/19420862.2018.1556465 (2019); Lu et al., J. Biomed. Sci. 27: doi: 10.1186/s12929-019-0592-z (2020)). We quantitatively examined more than 30 glycan engineering enzymes for their activities on intact IgG immobilized on resins and then applied them in SPGR. This method has allowed us to harmonize IgG glycans into ten different glycoforms, including non-canonical structures, in 48 hours with an averaged conversion ratio greater than 95%. Physical and biochemical analyses indicated that the SPGR-engineered IgGs preserved integrity and functionality, suggesting that SPGR has high biocompatibility to the substrates.

### The Design of Solid-Phase Glycan Remodeling (SPGR) for IgG Glycoengineering

Our strategy to achieve efficient, successive glycan remodeling is immobilizing IgG onto protein A resins and then executing enzymatic reactions heterogeneously (FIG. 1A). This enables product purification by filtration, greatly speeding up multi-step reactions. We use empty SPE (solid-phase extraction) columns with standard Luer fittings as the SPGR reaction vessels. The Leur fittings can be connected to either syringes or vacuum manifolds to control the flow speed during washing processes. A frit is inserted into the bottom of the column for trapping the solid supports. SPGR processes can be separated into five steps: 1) resin loading: 2) IgG immobilization: 3) washing and conditioning: 4) enzymatic glycan remodeling, and: 5) elution and downstream analyses. The third and fourth steps are repeated for successive reactions until the remodeling is complete.

To identify capable glycosylation enzymes for SPGR, we quantitatively analyzed the activity of 34 candidates, including exoglycosidases, endoglycosidases, and glycosyltransferases (FIG. 1B, Tables 1A-C). Each enzyme was incubated with immobilized IgG for one or 24 hours. The enzyme activity-indicated by the consumption of substrate glycan species—was then quantified via chromatographic analysis. The candidates with the highest activity in each enzyme class were selected for SPGR applications and their working conditions were further optimized (Table 2, FIGS. 7A-21C). Also, we defined CR₅₀ to be the enzyme-to-substrate (IgG) ratio that leads to 50% substrate conversion into the products in one hour using SPGR (Table 2). This value allows us to estimate how much enzyme is required for SPGR reactions when the amount of substrate varies. It also provides the information about the reaction efficiency between different enzymes: the smaller the CR₅₀ value, the more efficient is the reaction.

Trimming IgG Glycans with Glycosidases

IgGs have two highly conserved glycosylation sites on the crystallizable region (Fc) at Asn 297 where more than 20 complex-type glycoforms have been found with the majority in bi-antennary structures (FIG. 1C) (Vidarsson et al., Frontiers in Immunology 5, doi: 10.3389/fimmu.2014.00520 (2014); Pucić et al., Mol. Cell. Proteomics 10, M111.010090, doi: 10.1074/mcp.M111.010090 (2011)). IgG glycans play essential roles in Fc receptor (FcR)-mediated activities, such as antibody-dependent cellular cytotoxicity (ADCC) (Huhn et al., Proteomics 9, 882-913, doi: 10.1002/pmic.200800715 (2009); Li et al., Proc. Natl. Acad. Sci USA 114, 3485-3490, doi: 10.1073/pnas. 1702173114 (2017); Bruhns & Jönsson, Immunol. Rev. 268, 25-51, doi: 10.1111/imr.12350 (2015)). About 20% of IgG glycans contain terminal sialic acids through α2-6 linkages. These sialylated glycans have been known to confer anti-inflammatory activity (Gudelj et al., Cell. Immunol. 333, 65-79, doi: https://doi.org/10.1016/j.cellimm.2018.07.009 (2018)). Similarly, IgG galactosylation modulates inflammatory properties and about 70% of the IgG glycans contain terminal β1-4 galactoses (Reily et al., Nature Reviews Nephrology 15, 346-366, doi: 10.1038/s41581-019-0129-4 (2019); Reiding et al., Front Med (Lausanne) 4, doi: 10.3389/fmed.2017.00241 (2017); Malhotra et al., Nature Medicine 1, 237-243, doi: 10.1038/nm0395-237 (1995)). The galactosylation level is also known to influence the clearance rate of glycoproteins in serum, mediated by asialoglycoprotein receptors, resulting in a direct impact on their pharmacokinetic properties (Stockert, Physiol Rev 75, 591-609, doi: 10.1152/physrev. 1995.75.3.591 (1995); Higel et al., Eur. J. of Pharma. and Biopharma. 139, 123-131, doi: https://doi.org/10.1016/j.ejpb.2019.03.018 (2019)). Compared to sialic acid and galactose, our understanding of N-Acetylglucosamine (GlcNAc)'s impacts on IgG is more limited. GlcNAc exists in all N-glycans and plays decisive roles in glycan biosynthesis pathways. Extended from the chitobiose core, GlcNAc glycosidic linkage serves as a watershed that determines the subclasses of N-glycans; complex-type, high-mannose, and hybrid-type N-glycans (Hossler et al., Glycobiology 19, 936-949, doi: 10.1093/glycob/cwp079 (2009)). Complex-type glycans can further branch into bisecting, bi-antennary, tri-antennary, and tetra-antennary glycans, and so on (Varki et al. in Essentials of Glycobiology, Cold Spring Harbor Laboratory Press, The Consortium of Glycobiology Editors, La Jolla, CA (2015)). About 90% of the IgG glycans are bi-antennary while the rest of them have bisecting structures (Pucić et al., Mol. Cell. Proteomics 10, M111.010090, doi: 10.1074/mcp.M111.010090 (2011)).

To control IgG glycoforms, we first aimed to harmonize them into the core saccharides by removing terminal sialic acid, galactose, and then GlcNAc. Neuraminidase (Neu, or sialidase) is a class of enzymes that cleaves the glycosidic linkages of sialic acids. Our screening showed that Neu from Clostridium perfringens has the highest activity on immobilized IgG with a CR₅₀ of 1%. This enzyme has a broad substrate spectrum and can function on all the IgG glycoforms containing terminal sialic acid (FIG. 2, FIGS. 7A-7C). Next, galactosidase (Gal) from Streptococcus pneumoniae showed the highest activity in our screening (CR₅₀=4.7%) for removing galactose (FIGS. 8A-8C). It functions on all the IgG glycoforms containing terminal galactoses with an optimal temperature at 37° C. To trim off GlcNAc, N-Acetylglucosaminidase (GlcNAcase) from S. pneumoniae showed the highest activity (CR so 0.4%, FIG. 9A-9C). It has low glycosidic linkage selectivity and can trim terminal GlcNAc extended from the chitobiose core (FIG. 2). Sequential treatments using these three enzymes leads to IgG glycan harmonization into (F)M3 structures (FIG. 3A).

Fucose on IgG glycan chitobiose core has been known to modulate IgG binding affinity to Fc receptors (Ferrara et al., Proc Natl Acad Sci USA 108, 12669-12674, doi: 10.1073/pnas. 1108455108 (2011); et al., Genes Cells 16, 1071-1080, doi: 10.1111/j.1365-2443.2011.01552.x (2011)). Defucosylated IgG has been reported to have an over 50-fold increase in ADCC activity (Shields. et al., J Biol Chem 277, 26733-26740, doi: 10.1074/jbc.M202069200 (2002)). As such a strong regulator, controlling the level of IgG core fucose has become an attractive strategy for improving the efficacy of IgG-based drugs. Over 90% of the human serum IgG glycans are fucosylated (Pucić et al., Mol. Cell. Proteomics 10, M111.010090, doi: 10.1074/mcp.M111.010090 (2011)). To identify the enzymes that can trim fucose from intact IgGs in their native confirmations, we tested seven fucosidases (Fuc). Unfortunately, none of them showed an acceptable activity (Table 1). It has been reported that Fuc only functions on intact IgG when their glycans are trimmed down to the GlcNAc-fucose disaccharides, which indicates a strong steric interference between the Fuc-fucose interaction (Huang et al., J. Am. Chem. Soc. 134, 12308-12318, doi: 10.1021/ja3051266 (2012)). Inspired by works from Huang et al., we tested the Fuc panel with glycoengineered IgG bearing (F)M3 glycans, as prepared above. The enzyme from Candidatus omnitrophica showed significantly improved activity on this group of substrates (FIGS. 10A-10D). A 20% conversion was achieved in a 3-day reaction. The conversion ratio was further increased to 65% if non-immobilized substrates were used.

Building IgG Glycans with Glycosyltransferases

Glycosyltransferases catalyze the transfer of saccharide(s) from activated sugar phosphates, the glycosyl donors, to glycosyl acceptor molecules, such as glycoproteins (Breton et al., Glycobiology 16, 29R-37R, doi: 10.1093/glycob/cwj016 (2005)). Sialyltransferase (SialyIT) from Homo sapiens exhibited the highest activity in our screening for installing sialic acid through α2-6 linkage to the IgG with terminal galactose. This enzyme has a relatively high CR so of 15.2% with an apparent substrate selectivity, as shown in FIG. 2 and FIGS. 11A-11G. Di-galactosylated glycan (FA2G2) and mono-galactosylated glycan with galactose at the α1-3 arm (FA2[3]G1) were completely transformed after a 16-hours reaction; while mono-galactosylated glycans at the α1-6 arm (FA2[6]G1) showed only minimal sialylation. The selective sialylation observed here agreed with previous reports and was likely caused by the folded conformation that the Fc region adopts when the galactose on the α1-6 arm is present (Harbison et al., Glycobiology 29, 94-103, doi: 10.1093/glycob/cwy097 (2018); Barb et al., Biochemistry 48, 9705-9707, doi: 10.1021/bi901430h (2009)). Besides, we also observed a decreased enzyme activity when the (F)A2G2 glycans were mono-sialylated (FIGS. 11A-11G).

To install galactose on IgG glycans, we selected the galactosyltransferase (GalT) from Homo sapiens (FIG. 2, FIGS. 12A-12E) (Amado et al., Biochimica et Biophysica Acta (BBA)—General Subjects 1473, 35-53, doi: https://doi.org/10.1016/S0304-4165 (99) 00168-3 (1999)). This enzyme catalyzed the transfer of galactose from Uridine 5′-diphosphogalactose (UDP-Gal) to IgG glycans with terminal GlcNAc. It has a CR₅₀ of 7% and a broad spectrum of substrate specificity that enables the transformation of all the non- and mono-galactosylated IgG glycans into bi-galactosylated forms.

The addition of GlcNAc to the chitobiose core is relatively complicated because this process involves a series of N-Acetylglucosaminyltransferases (GnT) with various substrate specificities (FIG. 1D) (Okada et al., Glycoscience: Biology and Medicine (eds Naoyuki Taniguchi et al.) 1163-1171 (Springer Japan, 2015)). We investigated the activity of five human GnTs that are responsible for complex N-glycan synthesis and obtained positive results from GnT-I, III, and V. GnT-I (MGAT1) initiates the formation of the complex-type and hybrid N-linked glycans by installing an β1-2 GlcNAc to the α1-3 mannose (FIGS. 13A-13B) (Fujiyama et al., Journal of Bioscience and Bioengineering 92, 569-574, doi: https://doi.org/10.1016/S1389-1723 (01) 80318-2 (2001); Yip et al., Biochem J. 321 (Pt 2), 465-474, doi: 10.1042/bj3210465 (1997)). We observed a good CR₅₀ of 3.8% in our activity screening, calculated based on (F)M3 glycan consumption. This enzyme likely possesses GlcNAcase activity as well because the conversion ratio reached a plateau of ˜85% (FIGS. 14A-14F) in all the conditions we tested. Whether or not its GlcNAcase activity can be repressed through genetic engineering to reach full conversion remains to be investigated. Human GnT-III (MGAT3) serves to install the bisecting GlcNAc to the β1-4 mannose through a β1-4 linkage (Narasimhan, J Biol Chem 257, 10235-10242 (1982)).

A higher level of bisecting GlcNAc on IgG results in enhanced ADCC activity and immune cells effector functions (Davies et al. Biotechnology and Bioengineering 74, 288-294, doi: 10.1002/bit.1119 (2001)). Reactions using serum IgG as the substrate suggested that human GnT-III can function on IgG glycoforms containing at least one terminal GlcNAc (FIG. 2). Moreover, it showed much higher activity on glycans with two terminal GlcNAcs as opposed to a single GlcNAc (FIGS. 15A-15F).

Tri- and tetra-antennary N-glycans are not typically reported on native human serum IgG, and were not observed in our studies. Human GnT-V (MGAT5) is reported to add the secondary GlcNAc to the α1-6 mannose through β1-6 linkage and leads to the formation of tri-/tetra-antennary glycoforms (Kim et al. Molec. Cell. Proteomics 7, 1-14, doi: 10.1074/mcp.M700084-MCP200 (2008); Shoreibah et al., J Biol Chem 268, 15381-15385 (1993)). We observed GnT-V activity after a 24-hours reaction with intact IgG, revealed by the formation of tri-antennary species. (FIGS. 16A-16C). The activity of GnT-V on intact IgG is low but potentially can be improved through genetic control or evolution for future applications.

Finally, mammalian 1,6-fucosyltransferase (FucT) catalyzes the transfer of a fucose residue from GDP-fucose, the donor substrate, to the reducing-end terminal GlcNAc residue through an α1,6-linkage (Ihara et al., Glycobiology 16, 333-342, doi: 10.1093/glycob/cwj068 (2005)). The activity of FucT on intact IgG was observed but the conversion ratio was low, likely due to the strong steric hindrance on the substrate. We further boosted the FucT reaction by increasing both enzyme concentration and incubation time. The result indicated that FucT prefers IgGs bearing A2 and A2G1 glycoforms (FIG. 17). Our data suggested that, by employing Fuc and FucT, modulating the level of core fucose on intact IgG is feasible.

### Reconstructing a Harmonized Glycosylation Profile

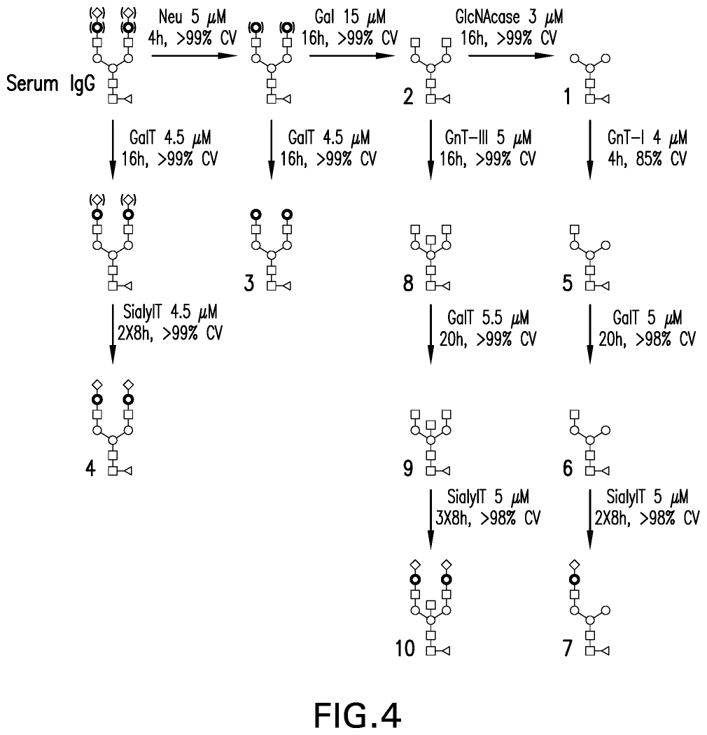

It was described how a Neu reaction followed by Gal treatment turned IgG glycans into GlcNAc-terminating glycoforms (FIG. 3A). We can further run a GlcNAcase reaction to trim the glycans into the core structures terminating with mannose. The whole sequence was performed in 24 hours without isolating IgG between steps. Similarly, remodeling using Neu and then GalT generated glycans with terminal galactose (FIG. 6A); while a GalT reaction followed by SiaT resulted in mono- and bi-sialylated species (FIG. 6B). The demonstrated SPGR routes in this work are summarized in FIG. 4.

IgG bearing (F)M3 glycans can also serve as starting materials for rebuilding non-canonical glycoforms. Starting with the (F)M3 glycans, we applied GnT-I, GalT, and then SiaT reactions to construct a series of hybrid species (FIG. 3B). Hybrid N-glycans are rare in nature and their effects on protein biology remain elusive. Whether they can be utilized for regulating the interactions between IgG and FcR is a great research topic of interest. In addition to hybrid species, we also thought to increase the population of bisecting N-glycans on IgG. We trimmed off terminal sialic acids and galactose from IgG and then introduced GnT-III to the resulting (F)A2 glycans. After overnight incubation at 5 μM, we reached a full conversion of IgG glycans into the bisecting forms (FIG. 3C). These bisecting glycans can also be further galactosylated or sialylated using SPGR. Compared to hybrid species, both GalT and SiaT showed slightly decreased activity on bisecting glycans. Therefore, a higher enzyme concentration or longer incubation time is required to achieve full conversion. The preparation of synthetic glycans with complex structures, traditionally, not only requires extensive experience but also a lot of time and effort. SPGR allowed us to reconstruct IgG N-glycans into the hybrid and bisecting glycoforms within 2-3 days, presenting a very efficient strategy for glycoengineering.

### SPGR is Biocompatible

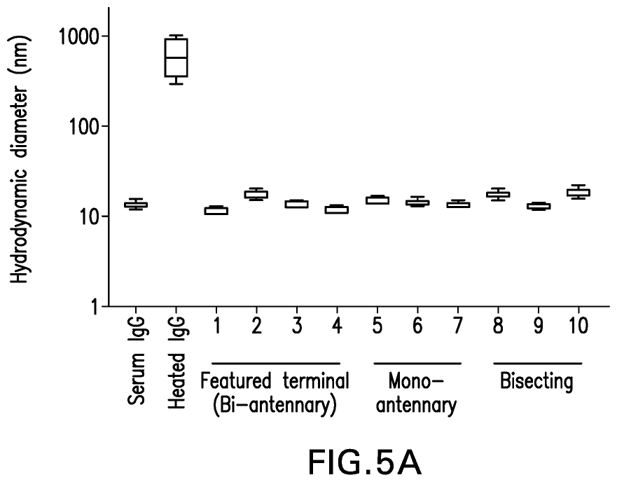

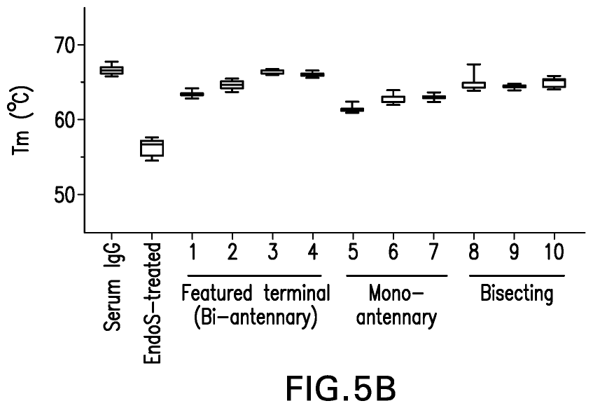

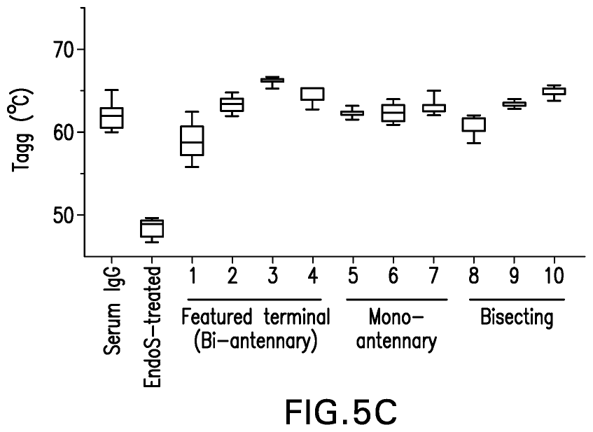

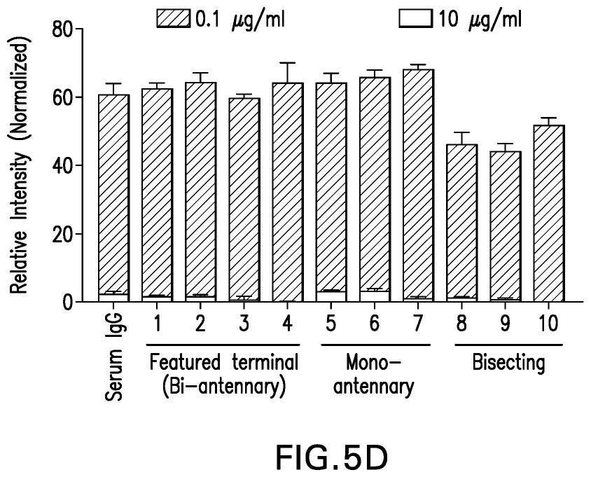

To examine the biocompatibility of SPGR to the substrates, we analyzed the physical properties of the glycoengineered IgGs prepared above. Size analyses using dynamic light scattering (DLS) showed no significant difference between native serum IgG and SPGR-engineered IgGs, suggesting that there was no denaturing and/or aggregation occurred during the remodeling processes (FIG. 5A). On the other hand, slight changes in their melting temperature (Tₘ) and aggregation temperature (T_{agg}) were observed (FIG. 5B-5C. Table 5).

TABLE 5
Physical and biochemical properties of SPGR-engineered IgGs

| | Serum | Endo S- | Fuc- | Featured terminal (Bi-antennary) | | | | Hybrid | | | Bisecting | | |
|---|---|---|---|---|---|---|---|---|---|---|---|---|---|
| | IgG | treated | treated | 1 | 2 | 3 | 4 | 5 | 6 | 7 | 8 | 9 | 10 |
| Hydro- | 13.3 ± | — | — | 11.5 ± | 17.7 ± | 13.6 ± | 11.8 ± | 14.8 ± | 14.2 ± | 13.3 ± | 17.4 ± | 12.8 ± | 18 ± |
| dynamic | 1.2 | | | 0.5 | 2 | 0.4 | 0.6 | 0.7 | 0.9 | 0.9 | 1.2 | 0.4 | 2.4 |
| diameter | | | | | | | | | | | | | |
| (nm) | | | | | | | | | | | | | |
| Melting | 66.7 ± | 56.4 ± | — | 63.4 ± | 64.8 ± | 66.3 ± | 66.1 ± | 61.4 ± | 62.9 ± | 63 ± | 64.8 ± | 64.5 ± | 65 ± |
| temperature | 0.7 | 1.4 | | 0.4 | 0.7 | 0.3 | 0.2 | 0.5 | 0.7 | 0.4 | 1.1 | 0.3 | 0.7 |
| (° C.) | | | | | | | | | | | | | |
| Aggregation | 62 ± | 48. 6 ± | — | 59 ± | 63.3 ± | 66.1 ± | 64.3 ± | 62.2 ± | 62.2 ± | 63 ± | 60.9 ± | 63.3 ± | 64.9 ± |
| temperature | 1.7 | 1.3 | | 2.3 | 1 | 0.5 | 1.1 | 0.5 | 1.2 | 0.9 | 1.2 | 0.4 | 0.6 |
| (° C.) | | | | | | | | | | | | | |
| FcγR I | 1.9 ± | — | 1.6 ± | 2.2 ± | 2.3 ± | 1.7 ± | 2 ± | 2.3 ± | 2.5 ± | 2.4 ± | 0.8 ± | 0.9 ± | 1.2 ± |
| binding | 0.4 | | 0.4 | 0.7 | 0.5 | 0.5 | 0.2 | 0.3 | 0.2 | 0.3 | 0.1 | 0.1 | 0.3 |
| (EC50, | | | | | | | | | | | | | |
| 10⁻⁷ g/mL) | | | | | | | | | | | | | |

We reasoned these changes were attributed to the altered structure of IgG. It has been known that the Asn297 glycans play roles in maintaining the conformation and stability of the Fc region through intra-molecular interactions (Krapp et al., Journal of Molecular Biology 325, 979-989, doi: https://doi.org/10.1016/S0022-2836 (02) 01250-0 (2003); Fang et al., Biochemistry 55, 860-868, doi: 10.1021/acs.biochem.5b01323 (2016); Mimura et al., Mol Immunol 37, 697-706, doi: 10.1016/s0161-5890 (00) 00105-x (2000)). Decreased Tₘ and T_{agg} values were observed in IgGs terminating with mannose (Varki et al. in Essentials of Glycobiology, Cold Spring Harbor Laboratory Press, The Consortium of Glycobiology Editors, La Jolla, CA (2015)) or GlcNAc (Spiro, Glycobiology 12, 43R-56R, doi: 10.1093/glycob/12.4.43R (2002); Reily et al., Nature Reviews Nephrology 15, 346-366, doi: 10.1038/s41581-019-0129-4 (2019); van de Bovenkamp et al., Journal of Immunology 196, 1435-1441, doi: 10.4049/jimmunol. 1502136 (2016)), the glycoforms showing reduced intra-molecular interactions (Krapp et al., Journal of Molecular Biology 325, 979-989, doi: https://doi.org/10.1016/S0022-2836 (02) 01250-0 (2003)). Similarly, our hybrid glycans (5-7) had lower Tₘ values compared to others. These glycoforms lack the α1-6 arm which is important for forming intra-molecular interaction within the Fc region (Harbison et al., Glycobiology 29, 94-103, doi: 10.1093/glycob/cwy097 (2018). As expected, the removal of the bulk of the glycan structures using Endo S led to a dramatic drop in Tₘ and T_{agg}.

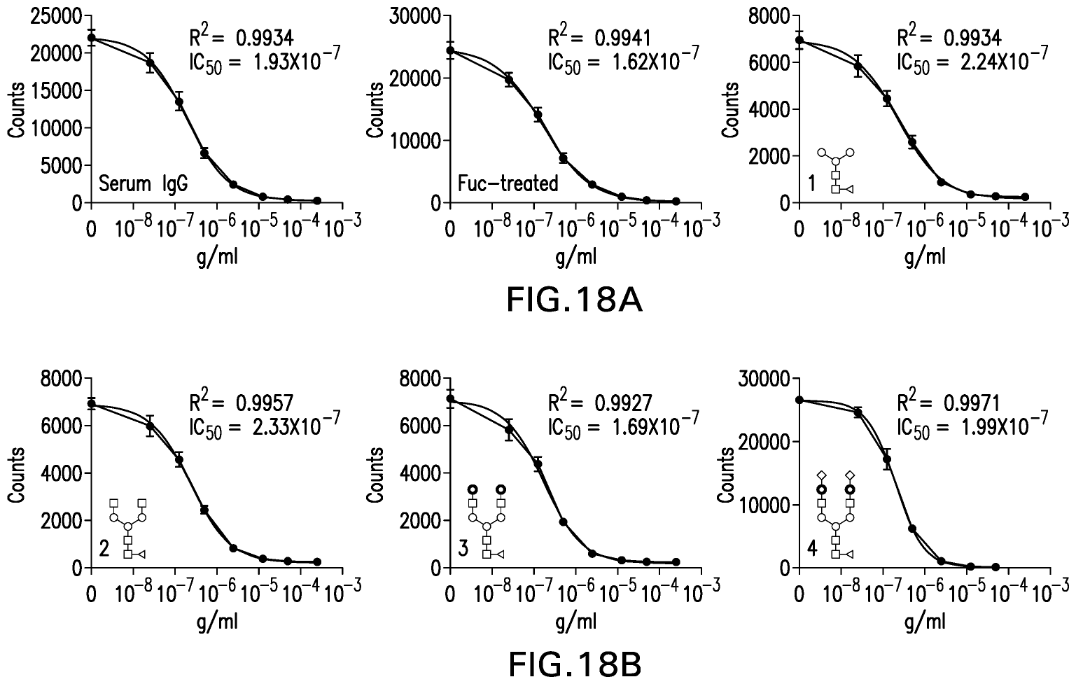

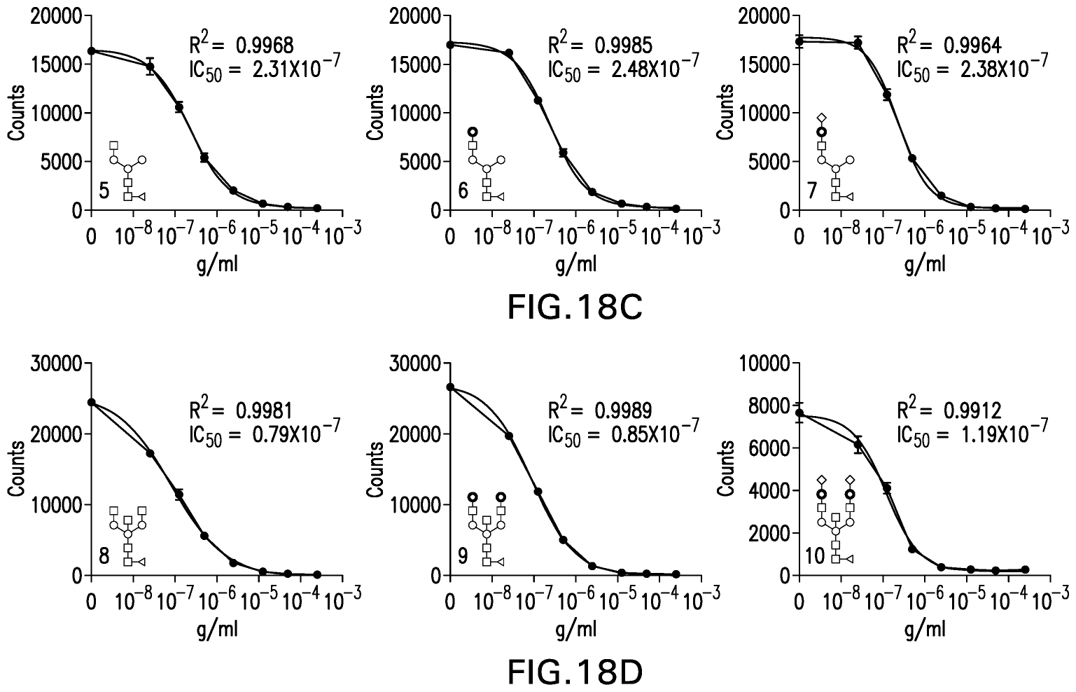

Next, we tested whether SPGR-engineered IgGs preserve the binding ability to FcRs. We performed a competition assay where the interaction between SPGR-engineered IgGs and Fc gamma receptor I (FcγR I) resulted in fluorescence signal reduction. A signal reduction of 60% was found in all the glycoengineered IgGs at the concentration of 0.1 μg/mL; while complete inhibitions were reached at about 10 μg/mL (FIG. 5D, FIG. 18). Furthermore, the bisecting glycoforms (van de Bovenkamp et al., Journal of Immunology 196, 1435-1441, doi: 10.4049/jimmunol. 1502136 (2016); Munkley, Oncol. Lett. 17, 2569-2575, doi: 10.3892/ol.2019.9885 (2019); Pinho & Reis, Nat. Rev.Cancer 15, 540-555, doi: 10.1038/nrc3982 (2015)) showed decreased EC₅₀ values in this assay, indicating an enhanced binding affinity to FcγR (Table 5). This result agreed with previous observations that mouse IgG1 bearing a higher level of bisecting glycoforms has increased ADCC activity (Davies et al., Biotechnology and Bioengineering 74, 288-294, doi: 10.1002/bit. 1119 (2001)). By contrast, IgGs bearing hybrid glycoforms (Reily et al., Nature Reviews Nephrology 15, 346-366, doi: 10.1038/s41581-019-0129-4 (2019); Maverakis et al., J. Autoimmun. 57, 1-13, doi: 10.1016/j.jaut.2014.12.002 (2015); Reiding et al., Front Med (Lausanne) 4, doi: 10.3389/fmed.2017.00241 (2017)) showed a slightly decreased binding affinity to FcγR I. Together, our characterization analyses suggested that: 1) SPGR-engineered IgGs have preserved integrity and functions, and 2) the difference in FcR binding affinity between hybrid, bi-antennary, and bisecting glycoforms could present a useful handle to control IgGs' immunogenicity.

Beyond the presented work, there remains an opportunity to explore alternative immobilization strategies in future experiments. IgG immobilization using protein A, a 47 kD protein, likely limited the enzyme efficiency by creating a strong steric hindrance in some cases (Apweiler et al., Biochim Biophys Acta 1473, 4-8, doi: 10.1016/s0304-4165 (99) 00165-8 (1999)). Methods using oligopeptides, such as a HIS-tag, presumably have a lower steric effect and may enable higher enzyme activities. Since immobilization has been commonly employed for protein purification in pharmaceutical manufacturing processes, SPGR can conceivably be inserted into modern protein production processes as a “glycan modification module” to provide pure, glycoengineered proteins.

### Example 3

### Comparison Between Substrate Immobilization and Enzyme Immobilization in SPGR

An alternative approach to conduct SPGR is immobilizing the glycosylation enzymes on solid supports instead of the IgG substrates (FIG. 19) (Li et al., Carbohydr. Res. 458-459, 77-84, doi: 10.1016/j.carres.2018.02.007 (2018)). In this way, glycosylation enzymes can be easily pulled out from the reaction pools for recovery. This could improve the efficiency of large-scale productions where the enzymes are re-charged in multiple batches of reactions. Furthermore, because substrates present the majority of the substance in the reactions, immobilizing the enzymes, instead of the substrate, could reduce the cost of immobilization in the production pipelines. However, there are also restrictions in this manner. For example, the selection of enzymes for the remodeling processes is much more limited in this case because the buffer of the IgG solution cannot be swapped easily. In a remodeling process involving both glycosidases and glycosyltransferases-which prefer very different conditions (pH, cations)—the use of a generic working buffer would significantly compromise the enzyme activities. In addition, the preparation of resin-immobilized glycosylation enzymes could be difficult.

Whether immobilization affects their activity and substrate selectivity, as well as the immobilization protocol itself, remains to be investigated.

### Endoglycosidases for Glycan Remodeling

IgG glycan trimming can also be implemented through the GlcNAc residues in the chitobiose core. Endoglycosidases specifically cleave the β1-4 linkage between the GlcNAc residues in the chitobiose core (Freeze et al., Curr. Protoc. Mol. Biol. Chapter 17, 10.1002/0471142727.mb0471141713as0471142789-0471142717.0471142713A, doi: 10.1002/0471142727.mb1713as89 (2010); Collin et al., EMBO J. 20, 3046-3055, doi: 10.1093/emboj/20.12.3046 (2001); Fan et al., Glycoconj J 13, 643-652, doi: 10.1007/bf00731453 (1996); Yamamoto et al., Biochem. Biophys. Res. Commun. 203, 244-252, doi: 10.1006/bbrc. 1994.2174 (1994)). They have been employed for chemoenzymatic glycan modification where the native glycans of targeted glycoprotein are first removed by endoglycosidases, followed by installing synthetic glycans back to the proteins using mutated endoglycosidases (glycosynthases) (Huang et al., J. Am. Chem. Soc. 134, 12308-12318, doi: 10.1021/ja3051266 (2012)). Since re-building the chitobiose cores remains challenging, largely due to the lack of available mannosyl-transferases, we analyzed the activity of endoglycosidases on intact IgG but did not apply them in SPGR applications. Of the six tested enzymes, the candidate from Streptococcus pyogenes (known as Endo S) exhibited the highest conversion ratio on intact IgG (CR50=0.7%, FIG. 20A-20D). It has been known that endoglycosidases have different preferences for substrate structures. In agreement with reported studies, Endo S effectively liberates N-glycans from human IgG in SPGR.8 On the other hand, the ones with higher specificity to high-mannose glycans, such as endoglycosidase D, did not show detectable IgG glycan conversion in our screening (Table 1) (Mizuochi et al., J. Biochem. 95, 1209-1213, doi: 10.1093/oxfordjournals.jbchem.a134711 (1984)). A complete Endo S reaction led to the removal of glycan majority, giving a clean chromatogram as shown in FIG. 20A-20D.

### Candidatus Omnitrophica Fucosidase has a Wide Spectrum on Substrate Selectivity

With the use of high enzyme concentration and long incubation time, we found that Fuc from C. omnitrophica functioned on all the IgG glycoforms. (FIG. 10C). While we also observed decreased activity as the structural complexity of the glycans increased (FIG. 10D), the broad spectrum of substrate selectivity makes this enzyme an attractive tool for glycan remodeling on intact IgG.

### Steric Hindrance Created by IgG Immobilization Reduces Glycosylation Enzyme Activities

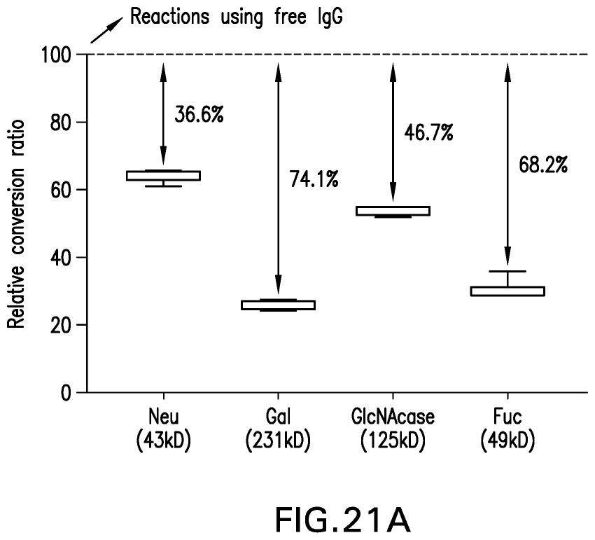

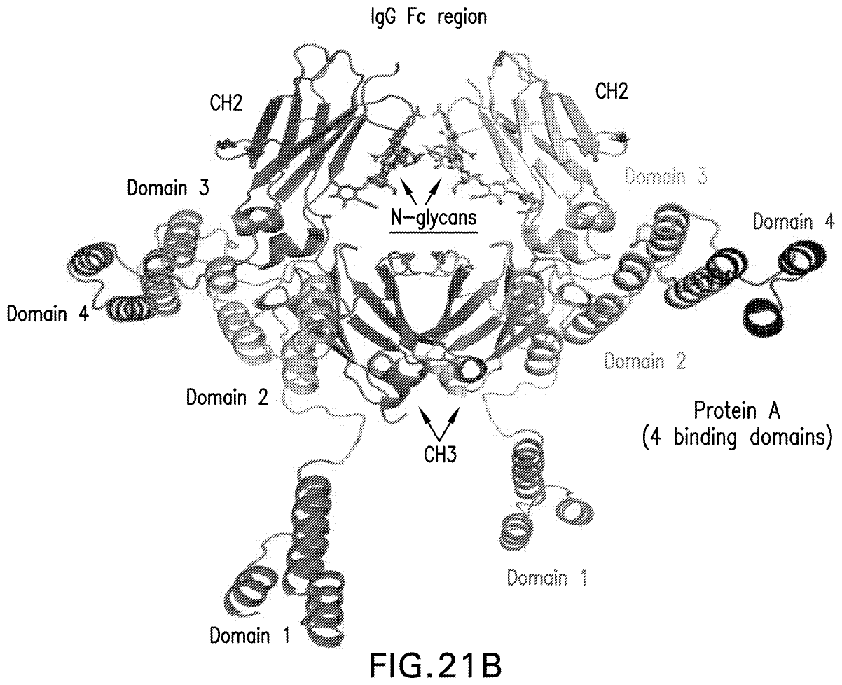

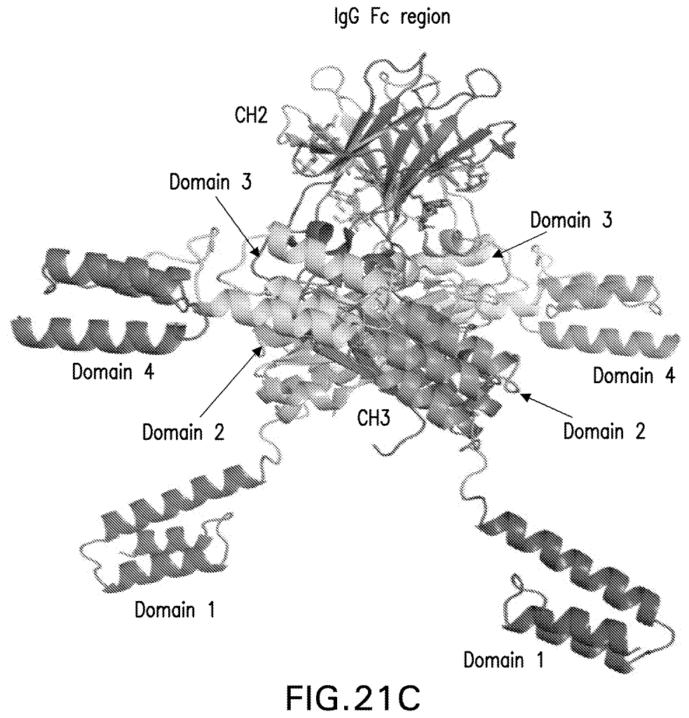

IgG immobilization enables efficient washing and reaction-swapping processes in SPGR. However, we observed reduced enzyme activities on immobilized IgG compared to non-immobilized, free IgG (FIG. 21A). Such a “trade-off” partially results from the relatively limited surface area in heterogeneous reactions but could mainly be attributed to the increased steric hindrance created by IgG binding to protein A. It's reported that protein A binds to the IgG Fc at the interface between the CH2 and CH3 domains (Deisenhofer, Biochemistry 20, 2361-2370, doi: 10.1021/bi00512a001 (1981)). This binding region is not only closed to but also interacting with the Asn297 glycans (Kiyoshi et al., International Immunology 29, 311-317, doi: 10.1093/intimm/dxx038 (2017)). Computational modeling of interactions between the full-length protein A (with four domains) and the Fc region indicates steric hindrance, specifically at the CH2 region (FIG. 21A-21C). This could lead to reduced accessibility of the glycans by glycosylation enzymes. This hypothesis of spatial hindrance and reduced accessibility is supported by the size effect of glycosylation enzymes whose reduced activity correlates with their molecular weight. For example, the largest enzyme in our toolset, Gal from S. pneumoniae (231 kD), showed an activity reduction of 74% when functions on immobilized IgG. A significant activity reduction was also found in Fuc despite its smaller size, an enzyme that enables chemistry at the very bottom of the glycan structure. In addition to steric hindrance, multiple IgG Fc regions could be interacting with the same protein A molecule, thus leading to a crowing effect and reduced enzymatic activity.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1. A method for remodeling the N-glycans of a composition of N-glycosylated glycoproteins, the steps comprising:
(a) providing an aqueous composition of N-glycosylated glycoproteins;
(b) immobilizing the N-glycosylated glycoproteins on a solid support;
(c) reacting the immobilized N-glycosylated glycoproteins with an exoglycosidase or sequentially with two or more exoglycosidases to produce first immobilized N-glycosylated glycoproteins wherein the remodeled N-glycans thereof comprise two terminal mannose residues at the non-reducing end of the N-glycan, one terminal mannose linked to a central mannose residue in an α1-3 linkage and the other terminal mannose linked to the same central mannose in an α1-6 linkage, and the central mannose is linked to a chitobiose core in a β1-4 linkage wherein the GlcNAc residue at the reducing end of the chitobiose core is linked to an asparagine residue of an N-glycosylation site in the N-glycosylated glycoprotein;
(d) reacting the first immobilized N-glycosylated glycoproteins with a GnTI in the presence of UDP-GlcNAc to produce second immobilized N-glycosylated glycoproteins wherein the remodeled N-glycans thereof further comprise a GlcNAc residue linked in a β1-2 linkage to the non-reducing end of the α1-3-linked mannose residue; and
(e) eluting the second immobilized N-glycosylated glycoproteins comprising the remodeled N-glycans from the solid support to provide a composition of the N-glycosylated glycoproteins comprising remodeled N-glycans.

2. The method of claim 1, wherein the solid support comprises irreversibly affixed thereon a multiplicity of capture moieties that specifically bind the N-glycosylated glycoprotein.

3. The method of claim 1, wherein the N-glycosylated glycoprotein comprises an antibody or antigen binding fragment.

4. The method of claim 2, wherein the multiplicity of capture moieties comprises protein A and the N-glycosylated glycoprotein comprises an antibody.

5. The method of claim 1, wherein the one or more glycosylation enzymes are selected from the group consisting of exoglycosidases, endoglycosidases, and glycosyltransferases.

6. The method of claim 5, wherein the exoglycosidases are selected from the group consisting of neuraminidases, galactosidases, fucosidases, and N-acetyl-glucosaminidases; and, the glycosyltransferases are selected from the group consisting of N-acetylglucosaminidyltransferases (GnTs), galactosyltransferases (GalTs), fucosyltransferases (FuTs), and sialyltransferases (SiaTs).

7. The method of claim 6, wherein the SiaTs are selected from the group consisting of α2-6 SiaT and α2-3 SiaT; the GalTs comprise β1-4GalT; the FuTs comprise α1-6 FuT; and, the GnTs are selected from the group consisting of GnTI, GnTII, GnTIII, GnTIV, and GnTV.

8-9. (canceled)

10. The method of claim 1, wherein the second immobilized N-glycosylated glycoproteins are reacted with a GalT in the presence of UDP-galactose to produce third immobilized N-glycosylated glycoproteins wherein the remodeled N-glycans thereof further comprise a galactose residue linked in a β1-4 linkage to the non-reducing end of the GlcNAc residue linked to the non-reducing end of the α1-3-linked mannose residue.

11. The method of claim 10, wherein the third immobilized N-glycosylated glycoproteins are reacted with a SiaT in the presence of CMP-sialic acid to produce fourth immobilized N-glycosylated glycoproteins wherein the remodeled N-glycans thereof further comprise a sialic acid residue linked to the non-reducing end of the galactose residue linked to the non-reducing end of the GlcNAc residue linked in an α2-3 or α2-6 linkage to the non-reducing end of the α1-3-linked mannose residue.

12. The method of claim 11, wherein the second immobilized N-glycosylated glycoproteins are reacted with a GnTII in the presence of UDP-GlcNAc to produce third immobilized N-glycosylated glycoproteins wherein the remodeled N-glycans thereof further comprise a GlcNAc residue linked in a β1-2 linkage to the non-reducing end of the α1-6-linked mannose residue.

13. The method of claim 12, wherein the third immobilized N-glycosylated glycoproteins are reacted with a GalT in the presence of UDP-galactose to produce fourth immobilized N-glycosylated glycoproteins wherein the remodeled N-glycans thereof further comprise:
(a) a galactose residue linked in a β1-4 linkage to the non-reducing end of the GlcNAc residue linked in a β1-2 linkage to the non-reducing end of the α1-3-linked mannose residue,
(b) a galactose residue linked in a β1-4 linkage to the non-reducing end of the GlcNAc residue linked in a β1-2 linkage to the non-reducing end of the α1-6-linked mannose residue, or
(c) a galactose residue linked in a β1-4 linkage to the non-reducing end of the GlcNAc residue linked in a β1-2 linkage to the non-reducing end of the α1-3-linked mannose residue and a galactose residue linked in a β1-4 linkage to the non-reducing end of the GlcNAc residue linked in a β1-2 linkage to the non-reducing end of the α1-6-linked mannose residue.

14. The method of claim 13, wherein the fourth immobilized N-glycosylated glycoproteins are reacted with a SiaT in the presence of CMP-sialic acid to produce fifth immobilized N-glycosylated glycoproteins wherein the remodeled N-glycans thereof further comprise:
(a) a sialic acid residue linked in an α2-3 or α2-6 linkage to the non-reducing end of the galactose residue linked in a β1-4 linkage to the non-reducing end of the GlcNAc residue linked in a β1-2 linkage to the non-reducing end of the α1-3-linked mannose residue,
(b) a sialic acid residue linked in an α2-3 or α2-6 linkage to the non-reducing end of the galactose residue linked in a β1-4 linkage to the non-reducing end of the GlcNAc residue linked in a β1-2 linkage to the non-reducing end of the α1-6-linked mannose residue, or
(c) a sialic acid residue linked in an α2-3 or α2-6 linkage to the non-reducing end of the galactose residue linked in a β1-4 linkage to the non-reducing end of the GlcNAc residue linked in a β1-2 linkage to the non-reducing end of the α1-3-linked mannose residue and a sialic acid residue linked in an α2-3 or α2-6 linkage to the non-reducing end of the galactose residue linked in a β1-4 linkage to the non-reducing end of the GlcNAc residue linked in a β1-2 linkage to the non-reducing end of the α1-6-linked mannose residue.

15. The method of claim 12, wherein the third immobilized N-glycosylated glycoproteins are reacted with a GnTIII in the presence of UDP-GlcNAc to produce fourth immobilized N-glycosylated glycoproteins wherein the remodeled N-glycans thereof further comprise a GlcNAc residue linked in a β1-4 linkage to the non-reducing end of the central mannose residue.

16. The method of claim 15, wherein the fourth immobilized N-glycosylated glycoproteins are reacted with a GalT in the presence of UDP-galactose to produce fifth immobilized N-glycosylated glycoproteins wherein the remodeled N-glycans thereof further comprise:
(a) a galactose residue linked in a β1-4 linkage to the non-reducing end of the GlcNAc residue linked in a β1-2 linkage to the non-reducing end of the α1-3-linked mannose residue,
(b) a galactose residue linked in a β1-4 linkage to the non-reducing end of the GlcNAc residue linked in a β1-2 linkage to the non-reducing end of the α1-6-linked mannose residue, or
(c) a galactose residue linked in a β1-4 linkage to the non-reducing end of the GlcNAc residue linked to the non-reducing end of the α1-3-linked mannose residue and a galactose residue linked in a β1-4 linkage to the non-reducing end of the GlcNAc residue linked in a β1-2 linkage to the non-reducing end of the α1-6-linked mannose residue.

17. The method of claim 16, wherein the fifth immobilized N-glycosylated glycoproteins are reacted with a SiaT in the presence of CMP-sialic acid to produce sixth immobilized N-glycosylated glycoproteins wherein the remodeled N-glycans thereof further comprise:
(a) a sialic acid residue linked in an α2-3 or α2-6 linkage to the non-reducing end of the galactose residue linked in a β1-4 linkage to the non-reducing end of the GlcNAc residue linked in a β1-2 linkage to the non-reducing end of the α1-3-linked mannose residue,
(b) a sialic acid residue linked in an α2-3 or α2-6 linkage to the non-reducing end of the galactose residue linked in a β1-4 linkage to the non-reducing end of the GlcNAc residue linked in a β1-2 linkage to the non-reducing end of the α1-6-linked mannose residue, or
(c) a sialic acid residue linked in an α2-3 or α2-6 linkage to the non-reducing end of the galactose residue linked in a β1-2 linkage to the non-reducing end of the GlcNAc residue linked in a 1-2 linkage to the non-reducing end of the α1-3-linked mannose residue and a sialic acid residue linked in an α2-3 or α2-6 linkage to the non-reducing end of the galactose residue linked in a β1-2 linkage to the non-reducing end of the GlcNAc residue linked in a β1-2 linkage to the non-reducing end of the α1-6-linked mannose residue.

18. The method of claim 12, wherein the third immobilized N-glycosylated glycoproteins are reacted with a GnTIV in the presence of UDP-GlcNAc to produce fourth immobilized N-glycosylated glycoproteins wherein the remodeled N-glycans thereof further comprise a GlcNAc residue linked in a β1-4 linkage to the non-reducing end of the α1-3-linked mannose residue.

19. The method of claim 18, wherein the fourth immobilized N-glycosylated glycoproteins are reacted with a GalT in the presence of UDP-galactose to produce fifth immobilized N-glycosylated glycoproteins wherein the remodeled N-glycans thereof further comprise:
(a) a galactose residue linked in a β1-4 linkage to the non-reducing end of the GlcNAc residue linked in a β1-2 linkage to the non-reducing end of the α1-3-linked mannose residue;
(b) a galactose residue linked in a β1-4 linkage to the non-reducing end of the GlcNAc residue linked in a β1-2 linkage to the non-reducing end of the α1-6-linked mannose residue;
(c) a galactose residue linked in a β1-4 linkage to the non-reducing end of the GlcNAc residue linked in a β1-4 linkage to the non-reducing end of the α1-3-linked mannose residue; or
(d) a combination of two or more selected from the group consisting of (a), (b), and (c).

20. The method of claim 19, wherein the fifth immobilized N-glycosylated glycoproteins are reacted with a SiaT in the presence of CMP-sialic acid to produce sixth immobilized N-glycosylated glycoproteins wherein the remodeled N-glycans thereof further comprise:
(a) a sialic acid residue linked in an α2-3 or α2-6 linkage to the non-reducing end of the galactose residue linked in a β1-4 linkage to the non-reducing end of the GlcNAc residue linked in a β1-2 linkage to the non-reducing end of the α1-3-linked mannose residue;
(b) a sialic acid residue linked in an α2-3 or α2-6 linkage to the non-reducing end of the galactose residue linked in a β1-4 linkage to the non-reducing end of the GlcNAc residue linked in a β1-2 linkage to the non-reducing end of the α1-6-linked mannose residue;
(c) a sialic acid residue linked in an α2-3 or α2-6 linkage to the non-reducing end of the galactose residue linked in a β1-4 linkage to the non-reducing end of the GlcNAc residue linked in a β1-4 linkage to the non-reducing end of the α1-3-linked mannose residue; or
(d) a combination of two or more selected from the group consisting of (a), (b), and (c).

21. The method of claim 18, wherein the fourth immobilized N-glycosylated glycoproteins are reacted with a GnTV in the presence of UDP-GlcNAc to produce fifth immobilized N-glycosylated glycoproteins wherein the remodeled N-glycans thereof further comprise a GlcNAc residue linked in a β1-6 linkage to the non-reducing end of the α1-6-linked mannose residue.

22. The method of claim 21, wherein the fifth immobilized N-glycosylated glycoproteins are reacted with a GalT in the presence of UDP-galactose to produce sixth immobilized N-glycosylated glycoproteins wherein the remodeled N-glycans thereof further comprise:
(a) a galactose residue linked in a β1-4 linkage to the non-reducing end of the GlcNAc residue linked in a β1-2 linkage to the non-reducing end of the α1-3-linked mannose residue;
(b) a galactose residue linked in a β1-4 linkage to the non-reducing end of the GlcNAc residue linked in a β1-2 linkage to the non-reducing end of the α1-6-linked mannose residue;
(c) a galactose residue linked in a β1-4 linkage to the non-reducing end of the GlcNAc residue linked in a β1-4 linkage to the non-reducing end of the α1-3-linked mannose residue;
(d) a galactose residue linked in a β1-4 linkage to the non-reducing end of the GlcNAc residue linked in a β1-6 linkage to the non-reducing end of the α1-6-linked mannose residue; or
(e) a combination of two or more selected from the group consisting of (a), (b), (c), and (d).

23. The method of claim 22, wherein the sixth immobilized N-glycosylated glycoproteins are reacted with a SiaT in the presence of CMP-sialic acid to produce seventh immobilized N-glycosylated glycoproteins wherein the remodeled N-glycans thereof further comprise:
(a) a sialic acid residue linked in an α2-3 or α2-6 linkage to the non-reducing end of the galactose residue linked in a β1-4 linkage to the non-reducing end of the GlcNAc residue linked in a β1-2 linkage to the non-reducing end of the α1-3-linked mannose residue;
(b) a sialic acid residue linked in an α2-3 or α2-6 linkage to the non-reducing end of the galactose residue linked in a β1-4 linkage to the non-reducing end of the GlcNAc residue linked in a β1-2 linkage to the non-reducing end of the α1-6-linked mannose residue;
(c) a sialic acid residue linked in an α2-3 or α2-6 linkage to the non-reducing end of the galactose residue linked in a β1-4 linkage to the non-reducing end of the GlcNAc residue linked in a β1-4 linkage to the non-reducing end of the α1-3-linked mannose residue;
(d) a sialic acid residue linked in an α2-3 or α2-6 linkage to the non-reducing end of the galactose residue linked in a β1-4 linkage to the non-reducing end of the GlcNAc residue linked in a β1-6 linkage to the non-reducing end of the α1-6-linked mannose residue; or
(e) a combination of two or more selected from the group consisting of (a), (b), (c), and (d).

24-82. (canceled)
